(19) United States
(12) Patent Application Publication
(10) Pub. No.: US 2026/0216477 A1
(43) Pub. Date: Jul. 30, 2026

(54) Radial Access Catheter

(71) Applicant: MicroVention, Inc., Aliso Viejo, CA (US)

(72) Inventors: Evelyn Vasquez, Aliso Viejo, CA (US); Kailey Lewis, Aliso Viejo, CA (US); Sonny Tigno, Lake Forest, CA (US); Ronak Dholakia, Aliso Viejo, CA (US); Roland Guyon, Santa Ana, CA (US); Joseph Gulachenski, Trabuco Canyon, CA (US); Imran Khalid, La Palma, CA (US)

(73) Assignee: MicroVention, Inc., Aliso Viejo, CA (US)

(21) Appl. No.: 19/144,830

(22) PCT Filed: Jan. 23, 2024
(86) PCT No.: PCT/US2024/012642

§ 371 Date: Jun. 30, 2025

Related U.S. Application Data
(63) Continuation-in-part of application No. PCT/US2023/081956, filed on Nov. 30, 2023.
(60) Provisional application No. 63/481,164, filed on Jan. 23, 2023.
(60) Provisional application No. 63/516,474, filed on Jul. 28, 2023.

(51) Int. Cl. A61M 25/00

(52) U.S. Cl. CPC A61M 25/0053; A61M 2210/12

(57) ABSTRACT

A device may include an elongated catheter body having a proximal portion and a distal portion, wherein the distal portion forms a memorized shape when unconstrained; the memorized shape comprising a plurality of curves positioned to orient the distal portion within a right subclavian artery or brachiocephalic artery and also into a left common carotid artery.

### RELATED APPLICATIONS

This application claims benefit of and priority to U.S. Provisional Application Ser. No. 63/481,164 filed Jan. 23, 2023 entitled Radial Access Catheter, PCT Application No. PCT/US2023/081956 filed Nov. 30, 2023 entitled Inner Support Catheter, and U.S. Provisional Application Ser. No. 63/516,474 filed Jul. 28, 2023, entitled Inner Support Catheter, all of which are hereby incorporated herein by reference in their entireties.

### BACKGROUND

Catheter access to regions of a patient's body, such as the heart or brain, is most commonly achieved by entering the patient's vasculature at the femoral artery. This approach is desirable, in part, because of relatively large size of the femoral artery and the relatively straight path it provides to the iliac artery and then on to the aorta, from which a variety of different locations may be accessed with a catheter.

However, in some circumstances, radial access (i.e., access via the radial artery near a patient's wrist) may be necessary or desirable. Some example advantages of radial access relative to femoral access may include (1) reduced duration of post procedure bed rest and length of stay, enhancing patient comfort; (2) lower incidence of access site complications, including bleeding, pseudoaneurysm, and arteriovenous fistulas; and (3) potential reductions in overall costs. Further, some patients may have relatively tortuous vessels that make femoral access challenging.

Navigating catheters through the aortic arch to access the cerebral vasculature can be challenging for several reasons. First, the aortic arch has a complex anatomy that varies among individuals. The shape, size, and angle of the aortic arch and its branches can affect the ease and safety of catheterization. Second, the aortic arch is subject to hemodynamic forces that can influence the movement and stability of catheters. The blood flow and pressure in the aortic arch can cause catheters to buckle, kink, or dislodge during navigation. Third, the aortic arch is a potential source of emboli that can cause stroke or other complications. Catheter manipulation in the aortic arch can dislodge plaque or thrombus from the aortic wall or its branches and cause embolic events.

### SUMMARY

In some aspects, the techniques described herein relate to a select catheter, including: an elongated catheter body including a distal region with a constrained linear shape and an unconstrained shape; wherein the distal region includes a distal first section and a proximal second section that forms a major curve with the proximal second section in the unconstrained shape, wherein the distal first section is distally located relative to the proximal second section in the constrained linear shape; and, where in the unconstrained shape: the proximal second section lies substantially in a first reference plane; and, the distal first section is positioned at least partially outside of the first reference plane.

In some aspects, the techniques described herein relate to a select catheter, wherein a distal tip of the distal first section is positioned proximally of the major curve.

In some aspects, the techniques described herein relate to a select catheter, wherein the distal first section is positioned at an angle within an inclusive range of 5 and 25 degrees relative to the first reference plane.

In some aspects, the techniques described herein relate to a select catheter, wherein the distal first section is positioned at an angle of about 11, 11.01, 11.02, 11.03, 11.04, 11.05, 11.06, 11.07, 11.08, 11.09, 11.1, 11.11, 11.12, 11.13, 11.14, 11.15, 11.16, 11.17, 11.18, 11.19, 11.2, 11.21, 11.22, 11.23, 11.24, 11.25, 11.26, 11.27, 11.28, 11.29, 11.3, 11.31, 11.32, 11.33, 11.34, 11.35, 11.36, 11.37, 11.38, 11.39, 11.4, 11.41, 11.42, 11.43, 11.44, 11.45, 11.46, 11.47, 11.48, 11.49, 11.5, 11.51, 11.52, 11.53, 11.54, 11.55, 11.56, 11.57, 11.58, 11.59, 11.6, 11.61, 11.62, 11.63, 11.64, 11.65, 11.66, 11.67, 11.68, 11.69, 11.7, 11.71, 11.72, 11.73, 11.74, 11.75, 11.76, 11.77, 11.78, 11.79, 11.8, 11.81, 11.82, 11.83, 11.84, 11.85, 11.86, 11.87, 11.88, 11.89, 11.9, 11.91, 11.92, 11.93, 11.94, 11.95, 11.96, 11.97, 11.98, 11.99, or 12 degrees relative to the first reference plane.

In some aspects, the techniques described herein relate to a select catheter, wherein a distal tip of the distal first section is positioned at about inclusive range of about 0.4 cm to 2.0 cm away from a face of the first reference plane.

In some aspects, the techniques described herein relate to a select catheter, wherein the distal tip of the distal first section is positioned at a length from the proximal second section that is about parallel to the first reference plane within an inclusive range of about 1 cm to about 2 cm away from the proximal second section.

In some aspects, the techniques described herein relate to a select catheter, wherein the distal first section further includes a first minor curve.

In some aspects, the techniques described herein relate to a select catheter, wherein the first minor curve curves in a direction generally away from the proximal second section.

In some aspects, the techniques described herein relate to a select catheter, wherein the first minor curve has a curvature less than a curvature of the major curve.

In some aspects, the techniques described herein relate to a select catheter, wherein the first minor curve has a curvature within an inclusive range of about 90 to about 190 degrees.

In some aspects, the techniques described herein relate to a select catheter, wherein the distal first section is within an inclusive range of about 3 cm to 8 cm in length.

In some aspects, the techniques described herein relate to a select catheter, wherein the distal first section is positioned at an angle within an inclusive range of 5 and 25 degrees relative to the first reference plane; wherein a distal tip of the distal first section is positioned at about inclusive range of about 0.4 cm to 2.0 cm away from a face of the first reference plane; wherein the distal tip of the distal first section is positioned at a length from the proximal second section that is about parallel to the first reference plane within an inclusive range of about 1 cm to about 2 cm away from the proximal second section; wherein the first minor curve has a curvature within an inclusive range of about 90 to about 190 degrees; wherein the distal first section is within an inclusive range of about 3 cm to 8 cm in length.

In some aspects, the techniques described herein relate to a select catheter, wherein the select catheter has a stiffness at about 110 mm from a distal tip of the elongated catheter body of about 136.76 gf, about 80 mm from the distal tip of about 110.34 gf, at about 20 mm from the distal tip of about 22.58 gf, at about 10 mm from the distal tip of about 13.5 gf, and at about 5 mm from the distal tip of about 8 gf.

In some aspects, the techniques described herein relate to a select catheter, wherein the select catheter has a stiffness at about 10 mm within a range of about 6.75 gf and 20.25 gf.

In some aspects, the techniques described herein relate to a select catheter, wherein the select catheter has a stiffness at about 20 mm within a range of about 11.29 gf and 33.86 gf.

In some aspects, the techniques described herein relate to a select catheter, wherein the distal first section is positioned at an angle of about 10.0, 10.05, 10.1, 10.15, 10.2, 10.25, 10.3, 10.35, 10.4, 10.45, 10.5, 10.55, 10.6, 10.65, 10.7, 10.75, 10.8, 10.85, 10.9, 10.95, or 11.0 degrees relative to the first reference plane.

In some aspects, the techniques described herein relate to a select catheter, wherein a distal tip of the distal first section is positioned within inclusive range of about 0.1 cm to about 1.0 cm away from a face of the first reference plane.

In some aspects, the techniques described herein relate to a select catheter, wherein the distal tip of the distal first section is positioned at a length from the proximal second section that is about parallel to the first reference plane within an inclusive range of about .2 cm to about .5 cm away from the proximal second section.

In some aspects, the techniques described herein relate to a select catheter, wherein the distal first section is within an inclusive range of about 2 cm to 5 cm in length.

In some aspects, the techniques described herein relate to a select catheter, wherein the distal first section is positioned at an angle within an inclusive range of 5 and 25 degrees relative to the first reference plane; wherein a distal tip of the distal first section is positioned at about inclusive range of about 0.1 cm to 1.0 cm away from a face of the first reference plane; wherein the distal tip of the distal first section is positioned at a length from the proximal second section that is about parallel to the first reference plane within an inclusive range of about .2 cm to about .5 cm away from the proximal second section; wherein the first minor curve has a curvature within an inclusive range of about 90 to about 190 degrees; wherein the distal first section is within an inclusive range of about 2 cm to 5 cm in length.

In some aspects, the techniques described herein relate to a select catheter, wherein the select catheter has a stiffness at about 110 mm from a distal tip of the elongated catheter body of about 154.85 gf, about 80 mm from the distal tip of about 128.99 gf, at about 20 mm from the distal tip of about 38.96 gf, at about 10 mm from the distal tip of about 12.08 gf, and at about 5 mm from the distal tip of about 6.40 gf.

In some aspects, the techniques described herein relate to a select catheter, wherein the select catheter has a stiffness at about 10 mm within a range of about 6.04 gf and 18.11 gf.

In some aspects, the techniques described herein relate to a select catheter, wherein the select catheter has a stiffness at about 20 mm within a range of about 19.48 gf and 58.44 gf.

In some aspects, the techniques described herein relate to a select catheter, including: an elongated catheter body including a distal region with an unconstrained shape; wherein the distal region includes a first distal section that forms a major curve with a second distal section in the unconstrained shape; and, wherein the unconstrained shape the first distal section is positioned at a non-parallel angle relative to the second distal section and wherein a free distal tip of the distal region is positioned in a generally proximal orientation.

In some aspects, the techniques described herein relate to a select catheter, including: an elongated catheter body including a distal region with an unconstrained shape means for accessing a right common carotid artery or left common carotid artery via radial access; wherein the distal region includes a first distal section that forms a major curve with a second distal section.

In some aspects, the techniques described herein relate to a select catheter, including: an elongated catheter body; wherein the elongated catheter body has a stiffness at about 110 mm from a distal tip within an inclusive range of about 68.38 gf and 205.14 gf; a stiffness at about 80 mm from a distal tip within an inclusive range of about 55.17 gf and 165.51 gf; a stiffness at about 20 mm within an inclusive range of about 11.29 gf and 33.86 gf; a stiffness at about 10 mm within an inclusive range of about 6.75 gf and 20.25 gf; and a stiffness at about 5 mm within an inclusive range of about 4 gf and 12 gf.

In some aspects, the techniques described herein relate to a select catheter, wherein the elongated catheter body has a stiffness at about 110 mm from the distal tip of about 136.76 gf, about 80 mm from the distal tip of about 110.34 gf, at about 20 mm from the distal tip of about 22.58 gf, at about 10 mm from the distal tip of about 13.5 gf, and at about 5 mm from the distal tip of about 8 gf.

In some aspects, the techniques described herein relate to a select catheter, including: an elongated catheter body; wherein the elongated catheter body has a stiffness at about 110 mm from a distal tip within an inclusive range of about 77.42 gf and 232.27 gf; a stiffness at about 80 mm from the distal tip of the select catheter within an inclusive range of about 64.50 gf and 193.49 gf; a stiffness at about 20 mm from the distal tip within an inclusive range of about 19.48 gf and 58.44 gf; a stiffness at about 10 mm from the distal tip within an inclusive range of about 6.04 gf and 18.11 gf; and a stiffness at about 5 mm from the distal tip within an inclusive range of about 3.20 gf and 9.60 gf.

In some aspects, the techniques described herein relate to a select catheter, wherein the elongated catheter body has a stiffness at about 110 mm from the distal tip of about 154.85 gf, about 80 mm from the distal tip of about 128.99 gf, about 20 mm from the distal tip of about 38.96 gf, about 10 mm from the distal tip of about 12.08 gf, and about 5 mm from the distal tip of about 6.40 gf.

In some aspects, the techniques described herein relate to a select catheter, including: an elongated catheter body; wherein the elongated catheter body has a stiffness of either of the following: wherein the elongated catheter body has a stiffness at about 110 mm from a distal tip of the elongated catheter body of about 136.76 gf, about 80 mm from the distal tip of about 110.34 gf, at about 20 mm from the distal tip of about 22.58 gf, at about 10 mm from the distal tip of about 13.5 gf, and at about 5 mm from the distal tip of about 8 gf; or, wherein the elongated catheter body has a stiffness at about 110 mm from the distal tip of about 154.85 gf, about 80 mm from the distal tip of about 128.99 gf, about 20 mm from the distal tip of about 38.96 gf, about 10 mm from the distal tip of about 12.08 gf, and about 5 mm from the distal tip of about 6.40 gf.

In some aspects, the techniques described herein relate to a catheter, including: an elongated catheter body having a proximal portion and a distal portion; wherein the distal portion forms a memorized shape when unconstrained; the memorized shape including a plurality of curves positioned to orient the distal portion within a right or left subclavian artery or brachiocephalic artery to access a left or right common carotid artery.

In some aspects, the techniques described herein relate to a catheter, wherein the memorized shape of the distal portion is a “U” shape further curved in a generally perpendicular direction relative to an apex of the “U” shape.

In some aspects, the techniques described herein relate to a catheter, wherein the memorized shape has dimensions within a following inclusive ranges: a length of about 3.0 cm to 8.5 cm, a width of about 1.5 cm to 3.0 cm, and a height of about 1.5 cm to 3.5 cm.

In some aspects, the techniques described herein relate to a catheter, wherein the distal portion further includes a proximal region, an intermediate region, and a distal region, and wherein a first curve of the plurality of curves is located between the proximal region and the intermediate region and has an angle of curvature within an inclusive range of about 35 degrees to about 75 degrees.

In some aspects, the techniques described herein relate to a catheter, wherein a second curve of the plurality of curves is located between the intermediate region and the distal region and has an angle of curvature within an inclusive range of about 160 degrees to about 200 degrees.

In some aspects, the techniques described herein relate to a catheter, wherein some of the plurality of curves are located within the distal region and have angles of curvature within an inclusive range of about 5 degrees to 30 degrees.

In some aspects, the techniques described herein relate to a method for accessing a left common carotid artery, including: advancing a distal portion of a select catheter from a right subclavian artery and into an aortic arch of a patient; allowing the distal portion of the select catheter to expand to a memorized shape in the aortic arch of the patient; and proximally retracting some of distal portion of the select catheter partially back into the right subclavian artery or a brachiocephalic artery such that a distal region of the distal portion moves into the left common carotid artery.

In some aspects, the techniques described herein relate to a method, wherein the memorized shape of the distal portion is a “U” shape further curved in a generally perpendicular direction relative to an apex of the “U” shape.

In some aspects, the techniques described herein relate to a catheter, including: an elongated catheter body having a proximal portion and a distal portion; wherein the distal portion forms a memorized shape when unconstrained; the memorized shape including a plurality of curves positioned to orient the distal portion within a right subclavian artery or brachiocephalic artery and also into a right common carotid artery.

In some aspects, the techniques described herein relate to a catheter, wherein the memorized shape of the distal portion is a “U” shape having a width within an inclusive range of about 0.7 cm to about 2.9 cm.

In some aspects, the techniques described herein relate to a catheter, wherein the distal portion includes a first curve with an angle of curvature within an inclusive range of about 70 degrees to 140 degrees, as well as a second curve, third curve, and a fourth curve that each have angles of curvature within an inclusive range of about 50-140 degrees.

In some aspects, the techniques described herein relate to a method for accessing a right common carotid artery, including: advancing a distal portion of a select catheter into a right subclavian artery or a brachiocephalic artery of a patient; further distally advancing the distal portion of the select catheter so that it curves to a memorized shape in the right subclavian artery or the brachiocephalic artery; and further distally advancing the curved memorized shape so that a portion of the distal portion enters the right common carotid artery.

In some aspects, the techniques described herein relate to a method, wherein the memorized shape of the distal portion is a “U” shape having a width within an inclusive range of about 0.7 cm to about 2.9 cm.

In some aspects, the techniques described herein relate to a catheter, including: an elongated catheter body having a proximal portion and a distal portion; wherein the distal portion forms a memorized shape when unconstrained; the memorized shape including a primary curve having an angle of curvature within an inclusive range of about 335 to about 350 degrees positioned to orient the distal portion within a right subclavian artery or brachiocephalic artery and also into a right common carotid artery.

In some aspects, the techniques described herein relate to a catheter, wherein the primary curve has a width within an inclusive range of about 0.5 cm to about 2.5 cm.

In some aspects, the techniques described herein relate to a catheter, including: an elongated catheter body having a proximal portion and a distal portion; wherein the distal portion has a constrained configuration and an unconstrained configuration different from the constrained configuration, and wherein in the unconstrained configuration, the catheter shape includes a plurality of curves that deflect at least in part relative to an axial “X” axis through a central lumen of the proximal portion of the catheter, a vertical “Y” axis perpendicular to the “X” axis, and a lateral “Z” axis perpendicular to both the “X” and “Y” axes.

In some aspects, the techniques described herein relate to a catheter, wherein the plurality of curves are positioned to orient the distal portion of the catheter for access to a specific target vessel.

In some aspects, the techniques described herein relate to a catheter, wherein the specific target vessel is one of a right or left common carotid artery.

In some aspects, the techniques described herein relate to a catheter, wherein the distal portion of the catheter in the unconstrained configuration has a memorized shape, and wherein at least a portion of the memorized shape of the distal portion includes a “U” shape in the unconstrained configuration, wherein the “U” shape has a proximal section, a medial section, and a distal section, wherein the proximal section of the “U” shape extends at least in part along the “X” axis away from the proximal portion of the catheter, and wherein the distal section of the “U” shape extends at least in part along the “X” axis towards the proximal portion of the catheter.

In some aspects, the techniques described herein relate to a catheter, including: an elongated catheter means for accessing a common carotid artery, wherein the catheter means has a body with a proximal portion and a distal portion; wherein the distal portion has a constrained configuration and an unconstrained configuration different from the constrained configuration, and wherein the elongated catheter means forms a memorized shape when unconstrained, the memorized shape including a plurality of curves to orient the distal portion within a right or left subclavian artery or brachiocephalic artery to access a left common carotid artery.

### BRIEF DESCRIPTION OF THE DRAWINGS

The following figures are included to illustrate certain example aspects of the present disclosure and should not be viewed as exclusive or limiting. The subject matter disclosed is capable of considerable modifications, alterations, combinations, and equivalents in form and function, as will occur to one having ordinary skill in the art and having the benefit of this disclosure. The present disclosure references the drawings as follows:

FIG. 1 illustrates a general overview of some arteries of a typical human 10.

FIG. 2A illustrates a side view of an example aortic arch 24 of a human.

FIG. 2B illustrates side views of several different examples of aortic arch 24 anatomies of a human.

FIG. 3 illustrates a side view of a distal portion of a select catheter 100 for access to the left common carotid artery 32 from the right subclavian artery 16 (i.e., a right radial access approach) according to one example.

FIG. 4 illustrates a side view of a distal portion of a select catheter 100 for access to the left common carotid artery 32 from the right subclavian artery 16 (i.e., a right radial access approach) according to one example.

FIG. 5 illustrates a side view of a distal portion of a select catheter 100 for access to the left common carotid artery 32 from the right subclavian artery 16 (i.e., a right radial access approach) according to one example.

FIG. 6 illustrates a side view of a distal portion of a select catheter 100 for access to the left common carotid artery 32 from the right subclavian artery 16 (i.e., a right radial access approach) according to one example.

FIG. 7 illustrates a side view of a distal portion of a select catheter 100 for access to the left common carotid artery 32 from the right subclavian artery 16 (i.e., a right radial access approach) according to one example.

FIG. 8 illustrates a side view of a distal portion of a select catheter 100 for access to the left common carotid artery 32 from the right subclavian artery 16 (i.e., a right radial access approach) according to one example.

FIG. 9 illustrates a side view of a distal portion of a select catheter 100 for access to the left common carotid artery 32 from the right subclavian artery 16 (i.e., a right radial access approach) according to one example.

FIG. 10 illustrates a side view of a distal portion of a select catheter 100 for access to the left common carotid artery 32 from the right subclavian artery 16 (i.e., a right radial access approach) according to one example.

FIG. 11 illustrates a side view of a distal portion of a select catheter 100 for access to the left common carotid artery 32 from the right subclavian artery 16 (i.e., a right radial access approach) according to one example.

FIG. 12 illustrates a side view of a distal portion of a select catheter 100 for access to the left common carotid artery 32 from the right subclavian artery 16 (i.e., a right radial access approach) according to one example.

FIG. 13 illustrates a side view of a distal portion of a select catheter 100 for access to the left common carotid artery 32 from the right subclavian artery 16 (i.e., a right radial access approach) according to one example.

FIG. 14 illustrates a side view of a distal portion of a select catheter 100 for access to the left common carotid artery 32 from the right subclavian artery 16 (i.e., a right radial access approach) according to one example.

FIG. 15 illustrates an end view of a distal portion of a select catheter 100 for access to the left common carotid artery 32 from the right subclavian artery 16 (i.e., a right radial access approach) according to one example.

FIG. 16 illustrates an end view of a distal portion of a select catheter 100 for access to the left common carotid artery 32 from the right subclavian artery 16 (i.e., a right radial access approach) according to one example.

FIG. 17 illustrates a perspective view of a distal portion of a select catheter 100 for access to the left common carotid artery 32 from the right subclavian artery 16 (i.e., a right radial access approach) according to one example.

FIG. 18 illustrates a side view of an aortic arch 24 demonstrating a method of using the select catheter 100 according to one example.

FIG. 19 illustrates a side view of an aortic arch 24 demonstrating a method of using the select catheter 100 according to one example.

FIG. 20 illustrates a side view of an aortic arch 24 demonstrating a method of using the select catheter 100 according to one example.

FIG. 21 illustrates a chart of a stiffness profile of the select catheter 100 of FIGS. 3-17 according to one example.

FIG. 22 illustrates a side view of a select catheter 150 according to one example.

FIG. 23 illustrates a side view of a select catheter 150 according to one example.

FIG. 24 illustrates a side view of a select catheter 150 according to one example.

FIG. 25 illustrates a side view of a select catheter 150 according to one example.

FIG. 26 illustrates a side view of a select catheter 150 according to one example.

FIG. 27 illustrates an end view of a select catheter 150 according to one example.

FIG. 28 illustrates an end view of a select catheter 150 according to one example.

FIG. 29 illustrates a side view of a distal portion of a select catheter 160 for access to the right common carotid artery 30 from the right subclavian artery 16 (i.e., a right radial access approach) according to one example.

FIG. 30 illustrates a side view of a distal portion of a select catheter 160 for access to the right common carotid artery 30 from the right subclavian artery 16 (i.e., a right radial access approach) according to one example.

FIG. 31 illustrates a perspective view of a distal portion of a select catheter 160 for access to the right common carotid artery 30 from the right subclavian artery 16 (i.e., a right radial access approach) according to one example.

FIG. 32 illustrates a side view of a distal portion of a select catheter 160 for access to the right common carotid artery 30 from the right subclavian artery 16 (i.e., a right radial access approach) according to one example.

FIG. 33 illustrates a side view of an aortic arch 24 demonstrating a method of using the select catheter 160 according to one example.

FIG. 34 illustrates a side view of an aortic arch 24 demonstrating a method of using the select catheter 160 according to one example.

FIG. 35 illustrates a side view of an aortic arch 24 demonstrating a method of using the select catheter 160 according to one example.

FIG. 36 illustrates a chart of a stiffness profile of the select catheter 160 according to one example.

FIG. 37 illustrates a side view of a select catheter 190 according to one example.

FIG. 38 illustrates a side view of an aortic arch 24 demonstrating a method of using the select catheter 190 according to one example.

FIG. 39 illustrates a side view of the select catheter 200 in a first rotational position relative to a plane 200F according to one example.

FIG. 40 illustrates a side view of the select catheter 200 in a first rotational position relative to a plane 200F according to one example.

FIG. 41 illustrates an end view of the select catheter 200 from distal end perspective according to one example.

FIG. 42 illustrates an end view of the select catheter 200 from a proximal end perspective according to one example.

FIG. 43 illustrates a perspective view of the select catheter 200 according to one example.

FIG. 44 illustrates a side view of an aortic arch 24 demonstrating a method of using the select catheter 200 according to one example.

FIG. 45 illustrates a side view of an aortic arch 24 demonstrating a method of using the select catheter 200 according to one example.

FIG. 46 illustrates a chart of a stiffness profile of the select catheter 200 according to one example.

FIG. 47 illustrates a side view of the select catheter 300 in a first rotational position relative to a plane 300F according to one example.

FIG. 48 illustrates a side view of the select catheter 300 in a first rotational position relative to a plane 300F according to one example.

FIG. 49 illustrates an end view of the select catheter 300 in a first rotational position relative to a plane 300F according to one example.

FIG. 50 illustrates an end view of the select catheter 300 in a first rotational position relative to a plane 300F according to one example.

FIG. 51 illustrates a perspective view of the select catheter 300 from distal end perspective.

FIG. 52 illustrates a side view of an aortic arch 24 demonstrating a method of using the select catheter 300 according to one example.

FIG. 53 illustrates a side view of an aortic arch 24 demonstrating a method of using the select catheter 300 according to one example.

FIG. 54 illustrates a chart of a stiffness profile of the select catheter 300 according to one example.

FIG. 55 illustrates a side view of the select catheter 400 in a first rotational position relative to a plane 400F according to one example.

FIG. 56 illustrates a side view of the select catheter 400 in a first rotational position relative to a plane 400F according to one example.

FIG. 57 illustrates an end view of the select catheter 400 from distal end perspective according to one example.

FIG. 58 illustrates an end view of the select catheter 400 from a proximal end perspective according to one example.

FIG. 59 illustrates a perspective view of the select catheter 400 according to one example.

FIG. 60 illustrates a side view of an aortic arch 24 demonstrating a method of using the select catheter 300 according to one example.

FIG. 61 illustrates a side view of an aortic arch 24 demonstrating a method of using the select catheter 300 according to one example.

FIG. 62 illustrates a side view of the select catheter 400 according to one example.

FIG. 63 illustrates a side view of the select catheter 400 within a larger catheter 80 according to one example.

FIG. 64 illustrates a chart of a stiffness profile of the select catheter 200 according to one example.

### DETAILED DESCRIPTION

It will be appreciated by persons skilled in the art that the present disclosure is not limited to what has been particularly shown and described herein. A variety of modifications and variations are possible in view of the teachings herein without departing their scope, spirit, or intent.

While different examples may be described in this specification, it is specifically contemplated that any of the features from the different examples can be used and brought together in any combination. In other words, the features of different examples can be mixed and matched with each other. Hence, while every permutation of features from different examples may not be explicitly shown or described, it is the intention of this disclosure to cover any such combinations, especially as may be appreciated by one of skill in the art.

The terminology used in this disclosure should be interpreted in a permissive manner and is not intended to be limiting. In the drawings, like numbers refer to like elements. Unless otherwise noted, all of the accompanying drawings are not to scale. Unless otherwise noted, the term “about” is defined to mean plus-or-minus 5% of a stated value.

The terms distal or distally generally refer to a direction or area towards an end of a device within a patient (e.g., away from a physician/clinician), while the terms proximal or proximally refer to a direction or area toward an end of a device that remains outside of a patient (e.g., toward or closer to a physician/clinician or handle/hub of a device).

Any of the select catheters described in this specification may include an elongated catheter body have a proximal portion and a distal portion relative to where a physician may be located during a procedure. The select catheters may include a lumen extending between the proximal and distal ends of the select catheter which is at least large enough for use with a guidewire (e.g., 0.035″ to 0.038″ outer diameter guidewire).

While radial access to a patient's vasculature may have certain advantages, it can often be difficult for a physician to access some locations within a patient, particularly areas of a patient's brain due to relatively sharp and/or difficult orientations of some artery connections.

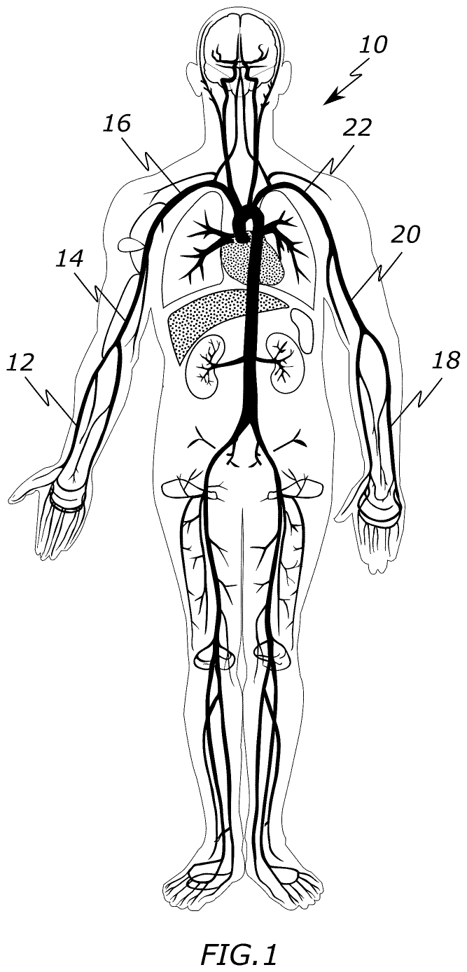

FIG. 1 illustrates a general overview of some arteries of a typical human 10. Radial access is typically first initiated by entering into a radial artery 12 within a patient's arm. Often, the right radial artery 12 is used as the initial point of entry for physicians as opposed to entry at the left radial artery 18. Physicians and neuro interventionalist physicians typically will orient patients in a headfirst supine position on the patient table with the patient imaging system screen on the left side of the patient. As a result, it is often easier to access and insert all devices from the right side. Additionally, depending on the desired location that is intended to be reached within the patient, the patient's arterial anatomy may provide further advantages to access via the right radial artery 12. However, access via the left radial artery 18 is possible and desirable in some circumstances.

In the case of right radial access, the right radial artery 12 is entered first (e.g., via a needle, introducer, and catheter), a guide catheter is advanced upwards into the right brachial artery 14, and then further into the right subclavian artery 16. Similarly, in the case of left radial access, the left radial artery 18 is entered first, the catheter is advanced upwards into the left brachial artery 20, and then further into the left subclavian artery 22.

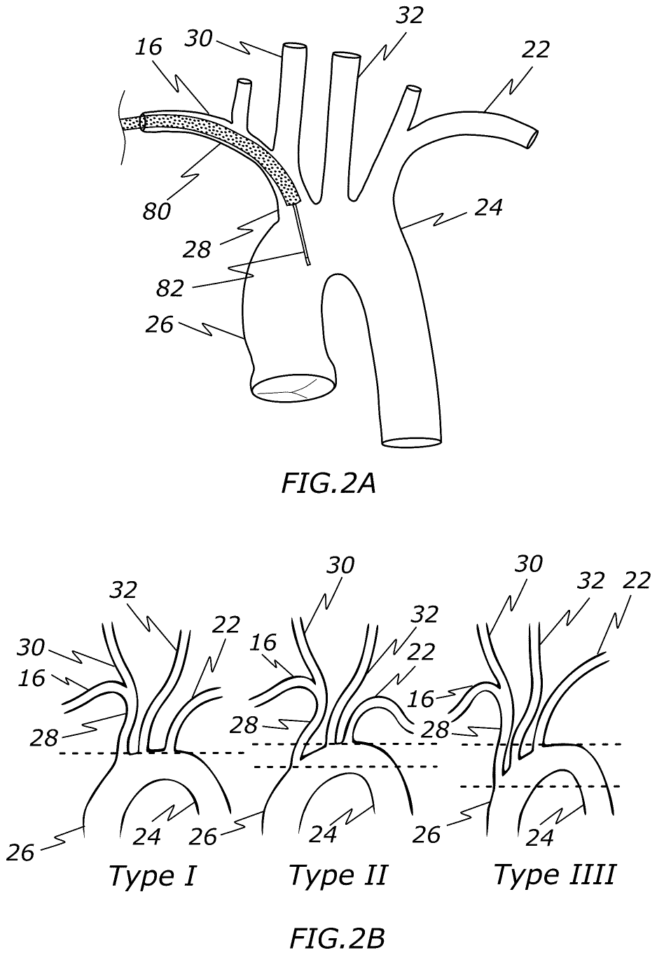

FIG. 2A illustrates a side view of an example aortic arch 24 of a human. As the guide catheter 80 approaches the aortic arch 24 from the right subclavian artery 16 or from the left subclavian artery 22, different arterial pathways may be pursued by a select catheter 82 that is advanced out of the guide catheter 80, depending on the desired target area. In the case of reaching the arteries of the brain, the right common carotid artery 30 may be used to reach the arteries on the right side of the brain and the left common carotid artery 32 may be used to reach the arteries on the left side of the brain.

As seen in FIG. 2A, both common carotid arteries 30, 32 connect at relatively sharp and/or tortuous angles relative to the subclavian arteries 16, 22. Often, many catheters require a significant amount of time to achieve a position such that its distal end can move into either of the common carotid arteries 30, 32. Certain existing catheters may be maneuvered to reach either of the common carotid arteries 30, 32 in some (but not all) patients, but not without great effort and oftentimes requiring the catheter to move against the wall of the brachiocephalic artery 28, the aortic root 26, and/or the aortic valve. In some cases, if the aortic valve or vessel walls are calcified and the catheter exerts a large enough force on the vessel walls of the brachiocephalic artery 28, aortic root 26, and/or the aortic valve, the likelihood of dislodging embolic material increases which can result in a stroke or thrombosis. In other words, existing catheters need to contact or to be “bounced” off the wall of the brachiocephalic artery 28, the aortic root 26, and/or the aortic valve with sufficient force applied to the vessel wall or valve in order to make the required U-turn from the subclavian arteries 16, 22 to the common carotid arteries 30, 32.

FIG. 2B illustrates side views of several different examples of aortic arch 24 anatomies of a human. Navigating the aortic arch 24 as previously described can be further complicated due to different anatomical variations that position the brachiocephalic artery 28 and left common carotid artery 32 in different locations. For example, FIG. 2B illustrates Type I, Type II, and Type III aortic arch structures. Type I aortic arch anatomies tend to be relatively easier to navigate due to the brachiocephalic artery 28, the left common carotid artery 32, and the left subclavian artery 22 being uniformly located along the aortic arch 24, as noted by the dotted line. However, in Type II and Ill aortic arch anatomies, the brachiocephalic artery 28 and the left common carotid artery 32 tend to be located lower and closer to the aortic root 26, creating more abrupt angle changes that must be navigated, as noted by the upper and lower dotted lines in each figure.

As described in further detail below, several example catheters are disclosed with memorized or imparted shapes that are formed when certain regions of the catheter are unrestrained (e.g., when part of the catheter is advanced out of a larger overlying catheter). The imparted shapes of these catheters, often referred to as “select catheters,” may allow a physician to more quickly and efficiently access the right common carotid artery 30 or left common carotid artery 32 from either a right radial access or left radial access approach, as previously described. Further, these select catheters may have a memorized shape that allow a physician to access the right common carotid artery 30 or left common carotid artery 32 without entering the aortic root 26 and/or contacting the aortic valve, which can dislodge embolic material (especially if the aortic valve is calcified) that can lead to a stroke or other complications.

Select catheters may also be referred to as access catheters, inner catheters, support catheters, intermediate catheters, or inner support catheters. A “select catheter” may refer to a catheter whose intended purpose is to assist another catheter (typically a guide catheter) in navigating or selecting challenging anatomy, such as the aortic arch.

In use, the guide catheter may be navigated to a point close to the challenging anatomy, then the “select” catheter may be inserted through the guide catheter lumen and beyond the distal tip of the guide catheter. From there, the “select” catheter may have a biased shape or secondary shape/conformation that it forms once distal to the guide catheter. That secondary shape/conformation may be specific to the challenging anatomy which is being navigated (e.g., Type Ill aortic arch), so that the “select catheter” can be advanced distally into the challenging target vessel. This vessel is thus “selected” by the select catheter. Then, the guide catheter may be advanced over the “select” catheter into the target vessel or a guidewire may be advanced through the select catheter. Once the guide catheter is advanced far enough into the target vessel so that it won't prolapse when the select catheter is removed, then the user may pull the select catheter proximally out of the guide catheter and the procedure may continue with the guide catheter being further advanced to a more distal anatomical target (e.g., aneurysm site, blood clot, etc.).

Generally, the catheters described in this specification may sometimes be referred to as “select” catheters which may be used to navigate certain vascular anatomy of a patient. However, they may also be used for additional purposes during a procedure such as delivering an intravascular device, delivering a bolus of radio-opaque contrast or dye for imaging purposes, aspiration of a clot, and other purposes.

The present specification is directed to several features of a select catheter that may improve its ability to quickly and easily enter a desired vessel. These features may all be included as part of a single example select catheter, or any combination of the features may be included as part of a single select catheter (including use of just a single feature). Generally, these features are directed to the three-dimensional unconstrained shape and a flexibility or stiffness profile of the select catheter.

One such feature comprises a distal region of the select catheter that curves in at least two different dimensions when unconstrained from an outer catheter (i.e., the curvature does not remain substantially in a single plane). In one example, a distal region of the select catheter comprises a first distal section that forms a major curve with a second distal section. The first distal section is distal of a second distal section and forms a major curve with the second distal section when unconstrained. The second distal section lies substantially in a first plane and the first distal section is positioned at least partially outside of the first plane and such that a free distal tip of the distal section is positioned proximally of the major curve. Put another way, the major curve positions the free distal tip in a generally proximal direction and in an orientation that the first distal section is not parallel to the second distal section. In another example, the phrase “proximally of the major curve” may mean that the distal tip is located closer to the proximal end of the catheter and the major curve is located distally with respect to the distal tip. Additional details of the select catheter are discussed later in this specification.

Typically, existing select catheters only curve within or remain substantially within a single plane which can make accessing some vessels in an aortic arch difficult, particularly for Type II and Type Ill anatomies as previously discussed with regard to FIG. 2B. By further including curvature along the distal region of the inner catheter that does not remain within a single plane (e.g., a major curve that positions the free distal tip of the catheter in a generally proximal direction while positioning a distal most region in a non-parallel orientation to proximal adjacent regions of the select catheter), different vessels can be more easily accessed, particularly for Type II and Type III anatomies. For example, the right common carotid artery 30 and the left common carotid artery 32 can be more easily accessed by a select catheter comprising a distal region that curves in at least two different dimensions.

Additionally, a region distal of the major curve may further include a minor curve (i.e., a curve with less curvature than the major curve). The minor curve may curve at orientations, angles, and/or planes similar to the major curve (e.g., both major and minor curves may be substantially positioned in the same plane) or may curve at orientations, angles, and/or planes different than that of the major curve (e.g., both major and minor curves may be substantially positioned in the different planes).

In another example, the select catheter comprises a stiffness profile that provides certain stiffnesses at certain distances from its distal tip that helps prevent loss of access of the select catheter when tracking a guidewire or larger catheter through/over it. For example, some existing select catheters include a relatively large jump in stiffness at about 8 cm from their distal tips, which can result in the aforementioned loss of access (i.e., the select catheter tends to pull out of a desired vessel it is initially positioned in). By providing a less abrupt transition, the select catheter may better maintain access to or position in a desired vessel during a procedure.

In another example, the size of the unconstrained shape, the flexibility of the unconstrained shape, and/or the curvature of the unconstrained shape may allow the select catheter to form its unconstrained shape within an aortic arch 24 of a patient without the need to contact walls of the aortic root 26 or the aortic valve or with only a minimal force applied to the vessel wall or aortic valve (low enough force such that dislodgment of plaque is highly unlikely). Some existing select catheters have a two-dimensional secondary shape that requires the select catheter to be pressed against or “bounced” off of the aortic root 26 and/or the aortic valve with sufficient force applied to the vessel wall or valve in order to make the required U-turn from the subclavian arteries 16, 22 to the common carotid arteries 30, 32, which may dislodge embolic material. In the present example, the ability to substantially reduce or avoid this aortic contact (and corresponding forces on the vessel wall or valve) may reduce or avoid the risk of dislodging embolic material and therefore may reduce the risk of strokes, thrombosis, or similar complications during or after a procedure.

Typically, the select catheters of this specification intended for radial access have a length long enough to at least allow it to travel from entry at the subclavian arteries 16, 22 and to extend near or into either of the common carotid arteries 30, 32. However, longer lengths that allow further advancement into either of the common carotid arteries 30, 32 are also possible.

As previously discussed, the select catheters of this specification may have memorized, secondary, unconstrained, or imparted shapes at least along a distal portion of the catheter. Such memorized shapes may be achieved in several different ways. For example, a shape memory alloy (e.g., Nitinol) that has a heat-set memorized shape at certain temperatures can be used in at least the distal portion of the select catheter. For example, the shape memory alloy may take the form of a braided wire layer, a coiled wire layer, or similar variations and combinations. Often, such a shape memory alloy layer may be disposed between at least an inner and outer polymer layer which may further impact both shape and stiffness.

The select catheters of this specification generally include a distal portion which may extend from about the distal tip of the select catheter to a location proximal of the distal tip. For example, the distal portion may have a length within an inclusive range of about 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, 20 cm, and values in between and beyond. Depending on the memorized (or imparted) shape of the distal portion and which arterial pathway the select catheter is intended to take, the length of the distal portion may vary.

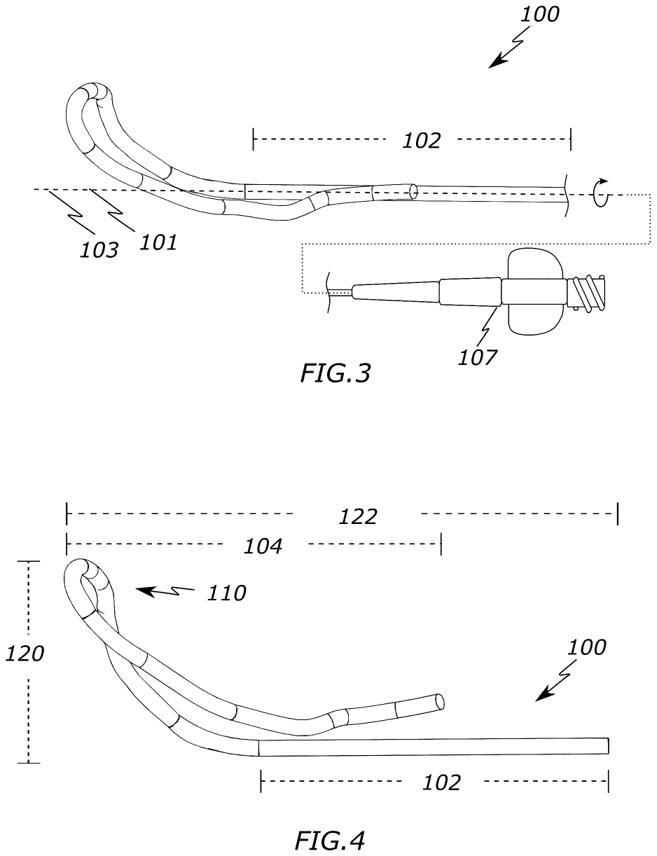

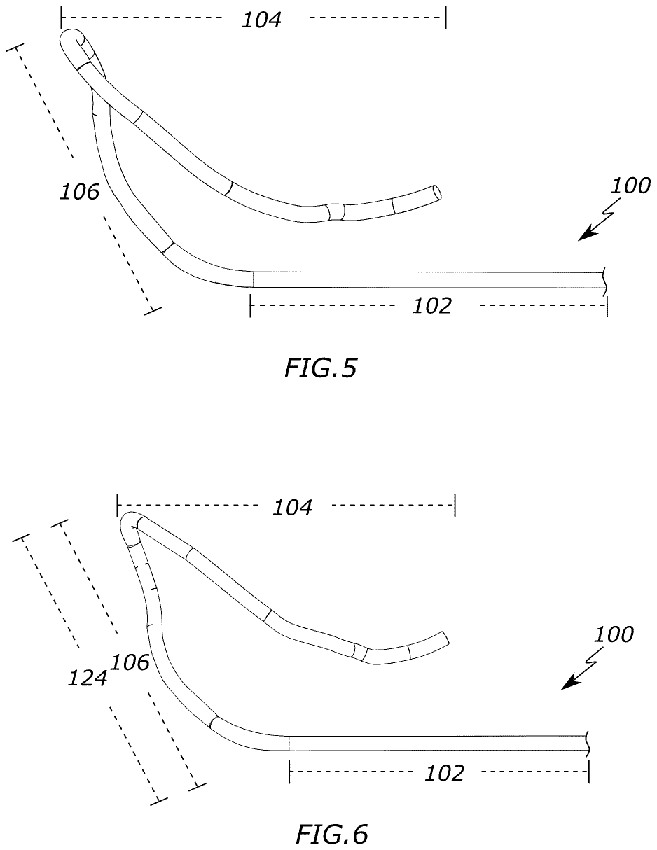

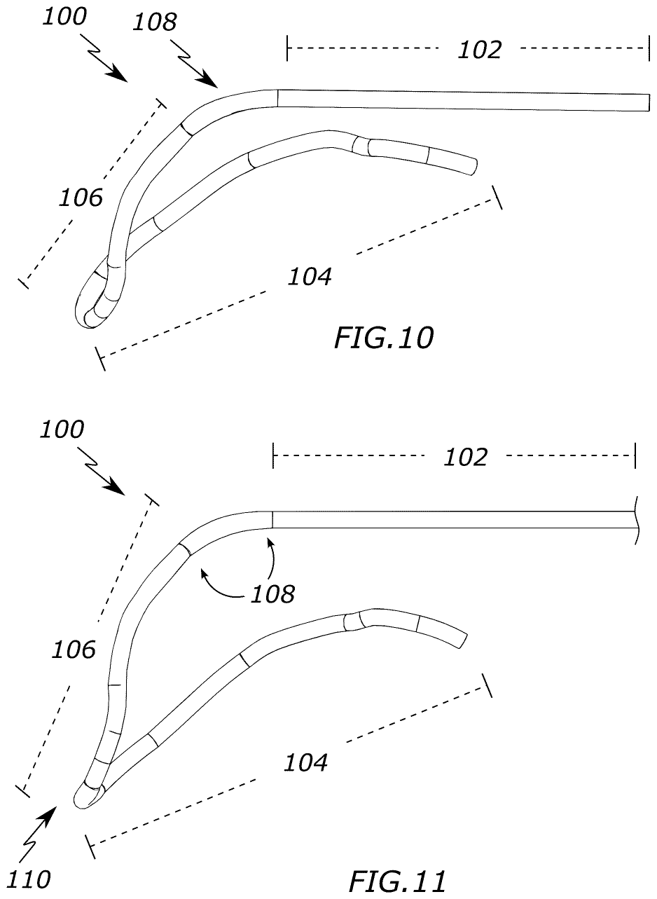

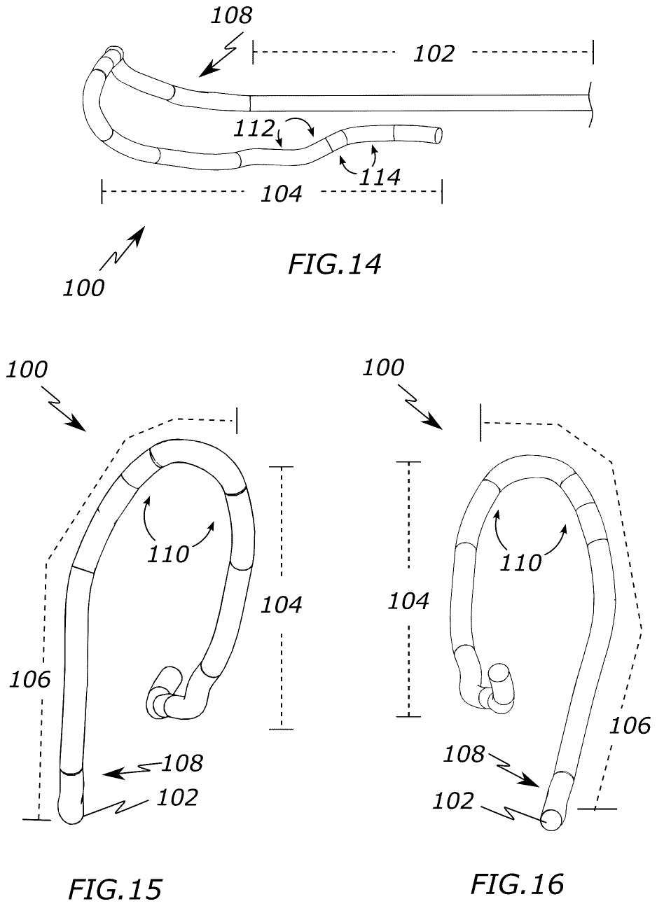

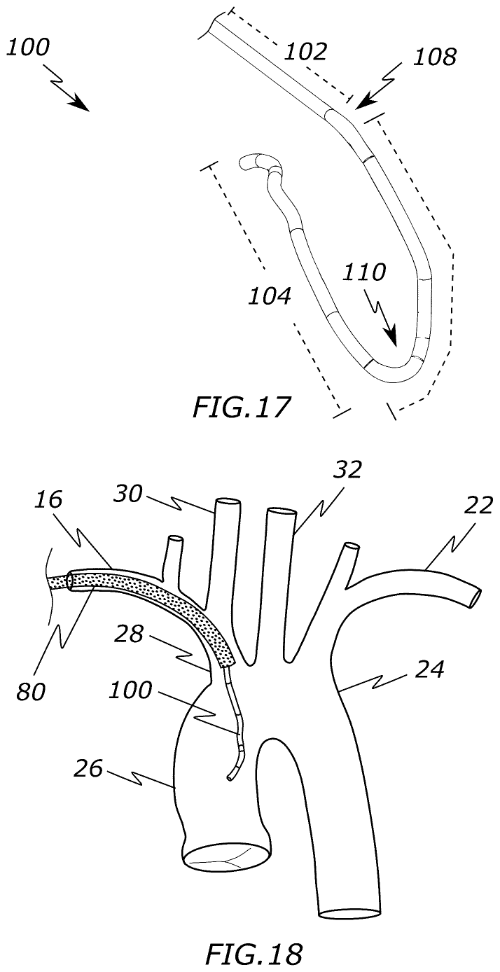

FIGS. 3-17 illustrate a distal portion of a select catheter 100 for access to the left common carotid artery 32 from the right subclavian artery 16 (i.e., a right radial access approach). In some examples, the unconstrained or memorized shape of the select catheter 100 may include several different curves that create a shape that lies substantially outside of a single plane. Put another way, the curves of this memorized shape select catheter may not all curve within only X-Y dimensions (e.g., 2D coordinate system), but instead may curve with X-Y-Z dimensions (e.g., 3D coordinate system). To help illustrate the complexity of these curvatures, FIGS. 3-14 illustrate a distal portion of the catheter 100 at various rotational orientations that have been rotated relative to an axis 101 (see FIG. 3) that extends through a straight region 102 of the distal portion and the remaining proximal portion (not shown in FIG. 3). Note that the arrow of axis 101 indicates the direction of rotation (clockwise if looking from a proximal end of the axis or catheter 100).

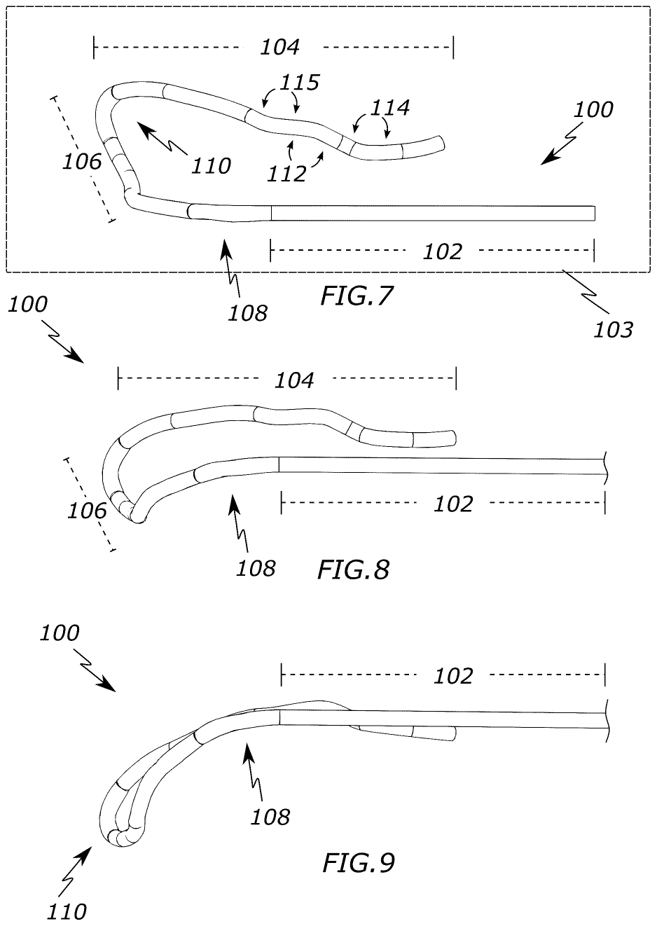

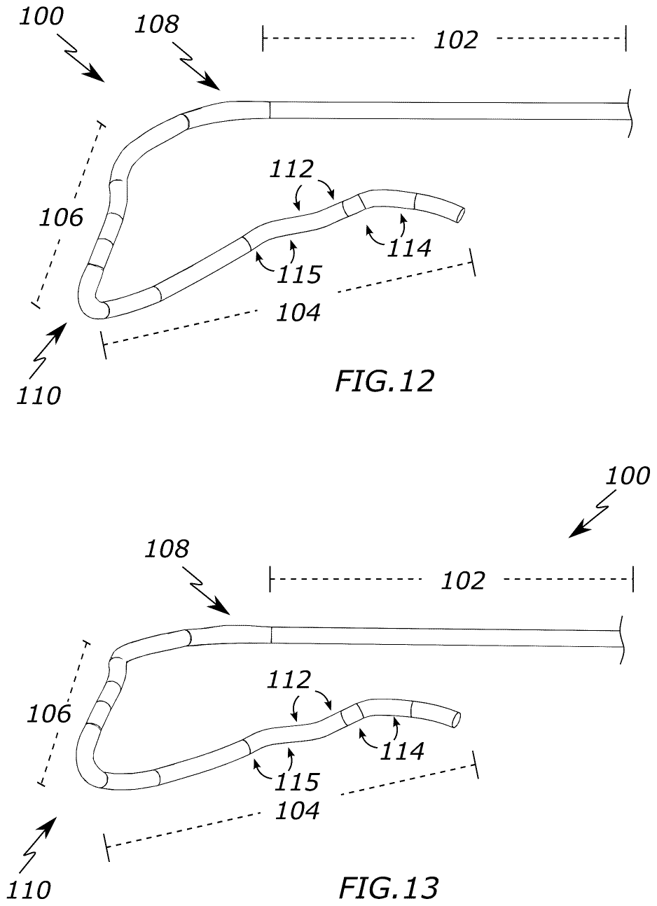

Generally, the distal portion of the select catheter 100 forms an unconstrained or memorized shape having a “U” shape, as seen best in the rotational orientation of FIGS. 12 and 13. However, in some examples, the “U” shape is also curved or bent in a roughly perpendicular direction to the front of the “U” shape (e.g., the bottom of the “U” bends outward towards the viewer). Put another way, a plane 103 is illustrated in FIGS. 3 and 7 in views that are generally perpendicular to each other. The “U” shape is seen best in FIG. 7 and tends to bend out of or away from the plane 103, which is best seen in FIG. 3. When this general shape is formed within the vasculature (e.g., within the aortic arch 24), the select catheter 100 can be proximally pulled upwards such that the distal end of the select catheter 100 enters the left common carotid artery 32.

The memorized shape of the distal portion of the select catheter 100 may be generally described as having three lengths or regions of the distal portion. A proximal region 102, an intermediate region 106, and a distal region 104.

Referring to FIGS. 7-9, in some examples, the proximal region 102 is generally straight and is joined at its distal end to a proximal end of the intermediate region 106 via a bend or curve 108 (note, proximal and distal here refers to the proximal and distal locations when the select catheter is in a restrained, linear shape (e.g., within another catheter) and not relative to other regions when in its unconstrained, memorized shape). A distal end of the intermediate region 106 is connected via a bend or curve 110 to a proximal end of the distal region 104. In some examples, the curves 108 may be considered their own separate regions or may be part of the regions 102, 104, 106, however, these should be considered as only different approaches to a description of a unitary shape of the distal portion.

In the present example, the curve 108 generally curves in a first direction and the curve 110 curves in a second direction, different from the first direction. For example, the curve 110 may generally curve at about a perpendicular orientation (e.g., about 90 degrees) relative to the curve 108. Note that “generally” and “about” are used throughout this specification to mean a plus-or-minus variation/range of 15% in any direction and/or value. This is particularly true since the intermediate region 106 and the distal region 104 may not be perfectly straight and therefore may have further curvatures and directions of curvatures imparted to them.

As seen in the view of FIG. 11, in one example, the curve 108 has an angle of curvature within an inclusive range of about 35 degrees to about 75 degrees. For example, 35, 45, 55, 65, 75 degrees and values within those angles. In one specific example, the curve 108 has an angle of curvature of about 55 degrees.

As seen in the view of FIGS. 15 and 16, in another example, the curve 110 has an angle of curvature within an inclusive range of about 160 degrees to about 200 degrees. For example, 160, 170, 180, 190, 200 degrees and values within those angles. In one specific example, the curve 110 has an angle of curvature of about 180 degrees. In some examples, curve 110 may be composed of a single curve with a single curvature, or areas of different curvature. For example, as seen in FIGS. 15 and 16, the curve 110 starts on the left, proximal portion as having a gentle curve in one area and a more abrupt curve in a more distal area.

In one example, the proximal region 102 has a length within an inclusive range of about 20 mm to about 50 mm. For example, 20, 25, 30, 35, 40, 45, 50 mm and lengths in between those values. In one specific example, the length is about 38 mm.

In one example, the intermediate region 106 has a length in its expanded shape within an inclusive range of about 15 mm to about 40 mm. For example, 15, 20, 25, 30, 35, 40 mm and lengths in between those values. In one specific example, the length is about 27 mm.

In one example, the distal region 104 has a length in its expanded shape within an inclusive range of about 25 mm to about 65 mm. For example, 25, 30, 35, 40, 45, 50, 55, 60, 60 mm and lengths in between those values. In one specific example, the length is about 45 mm.

In its unconstrained, expanded, memorized shape, the distal portion of the select catheter 100 has dimensions within the following inclusive ranges, including a length 122 (FIG. 4) of about 3.0 cm to 8.5 cm, width 124 (FIG. 6) of about 1.5 cm to 3.0 cm, and height 120 (FIG. 4) of about 1.5 cm to 3.5 cm.

As seen best in FIGS. 12-14, in some examples, the distal region 104 may also include a plurality of minor curves 112, 114, and 115 (i.e., curves with a smaller angle than those of curves 108, 110). For example, these curves may be within an inclusive range of about 5 degrees to about 90 degrees and may curve in different planes from each other. Alternatively, in some examples, the curves 112, 114, and 115 may be relatively larger, being within an inclusive range of about 90 degrees to 180 degrees. In another example range, the curves are within an inclusive range of about 5 degrees to 30 degrees. In some examples, distally beyond the curves 112, 114, and 115, a relatively straight segment may be included having an inclusive range within 5 mm to 16 mm in length. Although not shown, select catheter 100 may exclude one or more minor curves 112, 114, and 115 at the intermediate region 106 and/or the distal region 104.

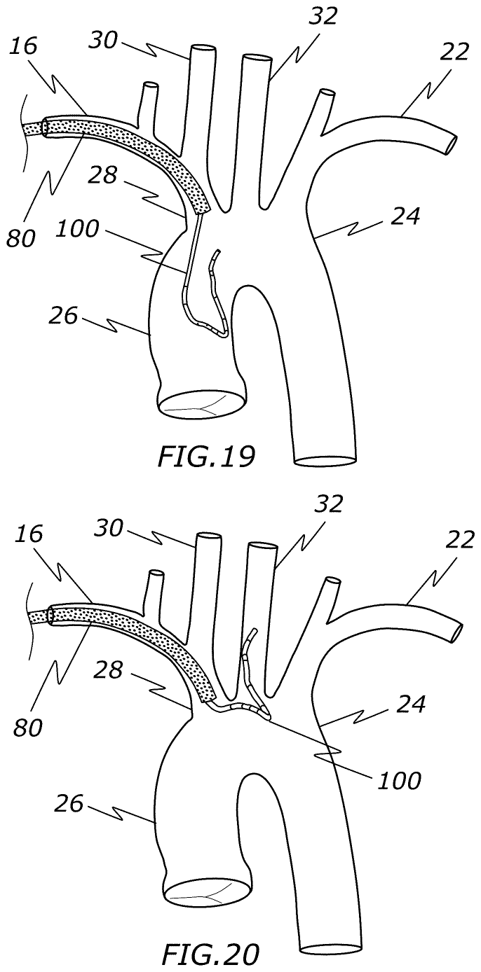

FIGS. 18-20 illustrate various views of a method of using the select catheter 100 of FIGS. 3-17. In one example method of use seen in FIGS. 18-20, a guide catheter 80 is advanced via a right radial approach (as previously described) such that its distal end is located in or near the brachiocephalic artery 28 (alternatively, in the right subclavian artery 16 or the aortic arch 24). Next, as seen in FIGS. 18 and 19, the distal portion of the select catheter 100 is advanced out of the guide catheter 80 and into the aortic arch 24 such that it takes on its unconstrained, expanded, memorized shape. Once the memorized shape has been achieved, a physician may proximally retract the select catheter 100 such that some or all of the proximal portion 102 is back within the guide catheter 80. Since the intermediate region 106 is sized and shaped similar to the distance and location of the left common carotid artery 32, the proximal movement moves the distal region 104 into the left common carotid artery 32 as illustrated in FIG. 20.

Depending on the physician's use, the select catheter 100 may then be further distally advanced to move the distal portion of the catheter 100 further up the left common carotid artery 32, the guide catheter 80 may be advanced over the select catheter 100 into the left common carotid artery 32, or variations and combinations of each may be performed. Depending on the plan of a physician, the select catheter 100 may be removed and additional working catheters (or guidewires) may be used for an intended treatment.

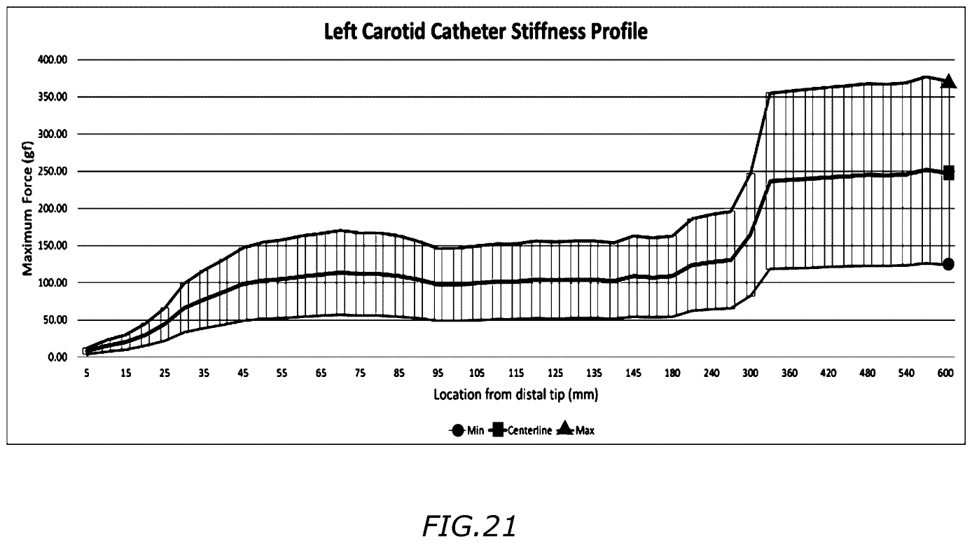

FIG. 21 illustrates a chart of a stiffness profile of the select catheter 100 of FIGS. 3-17. In addition to the unconstrained, expanded, memorized shape of the distal portion of the select catheter 100, the select catheter 100 may have certain stiffnesses at certain lengths from its distal tip, as seen in FIG. 21. In one example stiffness aspect, the stiffness generally of the proximal region 102 may be less, on average, than that of the intermediate region 106. The relatively reduced stiffness of the proximal region 102 may provide additional flexibility for the portion of the catheter within the tortuous right subclavian artery 16 and therefore may better allow the remaining regions to reach the left common carotid artery 32 or left subclavian artery 22. For example, a distal region 104 of about 45 mm may increase in the amount of force needed for deflection between about 0 gf to 100 gf (plus or minus 50 gf), an intermediate region 106 of about 27 mm may increase in the amount of force needed for deflection between about 100 gf to 120 gf (plus or minus 50 gf), and a proximal region 102 of about 38 mm may generally decrease in the amount of force needed for deflection between about 120 gf to 100 gf (plus or minus 50 gf).

Variations in the mechanical design of the select catheter 100 may include but not limited to a combination of polymer jacket durometers ranging from 25D to 74D, wall thickness from 0.004 inch to 0.011 inch, and/or the use of additional support liners such as PTFE, Filmcast, and/or Pebax, metal coil(s) (0.003 inch-0.018 inch pitch) and/or braid patterns (40-120 PPI) composed of Stainless Steel, Tungsten, Platinum, Tantalum, Nitinol, drawn filled tubing (DFT) Nitinol that are strategically placed around the curvatures of the shape and proximal end of the catheter for optimized shape retention, pushability, torqueability/torque response, kink resistance, support and trackability. In some examples, the braiding wire can be round (ranging 0.001-0.003 inch diameter) or flat wire (ranging from 0.001×0.003 to 0.002×0.005 inch) composed of Stainless Steel, Nitinol, drawn filled tubing (DFT) Nitinol. For increased trackability, in some examples, a siloxane based slick additive can be added to the polymer extrusion. For improved radiopacity under fluoroscopic interventions, in some examples, barium, tungsten, or other materials may be added to the polymer extrusion. In some examples, a marker band of any combination of iridium, barium, tungsten, platinum, gold, or other radiopaque materials can also be added for fluoroscopic visibility.

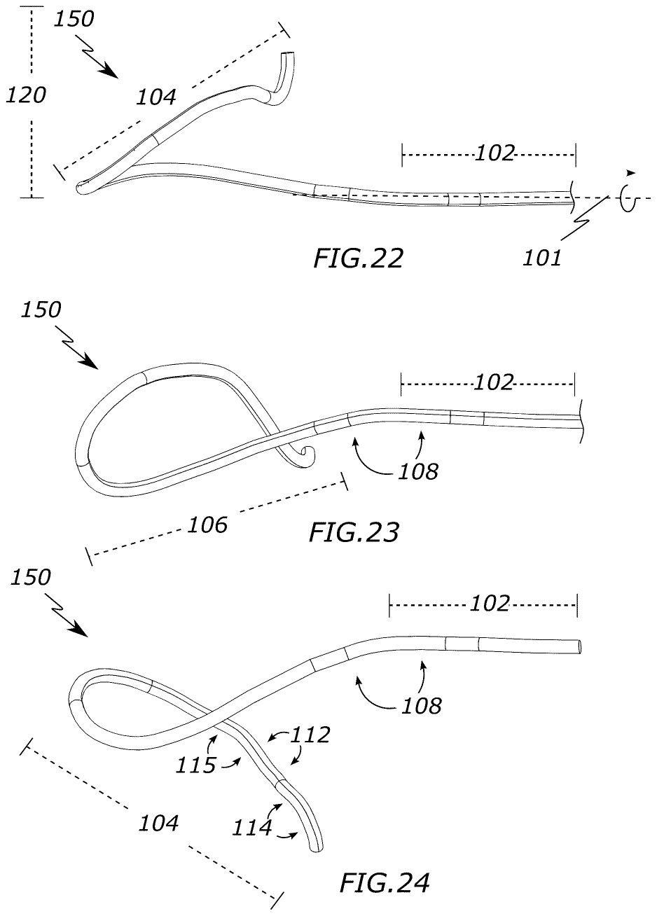

FIGS. 22-28 illustrate another example of a select catheter 150 that is generally similar to the previously described select catheter 100, including stiffness, except with some minor differences in angles of curvature and sizes.

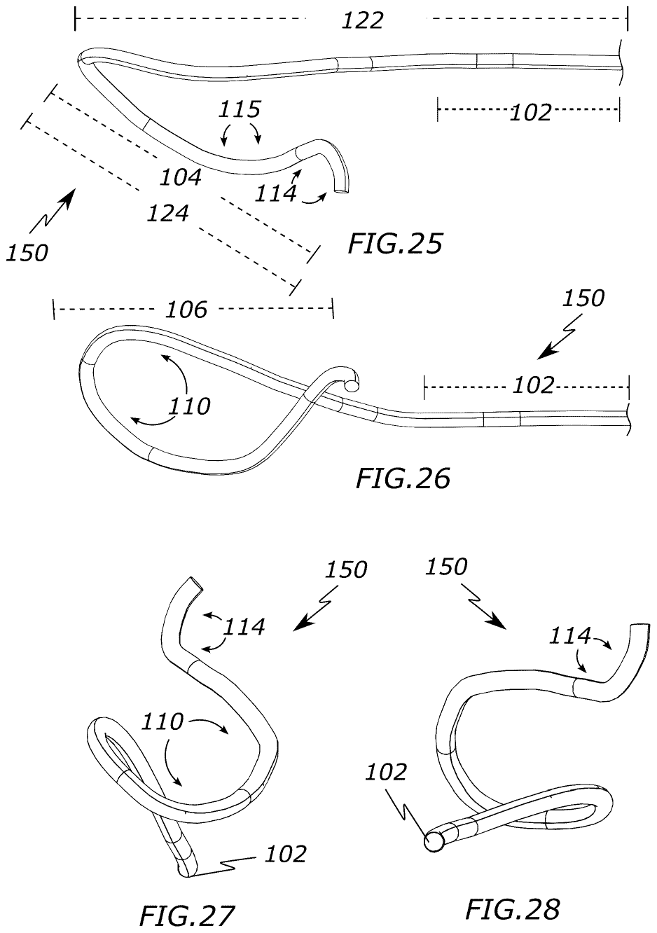

In another example, the curve 115 has a somewhat larger curve as seen in FIGS. 24 and 25. For example, the curve 115 has an angle or curvature within an inclusive range of about 25 degrees to about 65 degrees, such as 25, 30, 35, 40, 45, 50, 55, 60, 65 degrees, or angles in between these values. In one specific example, the curve 115 has a curvature of about 45 degrees.

In another example, the curve 114 has a somewhat large curve as seen in FIGS. 24 and 25. For example, the curve 114 has an angle or curvature within an inclusive range of about 200 degrees to about 300 degrees, such as 200, 210, 220, 230, 240, 250, 260, 270, 280, 290, 300 degrees, or angles in between these values. In one specific example, the curve 115 has a curvature of about 250 degrees.

In one example, the proximal region 102 has a length within an inclusive range of about 10 mm to about 40 mm. For example, 10, 15, 20, 25, 30, 35, 40 mm and lengths in between those values. In one specific example, the length is about 20 mm.

In one example, the intermediate region 106 has a length in its expanded shape within an inclusive range of about 50 mm to about 90 mm. For example, 50, 55, 60, 65, 70, 75, 80, 85, 90 mm and lengths in between those values. In one specific example, the length is about 67 mm.

In one example, the distal region 104 has a length in its expanded shape within an inclusive range of about 50 mm to about 90 mm. For example, 50, 55, 60, 65, 70, 75, 80, 85, 90 mm and lengths in between those values. In one specific example, the length is about 67 mm.

In some examples, in its unconstrained, expanded, memorized shape, the distal portion has dimensions within the following inclusive ranges, including a length 122 (FIG. 25) of about 7 to 10 cm, width 124 (FIG. 25) of about 1.5 cm to 3.0 cm, and height 120 (FIG. 22) of about 1.5 cm to 3.5 cm.

In another specific example, the length between curve 115 and curve 114 is within an inclusive range of about 1.0 cm and 3.5 cm, the length between curve 115 and curve 110 is within an inclusive range of about 1.5 cm and 3 cm, and the length between curve 110 and curve 108 is within an inclusive range of about 2.0 cm and 5.0 cm.

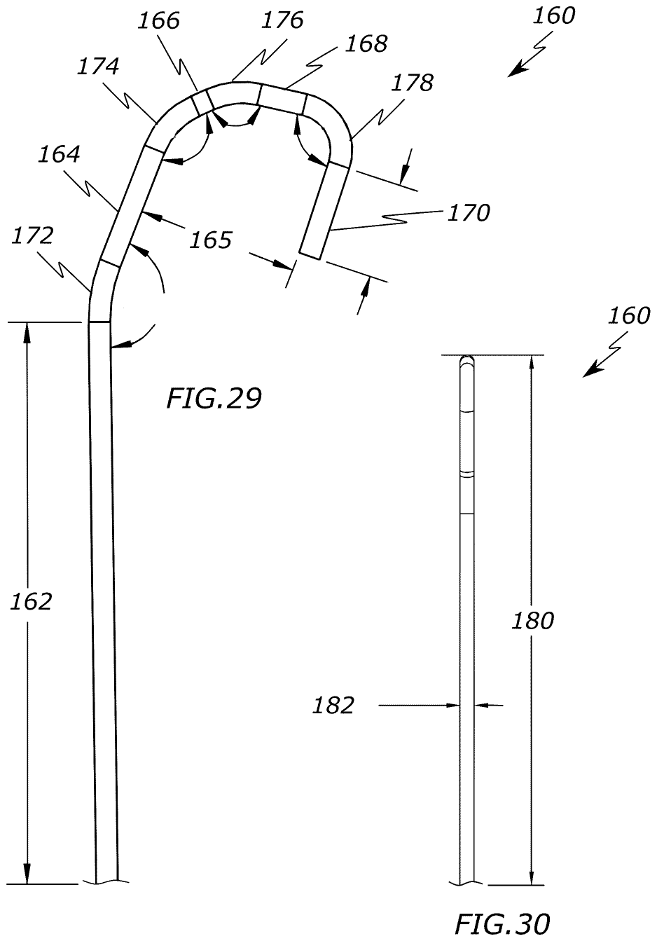

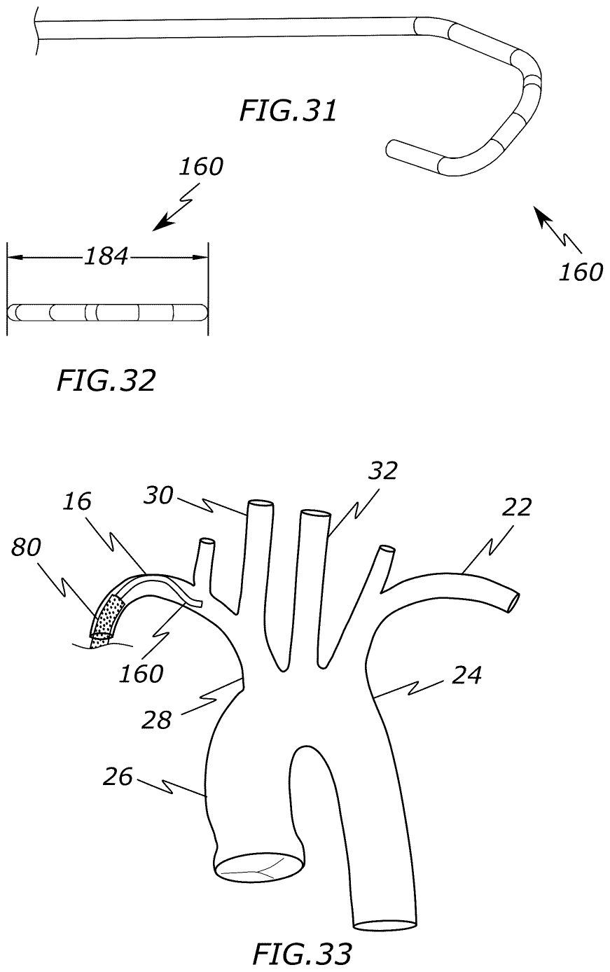

FIGS. 29-32 illustrate a distal portion of a select catheter 160 for access to the right common carotid artery 30 from the right subclavian artery 16 (i.e., a right radial access approach). In some examples, the unconstrained or memorized shape of the select catheter 160 may include several different curves that create a shape that lies generally within a single plane.

The distal portion of the select catheter 160 may include several generally straight segments that are connected to each other via a plurality of curved segments. The straight segments include proximal segment 162, first intermediate segment 164, second intermediate segment 166, third intermediate segment 168, and distal segment 170. These segments may have lengths within the following inclusive ranges. Proximal segment 162: 4-10 cm, first intermediate segment 164: 0.5-1.5 cm, second intermediate segment 166: 0.1-0.5 cm, third intermediate segment 168: 0.5-1.0 cm, and distal segment 170: 0.5-1.0 cm.

In one example, the distal portion of the select catheter 160 may have a length 180 within an inclusive range of about 1.5 cm to 6.5 cm, a width 184 within an inclusive range of about 1 cm to 3.5 cm, an outer diameter 182 within an inclusive range of about 1.700 mm to 1.778 mm, and a length of separation 165 of its distal tip of within an inclusive range of about 0.5 cm to 2.5 cm. Hence, in one example, the total width of the “U” shape of the distal portion without regard to curve 172 and proximal region 162 is within an inclusive range of about 0.7 cm to about 2.9 cm.

In one example, the first curve 172 between segments 162 and 164 may have an angle of curvature within an inclusive range of about 70 degrees to 140 degrees. The second curve 174, third curve 176, and fourth curve 178 may have angles of curvature within an inclusive range of about 50-140 degrees. In one specific example, the first curve 172 may have an angle of curvature of about 158 degrees, the second curve 174 may have an angle of curvature of about 135 degrees, the third curve 176 may have an angle of curvature of about 143 degrees, and the fourth curve 178 may have an angle of curvature of about 85 degrees.

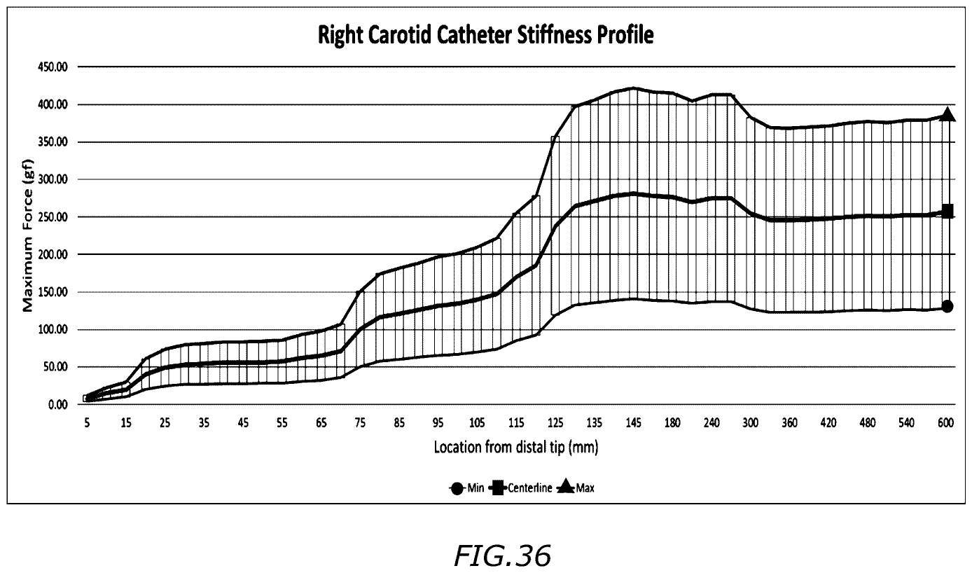

In addition to the unconstrained, expanded, memorized shape of the distal portion of the select catheter 160, it may also have certain stiffnesses at certain lengths from its distal tip, as seen in FIG. 36. For example, segment 170 and curve 178 may have an average stiffness increasing from about 0 gf to about 15 gf (plus or minus 10 gf) while the remaining segments and curves 168, 176, 166, 174, 164, 172, and 162 may have an average stiffness within an inclusive range of about 15 gf to about 75 gf (plus or minus 50 gf).

The select catheter 160 may utilize strategic internal and external material transitions around the curvature of the shape. Variations in the mechanical design of the catheter 160 may include a combination of polymer jacket durometers ranging from 25D to 74D, wall thickness from 0.004″ to 0.011″. Nylon may be included on the proximal end in some embodiments while the distal end contains softer durometers. In one example, the use of additional support liners such as extruded etched PTFE, Filmcast PTFE, and/or Polyamide, metal coil(s) (0.003″-0.018″ pitch) composed of Stainless Steel, Tungsten, Platinum, Tantalum, Nitinol, drawn filled tubing (DFT) Nitinol, and braid patterns (40-120 PPI) may be strategically placed around the curvatures of the memorized shape. These transitions may aid in the optimized shape retention, push ability, torqueability/torque response, kink resistance, support and trackability. Braiding wire can be round (ranging 0.001-0.003 inch diameter) or flat wire (ranging 0.001×0.003 to 0.002×0.005 inch) composed of Stainless Steel, Nitinol, drawn filled tubing (DFT) Nitinol. For increased trackability, a siloxane based slick additive may be added to the polymer extrusion. For improved radiopacity under fluoroscopic interventions, barium, tungsten, or other materials may be added to the polymer extrusion.

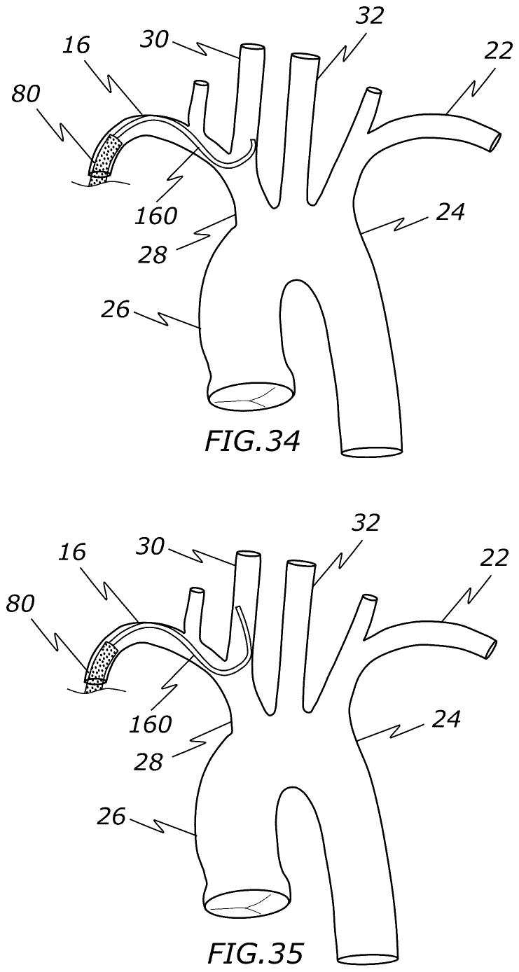

In one example method of use seen in FIGS. 33-35, a guide catheter 80 is advanced via a right radial approach (as previously described) such that its distal end is located in the right subclavian artery 16. In FIG. 33, the select catheter 160 is advanced out of the guide catheter 80 such that it engages with the inferior section of right subclavian artery 16 and an upward force orients the select catheter tip angle upwards. As the distal portion of the select catheter 160 is further distally advanced, as seen in FIG. 34, the tip of the select catheter 160 curves upward towards the right common carotid artery 30. As seen in FIG. 34, further distal movement causes the distal tip to move into the right common carotid artery 30 while the proximal curvatures and position of the distal portion force the distal portion to remain engaged to the lumen wall of the right subclavian artery 16 to help maintain stability. In short, the shape of the distal portion of the select catheter 160 contacts and maintains contact with the walls of the right subclavian artery 16 while also causing the distal tip to move into the right common carotid artery 30.

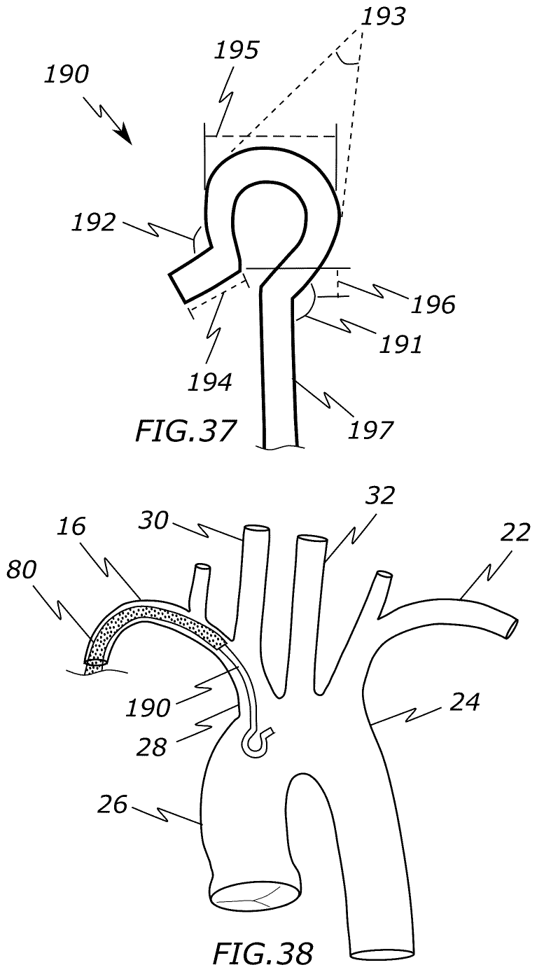

FIG. 37 illustrates another example of a select catheter 190 that is similar to the previously described select catheter 160, including stiffness, but has a different unconstrained, memorized shape of its distal portion for accessing the right common carotid artery 30 from the right subclavian artery 16. Generally, the memorized shape forms a primary curve with an elongated distal portion extending therefrom.

Turning first to the angles shown in FIG. 37, the three angles 191, 912, and 193 may be within an inclusive range of about 10 degrees to 150 degrees. In a more specific example, angle 191 may be within an inclusive range of about 100 degrees to about 150 degrees, angles 192 may be within an inclusive range of about 30 degrees to about 125 degrees, and angle 193 may have an angle of curvature within an inclusive range of about 10 degrees to about 25 degrees (or an inner diameter of about 335 degrees to about 350 degrees) and also having a diameter of the curve range from about 0.5 cm to 1.5 cm. While the above example angles may be generally within the same plane, distal portion may also include additional curvature angles between about 0 and 90 degrees in a generally perpendicular direction to the curves shown in FIG. 37.

In another example, each of the segments 194, 195 (length and width in its curved state), and 197 may be within an inclusive range of about 0.5 cm to about 2.5 cm in length.

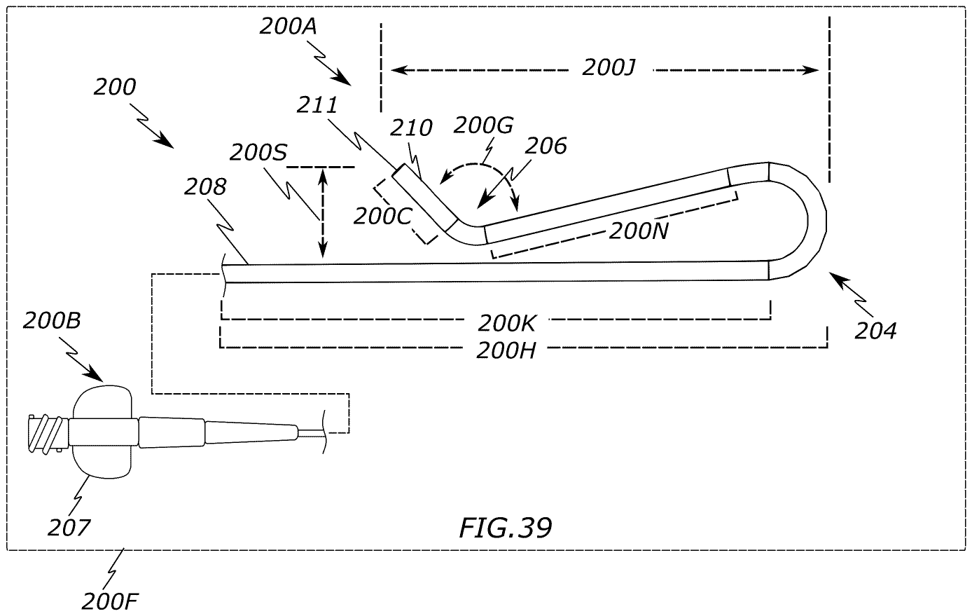

FIG. 38 illustrates an example method of use in which a guide catheter 80 is advanced via a right radial approach (as previously described) such that its distal end is located in or near the brachiocephalic artery 28 (alternatively, in the right subclavian artery 16 or the aortic arch 24). Next, the distal portion of the catheter 190 is advanced out of the guide catheter 80 and into the aortic arch 24 such that it takes on its unconstrained, expanded, memorized shape. Once the memorized shape has been achieved, a physician may proximally retract the select catheter 190 such that at least the distal segment 194 moves into the right common carotid artery 30.

Depending on the physician's use, the select catheter 190 may then be further distally advanced to move the distal portion of the catheter 190 further up the right common carotid artery 30, the guide catheter 80 may be advanced over the select catheter 190 into the right common carotid artery 30, or variations and combinations of each may be performed. Depending on the plan of a physician, the select catheter 190 may be removed and additional working catheters may be used for an intended treatment.

As previously discussed, existing select catheters only curve within or remain substantially within a single plane which can make accessing some vessels in an aortic arch difficult, particularly for Type II and Type III anatomies as previously discussed with regard to FIG. 2B. By including curvature along the distal tip of the select catheter so that at least some of a distal region of the select catheter does not remain within a single plane (e.g., a major curve that positions the free distal tip of the catheter in a generally proximal direction while positioning a distal most region in a non-parallel orientation to proximal adjacent regions of the select catheter), different vessels can be more easily accessed. For example, the right common carotid artery 30 and the left common carotid artery 32 can be more easily accessed.

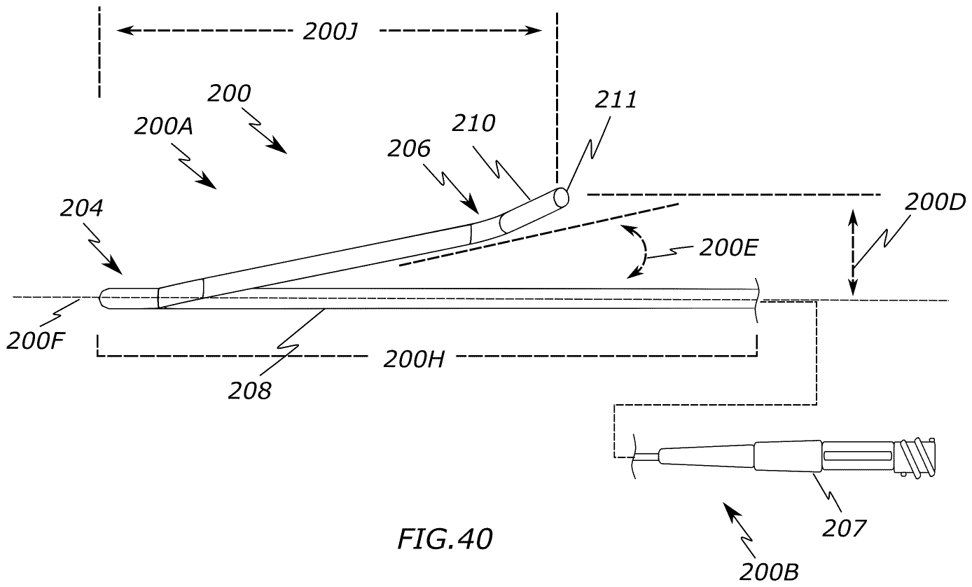

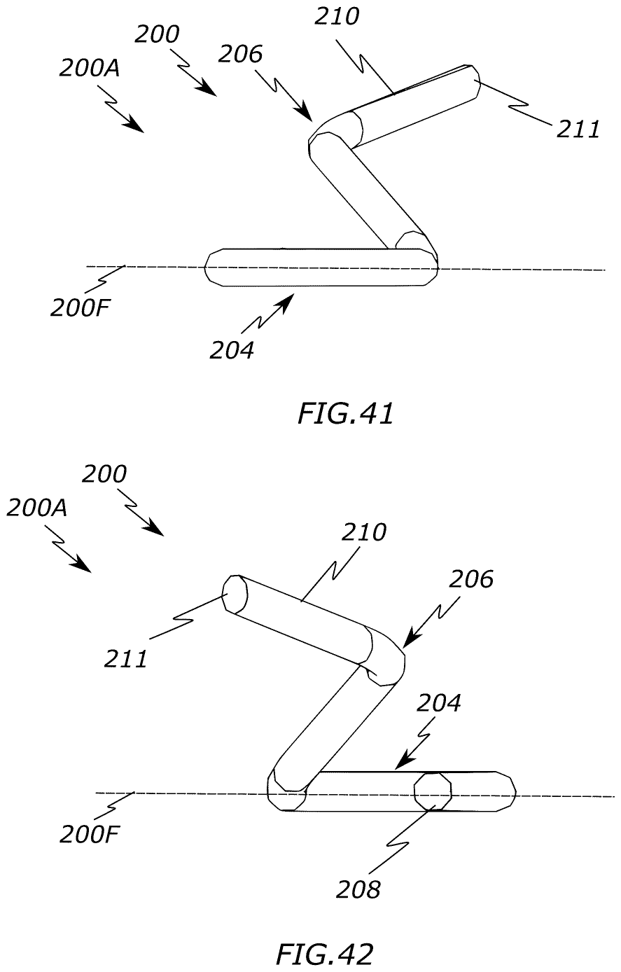

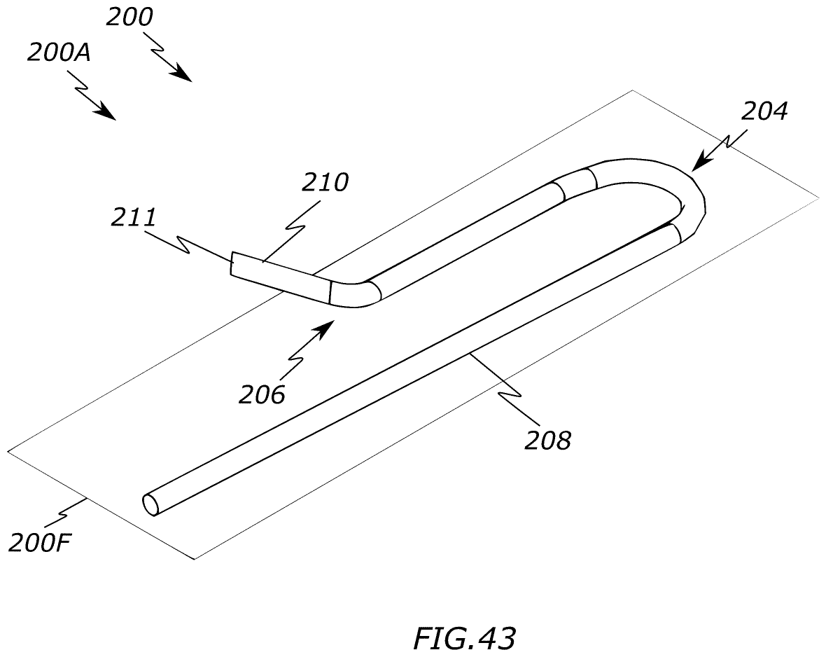

FIGS. 39-43 illustrate an example of a select catheter 200 with a curved distal region. FIG. 39 illustrates a side view of the select catheter 200 in a first rotational position relative to a plane 200F. FIG. 40 illustrates a side view of the select catheter 200 in a second rotational position that is about perpendicular to the position shown in the view of FIG. 39. FIG. 41 illustrates an end view of the select catheter 200 from distal end perspective. FIG. 42 illustrates an end view of the select catheter 200 from a proximal end perspective. FIG. 43 illustrates a perspective view of the select catheter 200. The reference plane 200F is included in each of these figures to help convey the shape of the select catheter 200. In FIG. 39, the face of the reference plane 200F faces the viewer and in FIG. 40 the edge of the reference plane 200F faces the viewer.

In one example, the select catheter 200 may comprise a generally elongated shape or body that may have a lumen therethrough (e.g., a guidewire lumen) or may not have a lumen. In some examples, the select catheter 200 may include a variety of different structural materials that impart different flexibilities, such as inner wire coils, inner braided wires, and outer polymer jackets of different durometer.

Returning to the example of FIG. 39, the select catheter 200 may include a distal region 200A and a proximal region 200B. The proximal region 200B may include a proximal catheter hub 207 or a similar terminal end that allows connection to other medical devices and/or access to the interior lumen, if present. The distal region 200A may extend a distance from the proximal region 200B and may terminate at a distal tip 211 of the select catheter 200. As described further below, the distal region 200A includes an imparted secondary shape that forms when at least part of the distal region 200A is unconstrained (e.g., not constrained by a larger outer catheter). In one example, the distal region may have a length within an inclusive range of about 3 cm and about 30 cm when straightened.

In one example, this distal region 200A may include a distal first section 210 (also denoted by length 200J) and a proximal second section 208 (also denoted by length 200H). The distal first section 210 and the proximal second section 208 may be unitary with each other where the distal first section 210 is distal and connected to the proximal second section 208 when the select catheter 200 is in a constrained linear shape. Adjacent ends of the distal first section 210 and the proximal second section 208 form a first major curve 204 with each other. In one example, a major curve may be defined as either the largest curve when the distal region 200A is unconstrained or a curve that bends beyond 45 degrees.

As seen in FIGS. 39 and 40, the first major curve 204 is formed with adjacent ends of the distal first section 210 and the proximal second section 208 and which does not remain substantially in a single plane 200F. In one example, as can be seen in FIG. 39, the first major curve 204 may curve back in a generally proximal direction of about 193 degrees when viewed against the face of the reference plane 200F or a radius of curvature of about 0.170 inch. However, a variety of different angles may also be possible, such as an inclusive range of 150 degrees to 230 degrees. In more specific examples, the following may be possible: 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230 degrees. Example radius of curvature include an inclusive range of 0.14 inch to 0.30 inch, or more specifically: 0.14, 0.15, 0.16, 0.17, 0.18, 0.19, 0.20, 0.21, 0.22, 0.23, 0.24, 0.25, 0.26, 0.27, 0.28, 0.29, 0.30 inch. Hence, the tip 211 of the select catheter 200 is positioned generally proximally of the first major curve 204 when the distal region 200A is unconstrained. Put another way, the first major curve 204 repositions the distal tip 211 so that it is not at the distal most location of the select catheter 200. In some examples, the tip 211 may face a generally proximal direction as opposed to when the select catheter 200 is in a generally linear constrained configuration (e.g., the select catheter 200 is forced to take on a straight shape).

Referring to FIG. 40, the first major curve 204 may also curve away from the reference plane 200F. FIG. 40 illustrates the reference plane 200F which is rotated at about 90 degrees relative to the view of the reference plane 200F in FIG. 39. In one example, the first major curve 204 forms an angle 200E with the reference plane 200F (e.g., also with the proximal second section 208) within an inclusive range of about 5 degrees to 25 degrees. In some examples, the angle 200E may include the following: about 5, 6, 7, 8, 9, 10, 11, 12, 13, 14, 15, 16, 17, 18, 19, 20, 21, 22, 23, 24, or 25 degrees. In some examples, the angle 200E may include the following: about 11, 11.01, 11.02, 11.03, 11.04, 11.05, 11.06, 11.07, 11.08, 11.09, 11.1, 11.11, 11.12, 11.13, 11.14, 11.15, 11.16, 11.17, 11.18, 11.19, 11.2, 11.21, 11.22, 11.23, 11.24, 11.25, 11.26, 11.27, 11.28, 11.29, 11.3, 11.31, 11.32, 11.33, 11.34, 11.35, 11.36, 11.37, 11.38, 11.39, 11.4, 11.41, 11.42, 11.43, 11.44, 11.45, 11.46, 11.47, 11.48, 11.49, 11.5, 11.51, 11.52, 11.53, 11.54, 11.55, 11.56, 11.57, 11.58, 11.59, 11.6, 11.61, 11.62, 11.63, 11.64, 11.65, 11.66, 11.67, 11.68, 11.69, 11.7, 11.71, 11.72, 11.73, 11.74, 11.75, 11.76, 11.77, 11.78, 11.79, 11.8, 11.81, 11.82, 11.83, 11.84, 11.85, 11.86, 11.87, 11.88, 11.89, 11.9, 11.91, 11.92, 11.93, 11.94, 11.95, 11.96, 11.97, 11.98, 11.99, or 12 degrees.

In this respect, the distal first section 210, as well as its distal tip 211, is positioned proximally of the first major curve 204, but is further angled such that the distal first section 210 is non-parallel to the proximal second section 208. In other words, the distal tip 211 of the distal first section 210 is positioned at a larger distance away from the proximal second section 208 than portions of the distal first section 210 at or near the first major curve 204.

FIGS. 41-43 illustrate other views of the distal region 200A of the select catheter 200 that better illustrate the curvature of the first major curve 204. FIG. 41 illustrates a view from a distal vantage point of the distal region 200A when unconstrained such that the first major curve 204 is closest to the viewer and the proximal second section 208 extends away from the viewer. In FIG. 42, the proximal second section 208 generally extends towards the viewer and the first major curve 204 is positioned further away from the viewer. FIG. 43 illustrates another side perspective view of the distal region 200A with the proximal second section 208 being closest to the viewer and the first major curve 204 being furthest from the viewer.

The distal region 200A of the select catheter 200 may also include one or more minor curves. In one example, a minor curve is defined as a curve that is less than the first major curve 204 or within an inclusive range of about 90 to 190 degrees away from the proximal second section 208.

Returning to the example of FIGS. 39 and 41, a minor curve 206 may be imparted to a portion of the distal first section 210, creating an angle 200G between segment 200C and segment 200N. Angle 200G may be any of the minor curve angles of this specification. In some examples, the angle of the minor curve 206 may be within an inclusive range of about 90 to about 190 degrees, and mores specifically the following: about 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, or 190 degrees between segment 200C and segment 200N.

In one example, the minor curve 206 may remain substantially within a first plane (plane not shown) such that the entire distal first section 210 (length 200J) may be substantially located in the single plane, which is the case in the example of FIGS. 39 and 40, or the minor curve 206 may further curve out of a first plane (not shown) in which a proximal portion of the distal first section 210 is substantially located, similar to the description of the first major curve 204.

Depending on the length of the distal first section 210 and the curvature of the minor curve 206, the tip 211 of the distal first section 210 may be located at different distances from the proximal second section 208. When viewing from the perspective of FIG. 39, length 200S (e.g., the distance from between the tip 211 and the proximal second section 208) may be within an inclusive range of about 1.3 cm to about 6.3 cm. More specific examples may include the following: about 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.8, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, or 6.3 cm. When viewing from the perspective of FIG. 40, distance 200D (e.g., another component of the distance from between the tip 211 and the proximal second section 208) may be within an inclusive range of about 0.4 cm to 2.0 cm. More specific examples may include the following: about 0.4, 0.5, 0.6, 0.7, 0.8, 0.9, 1.0, 1.1, 1.2, 1.3, 1.4, 1.5, 1.6, 1.7, 1.8, 1.9, or 2.0 cm. More specific examples may include the following: about 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, or 1.2 cm.

As further illustrated in FIGS. 39 and 40, in one example, the length 200J between the distal tip 211 of the distal first section 210 and an apex of the curved end of the first major curve 204 may be within an inclusive range of about 3-8 cm. More specific examples may include the following: about 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9 7.0, 7.1, 7.2, 7.3, 7.4, 7.5, 7.6, 7.7, 7.8, 7.9 or 8.0 cm. A distance 200S between the distal tip 211 and the proximal second section 208 when viewing from position of FIG. 39 may be within an inclusive range of about 1 to 2 cm. More specific examples may include the following: about 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, or 2 cm.

In one example, the length 200H of the proximal second section 208 may be within an inclusive range of about .5 cm to 15 cm. More specific examples may include the following: about .5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, or 15.0 cm. The proximal second section 208 may be substantially straight or may have a relatively shallow curve along part or all of its length. The length 200K of the straight portion (i.e., without the first major curve 204 may be within an inclusive range of about .5 cm to 15 cm, similar to the specific examples for length 200H. In one specific example, the length 200H is about 6.35 cm and length 200K is about 5.8 cm.

In one example, when unconstrained, the select catheter may have a length of about 140 cm (a range of about 135-145 cm) between a distal tip 211 and the start of a strain relief sleeve connected to a catheter hub 207.

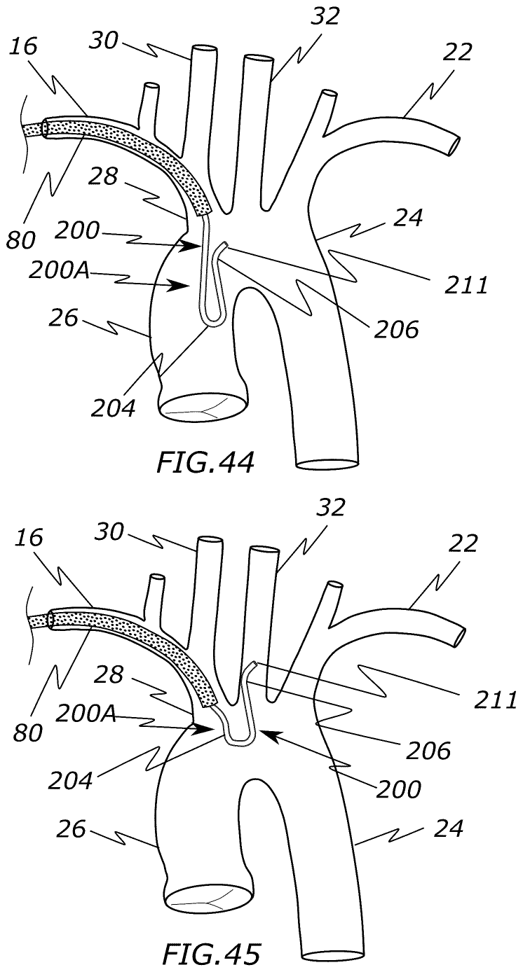

FIGS. 44 and 45 illustrate various views of a method of using the select catheter 200 of FIGS. 39-43. A guide catheter 80 is advanced via a right radial approach (as previously described) such that its distal end is located in or near the brachiocephalic artery 28 (alternatively, in the right subclavian artery 16 or the aortic arch 24). Next, as seen in FIG. 44, the distal portion of the select catheter 200 is advanced out of the guide catheter 80 and into the aortic arch 24 such that it takes on its unconstrained, expanded, memorized shape. Once the memorized shape has been achieved, a physician may proximally retract the select catheter 200 such that some or all of the proximal second section 208 is back within the guide catheter 80. Since the distal region is sized and shaped similar to the distance and location of the left common carotid artery 32, the proximal movement moves the distal tip 211 into the left common carotid artery 32 as illustrated in FIG. 45.

Depending on the physician's use, the select catheter 200 may then be further distally advanced to move the distal portion of the select catheter 200 further up the left common carotid artery 32 and the guide catheter 80 may be advanced over the select catheter 200 into the left common carotid artery 32. Alternatively or additionally, a guidewire may be advanced through the select catheter 200 into the left common carotid artery 32. Variations and combinations thereof may alternatively be performed. Depending on the plan of a physician, the select catheter 200 may be removed and additional working catheters (or guidewires) may be used for an intended treatment.

As previously discussed, the select catheter 200 may have a stiffness profile that provides certain stiffnesses at certain distances from its distal tip that helps prevent loss of access of the select catheter when tracking a guidewire through it or larger outer catheter 80 over it. By providing certain stiffness changes at certain locations along the length of the select catheter 200, the select catheter may better maintain access to a desired vessel during a procedure.

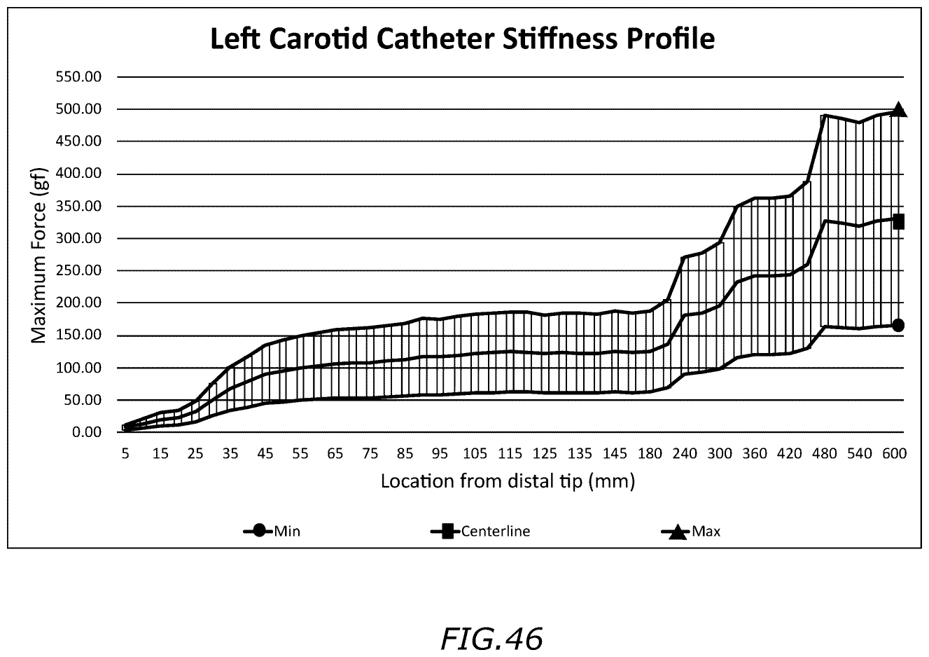

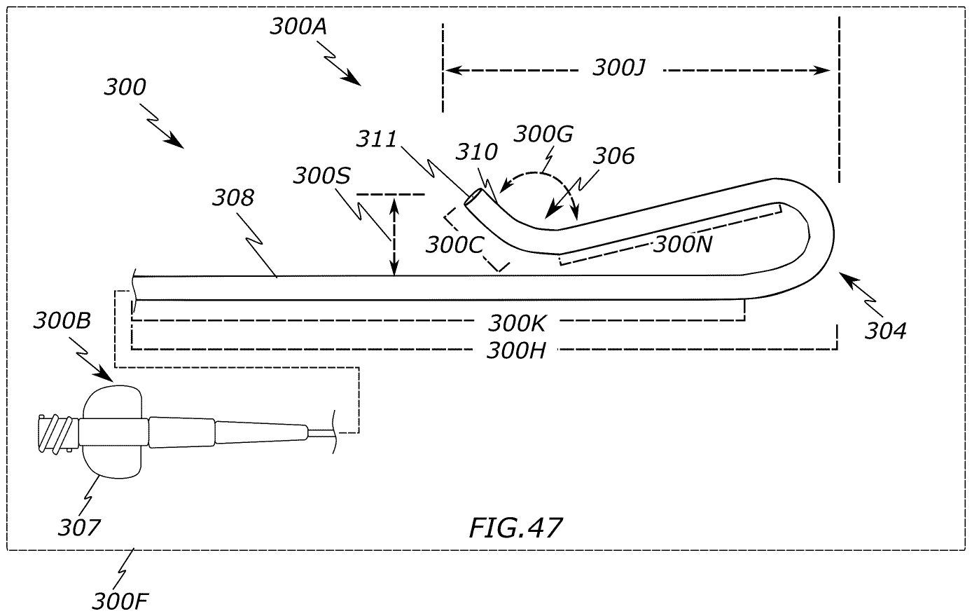

Table 1 below and FIG. 46 illustrate an example stiffness profile for a select catheter 200. An average stiffness is provided at different lengths from a distal tip 211 of the select catheter 200, but also minimum and maximum stiffnesses are also provided. Hence, these minimum and maximum values may be thought of as a range of stiffness at any specified location in the table or figure.

In this example data, it can be seen that the stiffness only moderately increases between about 0-25 mm, greatly increases between about 25-45 mm, and then generally remains relatively constant or slightly increases between about 45-145 mm. This stiffness profile may particularly benefit the previously described dimensions and curvature of the select catheter 200 for accessing the left common carotid artery 32 via the right subclavian artery 16, as previously described.

This stiffness profile, in combination with the three-dimensional catheter shape, allows the select catheter to “auto-form” or otherwise achieve the secondary shape/configuration (U-shape) either without contacting a vessel wall (or aortic valve) or with minimal force applied to the vessel wall (or aortic valve). This may avoid or minimize any damage to the vessel wall and the dislodgement of plaque. In addition, it may also provide the physician or operator with easier, faster, and more consistent access to the right/left common carotid arteries, as previously described. In situations where “time is brain” such as a stroke, this increased speed and consistency may also improve patient outcomes by allowing the physician or operator to remove an obstruction faster and more reliably. It may also facilitate access to challenging anatomy (e.g., Type III aortic arch) and/or anatomy that cannot be accessed with prior art catheters.

The stiffness profile may be generated using standardized test methods such as ASTM D790-17 or ISO 178. The data in Tables 1-3 were generated using similar test methods combining both a two-point bend test for the catheter length that is 0-20 mm from the distal tip (distal-most 20 mm) and a three-point bend test for distances further than 20 mm from the distal tip. The three-point bend test was performed with a 2 cm gap defining the area of test, where the 2 cm gap is centered on the catheter distance being tested (e.g., for the measurement at 40 mm from distal tip, the gap spans the length of the catheter between 30-50 mm from the distal tip). During both two-point and three-point bend testing, a load sensor (“anvil”) measured the force required to displace the catheter in the measurement area by 1.5 mm, with the measurements being taken in grams force or “g/f. The stiffness may be determined with the known formula of force/displacement distance. Depending on the exact protocol of the testing, the force values and curvature may be higher or lower than the values presented herein, but may achieve a similar stiffness curve or profile that is “normalized” or would look very similar if a “scalar” were applied. Hence, various other two-point bend and three-point bend testing methods (both standardized and proprietary) may produce “normalized” measurement values in grams force (g/f), but scaled proportionally (e.g., measurements using other test methods with larger displacement may produce larger grams force measurements (e.g., 10%-100% higher), while measurements using a test method with smaller displacement may produce smaller grams force measurements (e.g., 10%-75% lower); other testing parameters such as gap size, temperature, humidity, etc. may also affect the measurement values, but the overall stiffness profile should scale proportionally to remain normalized.

TABLE 1

| Location | Average | Lower | Higher |
|---|---|---|---|
| from the | Maximum | Maximum | Maximum |
| tip (mm) | Force (gf) | Force (gf) | Force (gf) |
| 5.00 | 8.00 | 4.00 | 12.00 |
| 10.00 | 13.50 | 6.75 | 20.25 |
| 15.00 | 20.00 | 10.00 | 30.00 |
| 20.00 | 22.58 | 11.29 | 33.86 |
| 25.00 | 32.24 | 16.12 | 48.36 |
| 30.00 | 50.65 | 25.33 | 75.98 |
| 35.00 | 66.95 | 33.47 | 100.42 |
| 40.00 | 77.96 | 38.98 | 116.95 |
| 45.00 | 89.55 | 44.77 | 134.32 |
| 50.00 | 94.87 | 47.44 | 142.31 |
| 55.00 | 99.08 | 49.54 | 148.61 |
| 60.00 | 103.18 | 51.59 | 154.76 |
| 65.00 | 105.78 | 52.89 | 158.67 |
| 70.00 | 107.47 | 53.74 | 161.21 |
| 75.00 | 107.63 | 53.82 | 161.45 |
| 80.00 | 110.34 | 55.17 | 165.51 |
| 85.00 | 111.86 | 55.93 | 167.80 |
| 90.00 | 117.32 | 58.66 | 175.98 |
| 95.00 | 117.02 | 58.51 | 175.53 |
| 100.00 | 119.46 | 59.73 | 179.19 |
| 105.00 | 121.88 | 60.94 | 182.83 |
| 110.00 | 123.13 | 61.57 | 184.70 |
| 115.00 | 124.56 | 62.28 | 186.85 |
| 120.00 | 123.99 | 61.99 | 185.98 |
| 125.00 | 121.24 | 60.62 | 181.85 |
| 130.00 | 123.17 | 61.59 | 184.76 |
| 135.00 | 122.62 | 61.31 | 183.93 |
| 140.00 | 121.69 | 60.84 | 182.53 |
| 145.00 | 125.31 | 62.65 | 187.96 |
| 150.00 | 123.03 | 61.52 | 184.55 |
| 180.00 | 124.68 | 62.34 | 187.02 |
| 210.00 | 136.76 | 68.38 | 205.14 |
| 240.00 | 181.04 | 90.52 | 271.56 |
| 270.00 | 184.85 | 92.43 | 277.28 |
| 300.00 | 195.09 | 97.55 | 292.64 |
| 330.00 | 232.51 | 116.26 | 348.77 |
| 360.00 | 241.95 | 120.98 | 362.93 |
| 390.00 | 241.92 | 120.96 | 362.88 |
| 420.00 | 243.29 | 121.64 | 364.93 |
| 450.00 | 259.08 | 129.54 | 388.62 |
| 480.00 | 326.62 | 163.31 | 489.94 |
| 510.00 | 323.47 | 161.73 | 485.20 |
| 540.00 | 319.45 | 159.72 | 479.17 |
| 570.00 | 327.31 | 163.65 | 490.96 |
| 600.00 | 330.66 | 165.33 | 495.99 |

In one example, the select catheter 200 may have a stiffness at about 110 mm from the distal tip 211 within an inclusive range of about 68.38 gf and 205.14 gf. In another example, the select catheter 200 may have a stiffness at about 80 mm from a distal tip 211 of the select catheter within an inclusive range of about 55.17 gf and 165.51 gf. In another example, the select catheter 200 may have a stiffness at about 20 mm from the distal tip 211 within an inclusive range of about 11.29 gf and 33.86 gf. In another example, the select catheter 200 may have a stiffness at about 10 mm from the distal tip 211 within an inclusive range of about 6.75 gf and 20.25 gf. In another example, the select catheter 200 may have a stiffness at about 5 mm from the distal tip 211 within an inclusive range of about 4 gf and 12 gf. In another example, the select catheter 200 may have a stiffness within all of the previously disclosed ranges.

In another example, the select catheter 200 may have an average stiffness at about 110 mm from the distal tip 211 of about 136.76 gf, about 80 mm from the distal tip 211 of about 110.34 gf, at about 20 mm from the distal tip 211 of about 22.58 gf, at about 10 mm from the distal tip 211 of about 13.5 gf, and at about 5 mm from the distal tip 211 of about 8 gf.

As previously discussed, the three-dimensional unconstrained shape and dimensions, as well as the stiffness profile may all be included in the same example catheter, or they may be used in any combination together, including being used alone.

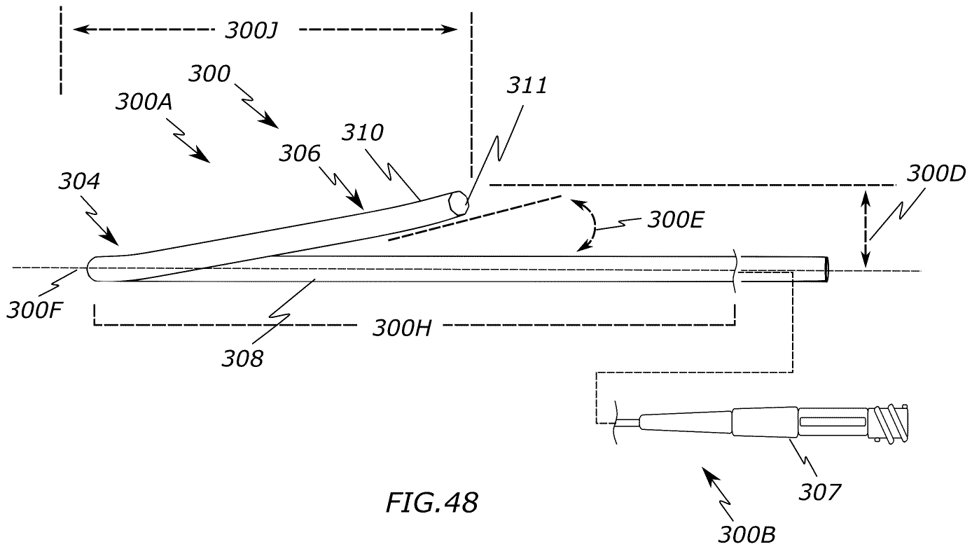

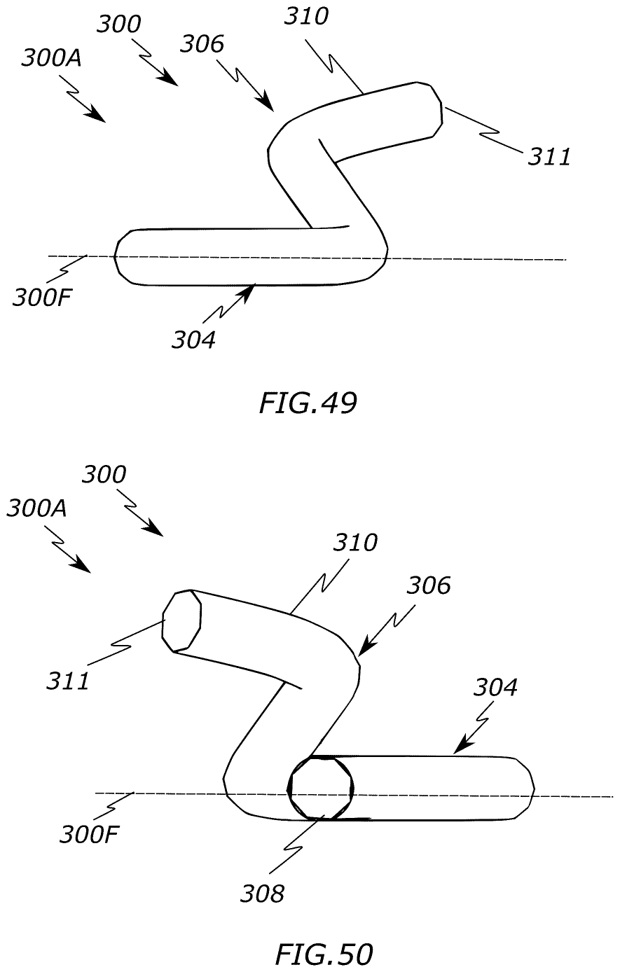

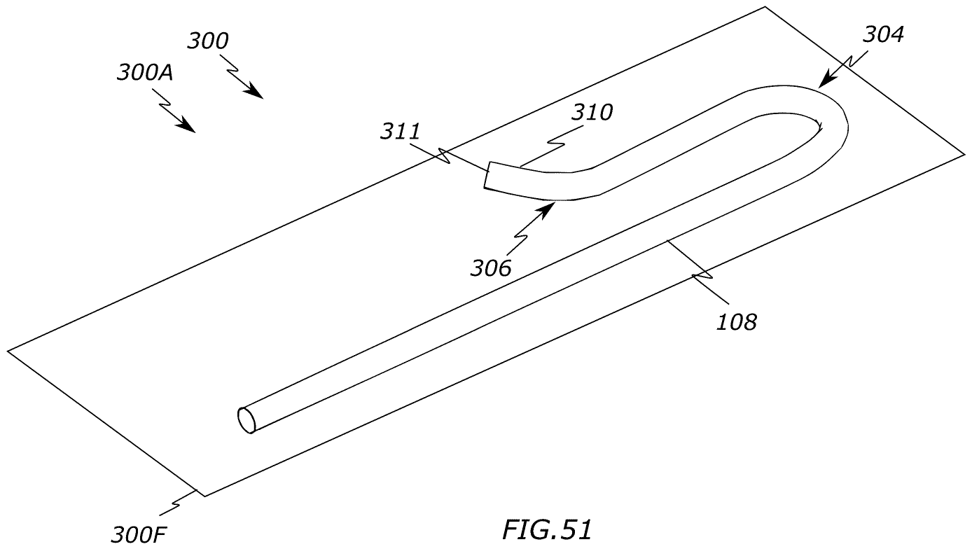

FIGS. 47-51 illustrate an example of a select catheter 300 with a curved distal region. FIG. 47 illustrates a side view of the select catheter 300 in a first rotational position relative to a plane 300F. FIG. 48 illustrates a side view of the select catheter 300 in a second rotational position that is about perpendicular to the position shown in the view of FIG. 47. FIG. 49 illustrates an end view of the select catheter 300 from distal end perspective. FIG. 50 illustrates an end view of the select catheter 300 from a proximal end perspective. FIG. 51 illustrates a perspective view of the select catheter 300. The reference plane 300F is included in each of these figures to help convey the shape of the select catheter 300. In FIG. 47, the face of the reference plane 300F faces the viewer and in FIG. 48 the edge of the reference plane 300F faces the viewer.

In one example, the select catheter 300 may comprise a generally elongated shape or body that may have a lumen therethrough (e.g., a guidewire lumen) or may not have a lumen. In some examples, the select catheter 300 may include a variety of different structural materials that impart different flexibilities, such as inner wire coils, inner braided wires, and outer polymer jackets of different durometer.

Returning to the example of FIG. 47, the select catheter 300 may include a distal region 300A and a proximal region 300B. The proximal region 300B may include a proximal catheter hub 307 or a similar terminal end that allows connection to other medical devices and/or access to the interior lumen, if present. The distal region 300A may extend a distance from the proximal region 300B and may terminate at a distal tip 311 of the select catheter 300. As described further below, the distal region 300A includes an imparted secondary shape that forms when at least part of the distal region 300A is unconstrained (e.g., not constrained by a larger outer catheter). In one example, the distal region may have a length within an inclusive range of about 3 cm and about 30 cm when straightened.

In one example, this distal region 300A may include a distal first section 310 (also denoted by length 300J) and a proximal second section 308 (also denoted by length 300H). The distal first section 310 and the proximal second section 308 may be unitary with each other where the distal first section 310 is distal and connected to the proximal second section 308 when the select catheter 300 is in a constrained linear shape. Adjacent ends of the distal first section 310 and the proximal second section 308 form a first major curve 304 with each other. In one example, a major curve may be defined as either the largest curve when the distal region 300A is unconstrained or a curve that bends beyond 45 degrees.

As seen in FIGS. 47 and 48, the first major curve 304 is formed with adjacent ends of the distal first section 310 and the proximal second section 308 and which does not remain substantially in a single plane 300F. In one example, as can be seen in FIG. 47, the first major curve 304 may curve back in a generally proximal direction of about 192 degrees when viewed against the face of the reference plane 300F or a radius of curvature of about. 102 inch. However, a variety of different angles may also be possible, such as an inclusive range of 160 degrees to 220 degrees. In more specific examples, the following may be possible: 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, or 220 degrees. Example radius of curvature include an inclusive range of 0.08 inch to 0.22 inch, or more specifically: 0.08, 0.09, 0.1, 0.11, 0.12, 0.13, 0.14, 0.15, 0.16, 0.17, 0.18, 0.19, 0.2, 0.21, or 0.22 inch. Hence, the tip 311 of the select catheter 300 is positioned generally proximally of the first major curve 304 when the distal region 300A is unconstrained. Put another way, the first major curve 304 repositions the distal tip 311 so that it is not at the distal most location of the select catheter 300. In some examples, the tip 311 may face a generally proximal direction as opposed to when the select catheter 300 is in a generally linear constrained configuration (e.g., the select catheter 300 is forced to take on a straight shape).

Referring to FIG. 48, the first major curve 304 may also curve away from the reference plane 300F. FIG. 48 illustrates the reference plane 300F which is rotated at about 90 degrees relative to the view of the reference plane 300F in FIG. 47. In one example, the first major curve 304 forms an angle 300E with the reference plane 300F (e.g., also with the proximal second section 308) within an inclusive range of about 5 degrees to 25 degrees. In some examples, the angle 300E may include the following: about 5, 6, 7, 8, 9, 10, 11, 12, 13, 14, 15, 16, 17, 18, 19, 20, 21, 22, 23, 24, or 25 degrees. In some examples, the angle 300E may include the following: about 10.0, 10.05, 10.1, 10.15, 10.2, 10.25, 10.3, 10.35, 10.4, 10.45, 10.5, 10.55, 10.6, 10.65, 10.7, 10.75, 10.8, 10.85, 10.9, 10.95, or 11.0 degrees.

In this respect, the distal first section 310, as well as its distal tip 311, is positioned proximally of the first major curve 304, but is further angled such that the distal first section 310 is non-parallel to the proximal second section 308. In other words, the distal tip 311 of the distal first section 310 is positioned at a larger distance away from the proximal second section 308 than portions of the distal first section 310 at or near the first major curve 304.

FIGS. 49-51 illustrate other views of the distal region 300A of the select catheter 300 that better illustrate the curvature of the first major curve 304. FIG. 49 illustrates a view from a distal vantage point of the distal region 300A when unconstrained such that the first major curve 304 is closest to the viewer and the proximal second section 308 extends away from the viewer. In FIG. 50, the proximal second section 308 generally extends towards the viewer and the first major curve 304 is positioned further away from the viewer. FIG. 51 illustrates another side perspective view of the distal region 300A with the proximal second section 308 being closest to the viewer and the first major curve 304 being furthest from the viewer.

The distal region 300A of the select catheter 300 may also include one or more minor curves. In one example, a minor curve is defined as a curve that is less than the first major curve 304 or within an inclusive range of about 90 to 190 degrees away from the proximal second section 308.

Returning to the example of FIGS. 47 and 48, a minor curve 306 may be imparted to a portion of the distal first section 310, creating an angle 300G between segment 300C and segment 300N. Angle 300G may be within an inclusive range of about 90 to about 190 or any of the previously disclosed minor curve angles. In some examples, the angle of the minor curve 306 may be the following: about 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, or 190 degrees between segment 300C and segment 300N. In some examples, the angle of the minor curve 306 may be the following: about 120, 120.01, 120.02, 120.03, 120.04, 120.05, 120.06, 120.07, 120.08, 120.09, 120.1, 120.11, 120.12, 120.13, 120.14, 120.15, 120.16, 120.17, 120.18, 120.19, 120.2, 120.21, 120.22, 120.23, 120.24, 120.25, 120.26, 120.27, 120.28, 120.29, 120.3, 120.31, 120.32, 120.33, 120.34, 120.35, 120.36, 120.37, 120.38, 120.39, 120.4, 120.41, 120.42, 120.43, 120.44, 120.45, 120.46, 120.47, 120.48, 120.49, 120.5, 120.51, 120.52, 120.53, 120.54, 120.55, 120.56, 120.57, 120.58, 120.59, 120.6, 120.61, 120.62, 120.63, 120.64, 120.65, 120.66, 120.67, 120.68, 120.69, 120.7, 120.71, 120.72, 120.73, 120.74, 120.75, 120.76, 120.77, 120.78, 120.79, 120.8, 120.81, 120.82, 120.83, 120.84, 120.85, 120.86, 120.87, 120.88, 120.89, 120.9, 120.91, 120.92, 120.93, 120.94, 120.95, 120.96, 120.97, 120.98, 120.99, or 121.00.

In one example, the minor curve 306 may remain substantially within a first plane (plane not shown) such that the entire distal first section 310 (length 300J) may be substantially located in the single plane, which is the case in the example of FIGS. 47 and 48, or the minor curve 306 may further curve out of a first plane (not shown) in which a proximal portion of the distal first section 310 is substantially located, similar to the description of the first major curve 304.

Depending on the length of the distal first section 310 and the curvature of the minor curve 306, the tip 311 of the distal first section 310 may be located at different distances from the proximal second section 308. When viewing from the perspective of FIG. 47, length 300S (e.g., the distance from between the tip 311 and the proximal second section 308) may be within an inclusive range of about .6 cm to about 4 cm. More specific examples may include the following: about 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, 2, 2.01, 2.02, 2.03, 2.04, 2.05, 2.06, 2.07, 2.08, 2.09, 2.1, 2.11, 2.12, 2.13, 2.14, 2.15, 2.16, 2.17, 2.18, 2.19, 2.2, 2.21, 2.22, 2.23, 2.24, 2.25, 2.26, 2.27, 2.28, 2.29, 2.3, 2.31, 2.32, 2.33, 2.34, 2.35, 2.36, 2.37, 2.38, 2.39, 2.4, 2.41, 2.42, 2.43, 2.44, 2.45, 2.46, 2.47, 2.48, 2.49, 2.5, 2.51, 2.52, 2.53, 2.54, 2.55, 2.56, 2.57, 2.58, 2.59, 2.6, 2.61, 2.62, 2.63, 2.64, 2.65, 2.66, 2.67, 2.68, 2.69, 2.7, 2.71, 2.72, 2.73, 2.74, 2.75, 2.76, 2.77, 2.78, 2.79, 2.8, 2.81, 2.82, 2.83, 2.84, 2.85, 2.86, 2.87, 2.88, 2.89, 2.9, 2.91, 2.92, 2.93, 2.94, 2.95, 2.96, 2.97, 2.98, 2.99, 3, 3.01, 3.02, 3.03, 3.04, 3.05, 3.06, 3.07, 3.08, 3.09, 3.1, 3.11, 3.12, 3.13, 3.14, 3.15, 3.16, 3.17, 3.18, 3.19, 3.2, 3.21, 3.22, 3.23, 3.24, 3.25, 3.26, 3.27, 3.28, 3.29, 3.3, 3.31, 3.32, 3.33, 3.34, 3.35, 3.36, 3.37, 3.38, 3.39, 3.4, 3.41, 3.42, 3.43, 3.44, 3.45, 3.46, 3.47, 3.48, 3.49, 3.5, 3.51, 3.52, 3.53, 3.54, 3.55, 3.56, 3.57, 3.58, 3.59, 3.6, 3.61, 3.62, 3.63, 3.64, 3.65, 3.66, 3.67, 3.68, 3.69, 3.7, 3.71, 3.72, 3.73, 3.74, 3.75, 3.76, 3.77, 3.78, 3.79, 3.8, 3.81, 3.82, 3.83, 3.84, 3.85, 3.86, 3.87, 3.88, 3.89, 3.9, 3.91, 3.92, 3.93, 3.94, 3.95, 3.96, 3.97, 3.98, 3.99, or 4 cm.

When viewing from the perspective of FIG. 48, distance 300D (e.g., another component of the distance from between the tip 311 and the proximal second section 308) may be within an inclusive range of about 0.1 cm to 1.0 cm. More specific examples may include the following: about 0.1, 0.101, 0.102, 0.103, 0.104, 0.105, 0.106, 0.107, 0.108, 0.109, 0.11, 0.111, 0.112, 0.113, 0.114, 0.115, 0.116, 0.117, 0.118, 0.119, 0.12, 0.121, 0.122, 0.123, 0.124, 0.125, 0.126, 0.127, 0.128, 0.129, 0.13, 0.131, 0.132, 0.133, 0.134, 0.135, 0.136, 0.137, 0.138, 0.139, 0.14, 0.141, 0.142, 0.143, 0.144, 0.145, 0.146, 0.147, 0.148, 0.149, 0.15, 0.151, 0.152, 0.153, 0.154, 0.155, 0.156, 0.157, 0.158, 0.159, 0.16, 0.161, 0.162, 0.163, 0.164, 0.165, 0.166, 0.167, 0.168, 0.169, 0.17, 0.171, 0.172, 0.173, 0.174, 0.175, 0.176, 0.177, 0.178, 0.179, 0.18, 0.181, 0.182, 0.183, 0.184, 0.185, 0.186, 0.187, 0.188, 0.189, 0.19, 0.191, 0.192, 0.193, 0.194, 0.195, 0.196, 0.197, 0.198, 0.199, or 0.2 cm.

As further illustrated in FIGS. 47 and 48, in one example, the length 300J between the distal tip 311 of the distal first section 310 and an apex of the curved end of the first major curve 304 may be within an inclusive range of about 2-5 cm. More specific examples may include the following: about 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5 cm. A length 300S between the distal tip 311 and the proximal second section 308 when viewing from position of FIG. 47 may be within an inclusive range of about .2 cm to .5 cm. More specific examples may include the following: about 2.3, 2.4, 2.4, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, or 6.3 cm.

In one example, the length 300H of the proximal second section 308 may be within an inclusive range of about .5 cm to 15 cm. More specific examples may include the following: about .5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, or 15.0 cm. More specific examples may include the following: about 5.5, 5.6, 5.7, 5.8, 5.9, 6, 6.1, 6.2, 6.3, 6.4, or 6.5 cm. The proximal second section 308 may be substantially straight or may have a relatively shallow curve along part or all of its length. The length 300K of the straight portion (i.e., without the first major curve 304 may be within an inclusive range of about .5 cm to 15 cm, similar to the specific examples for length 300H. In one specific example, the length 300H is about 5.8 cm and length 300K is about 5.0 cm.

In one example, when unconstrained, the select catheter may have a length of about 140 cm (a range of about 135-145 cm) between a distal tip 311 and the start of a strain relief sleeve connected to a catheter hub 307.

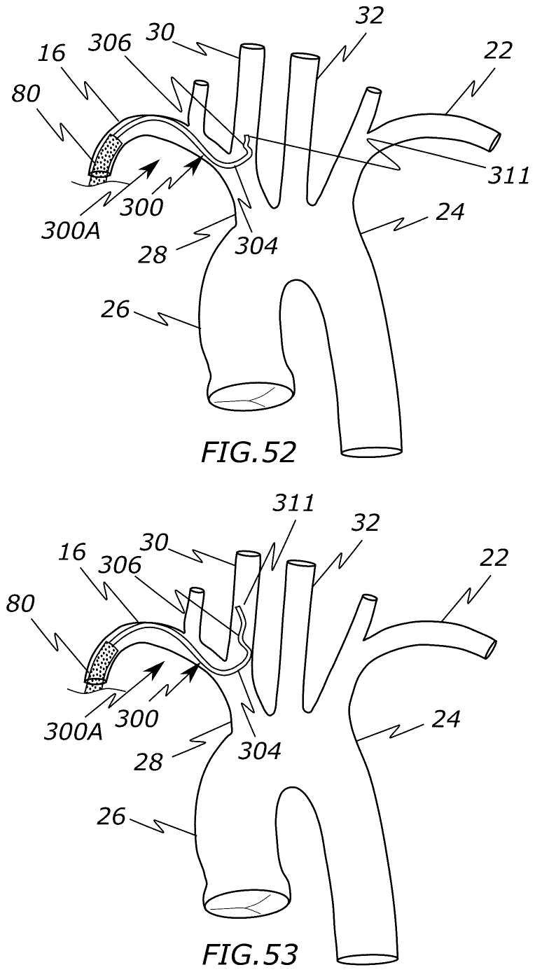

FIGS. 52 and 53 illustrate various views of a method of using the select catheter 300 of FIGS. 47-51. A guide catheter 80 is advanced via a right radial approach (as previously described) such that its distal end is located in or near the brachiocephalic artery 28 (alternatively, in the right subclavian artery 16 or the aortic arch 24). Next, as seen in FIG. 52, the distal portion of the select catheter 300 is advanced out of the guide catheter 80 and into the aortic arch 24 such that it takes on its unconstrained, expanded, memorized shape. Once the memorized shape has been achieved, a physician may proximally retract the select catheter 300 such that some or all of the proximal second section 308 is back within the guide catheter 80. Since the distal region is sized and shaped similar to the distance and location of the right common carotid artery 30, the proximal movement moves the distal tip 311 into the right common carotid artery 30 as illustrated in FIGS. 52 and 53.

Depending on the physician's use, the select catheter 300 may then be further distally advanced to move the distal portion of the select catheter 300 further up the right common carotid artery 30 and the guide catheter 80 may be advanced over the select catheter 200 into the right common carotid artery 30. Alternatively or additionally, a guidewire may be advanced through the select catheter 300 into the right common carotid artery 30. Variations and combinations thereof may alternatively be performed. Depending on the plan of a physician, the select catheter 300 may be removed and additional working catheters (or guidewires) may be used for an intended treatment.

As previously discussed, the select catheter 300 may have a stiffness profile that provides certain stiffnesses at certain distances from its distal tip that helps prevent loss of access of the select catheter when tracking a guidewire through it or larger outer catheter 80 over it. By providing certain stiffness changes at certain locations along the length of the select catheter 300, the select catheter may better maintain access to a desired vessel during a procedure.

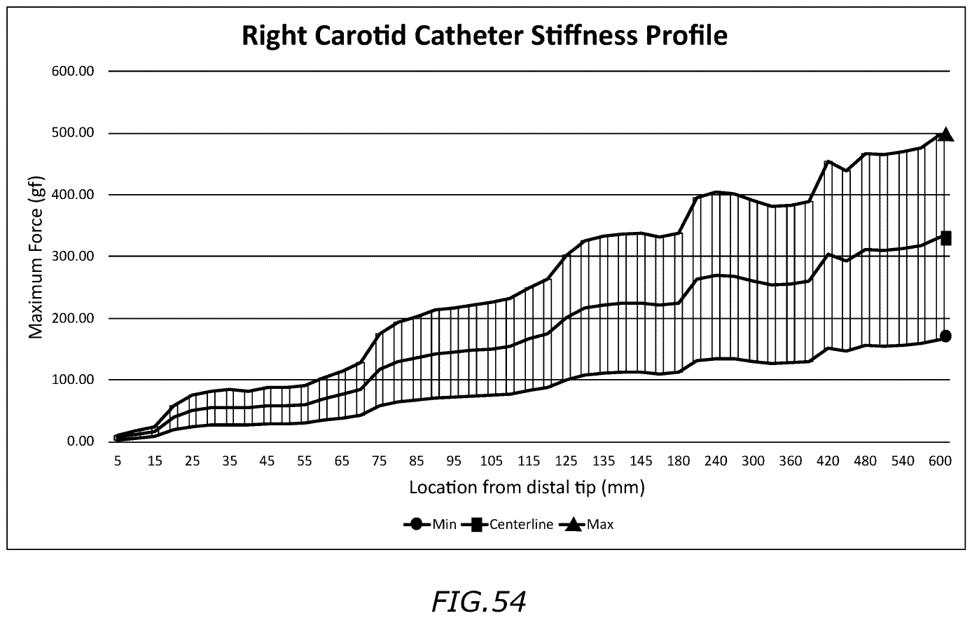

Table 2 below and FIG. 54 illustrate an example stiffness profile for a select catheter 300. An average stiffness is provided at different lengths from a distal tip 311 of the select catheter 300, but also minimum and maximum stiffnesses are also provided. Hence, these minimum and maximum values may be thought of as a range of stiffness at any specified location in the table or figure.

In this example data, it can be seen that the stiffness only moderately increases between about 0-15 mm, greatly increases between about 15-20 mm, only moderately increases or remains near constant between about 20-70 mm, and greatly increases between about 70-75 mm. This stiffness profile may particularly benefit the previously described dimensions and curvature of the select catheter 300 for accessing the right common carotid artery 30 via the right subclavian artery 16, as previously described.

This stiffness profile, in combination with the three-dimensional catheter shape, allows the select catheter to “auto-form” or otherwise achieve the secondary shape/configuration (U-shape) either without contacting a vessel wall (or aortic valve) or with minimal force applied to the vessel wall (or aortic valve). This may avoid or minimize any damage to the vessel wall and the dislodgement of plaque. In addition, it may also provide the physician or operator with easier, faster, and more consistent access to the right/left common carotid arteries, as previously described. In situations where “time is brain” such as a stroke, this increased speed and consistency may also improve patient outcomes by allowing the physician or operator to remove an obstruction faster and more reliably. It may also facilitate access to challenging anatomy (e.g., Type III aortic arch) and/or anatomy that cannot be accessed with prior art catheters.

TABLE 2

| Location | Average | Lower | Higher |
|---|---|---|---|
| from the | Maximum | Maximum | Maximum |
| tip (mm) | Force (gf) | Force (gf) | Force (gf) |
| 5 | 6.40 | 3.20 | 9.60 |
| 10 | 12.08 | 6.04 | 18.11 |
| 15 | 16.20 | 8.10 | 24.30 |
| 20 | 38.96 | 19.48 | 58.44 |
| 25 | 49.91 | 24.95 | 74.86 |
| 30 | 54.60 | 27.30 | 81.90 |
| 35 | 55.98 | 27.99 | 83.97 |
| 40 | 54.93 | 27.46 | 82.39 |
| 45 | 58.05 | 29.02 | 87.07 |
| 50 | 58.07 | 29.04 | 87.11 |
| 55 | 60.15 | 30.07 | 90.22 |
| 60 | 68.80 | 34.40 | 103.20 |
| 65 | 76.36 | 38.18 | 114.53 |
| 70 | 85.10 | 42.55 | 127.65 |
| 75 | 116.72 | 58.36 | 175.07 |
| 80 | 128.99 | 64.50 | 193.49 |
| 85 | 135.59 | 67.79 | 203.38 |
| 90 | 142.72 | 71.36 | 214.07 |
| 95 | 144.91 | 72.45 | 217.36 |
| 100 | 147.97 | 73.98 | 221.95 |
| 105 | 150.32 | 75.16 | 225.48 |
| 110 | 154.85 | 77.42 | 232.27 |
| 115 | 166.36 | 83.18 | 249.54 |
| 120 | 175.42 | 87.71 | 263.12 |
| 125 | 201.36 | 100.68 | 302.04 |
| 130 | 216.50 | 108.25 | 324.75 |
| 135 | 222.06 | 111.03 | 333.09 |
| 140 | 224.32 | 112.16 | 336.48 |
| 145 | 224.82 | 112.41 | 337.23 |
| 150 | 220.57 | 110.28 | 330.85 |
| 180 | 225.09 | 112.54 | 337.63 |
| 210 | 263.72 | 131.86 | 395.58 |
| 240 | 269.50 | 134.75 | 404.25 |
| 270 | 267.71 | 133.85 | 401.56 |
| 300 | 260.26 | 130.13 | 390.39 |
| 330 | 254.54 | 127.27 | 381.81 |
| 360 | 255.24 | 127.62 | 382.86 |
| 390 | 259.42 | 129.71 | 389.13 |
| 420 | 303.17 | 151.58 | 454.75 |
| 450 | 292.69 | 146.34 | 439.03 |
| 480 | 311.31 | 155.66 | 466.97 |
| 510 | 309.62 | 154.81 | 464.43 |
| 540 | 313.06 | 156.53 | 469.59 |
| 570 | 316.99 | 158.49 | 475.48 |
| 600 | 331.67 | 165.84 | 497.51 |

In one example, the select catheter 300 may have a stiffness at about 110 mm from the distal tip 311 within an inclusive range of about 77.42 gf and 232.27 gf. In another example, the select catheter 300 may have a stiffness from the distal tip 311 at about 80 mm from a distal tip 311 of the select catheter within an inclusive range of about 64.50 gf and 193.49 gf. In another example, the select catheter 300 may have a stiffness from the distal tip 311 at about 20 mm within an inclusive range of about 19.48 gf and 58.44 gf. In another example, the select catheter 300 may have a stiffness from the distal tip 311 at about 10 mm within an inclusive range of about 6.04 gf and 18.11 gf. In another example, the select catheter 300 may have a stiffness from the distal tip 311 at about 5 mm within an inclusive range of about 3.20 gf and 9.60 gf. In another example, the select catheter 300 may have a stiffness within all of the previously disclosed ranges.

In another example, the select catheter 300 may have an average stiffness at about 110 mm from the distal tip 311 of about 154.85 gf, about 80 mm from the distal tip 311 of about 128.99 gf, at about 20 mm from the distal tip 311 of about 38.96 gf, at about 10 mm from the distal tip 311 of about 12.08 gf, and at about 5 mm from the distal tip 311 of about 6.40 gf.

As previously discussed, the three-dimensional unconstrained shape and dimensions, as well as the stiffness profile may all be included in the same example catheter, or they may be used in any combination together, including being used alone.

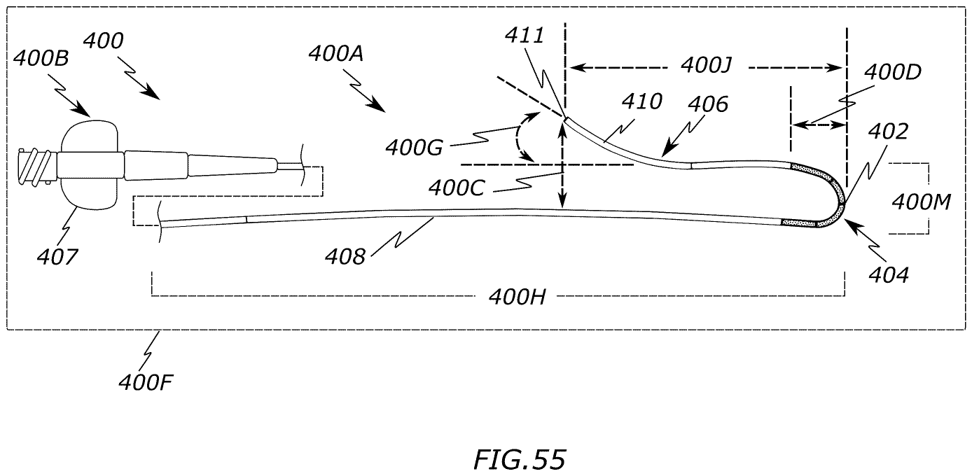

FIGS. 55-63 illustrates an example of a select catheter 400 intended for use via a femoral approach. This select catheter 400 includes similarities to some of the previously discussed select catheters but may have different dimensions, curvatures, and the inclusion of a select portion of length having a lubricious coating. This select catheter 400 will be discussed generally first, and then in greater detail with regard to the figures.

With regard to the low-friction or lubricious coating, it that may be positioned on a portion of a distal region of an inner catheter next to areas of a relatively higher friction of the distal region. While a select catheter may particularly benefit from such a low-friction coating or low-friction area, any catheter that includes a pre-shaped or unconstrained curvature that may be deployed through a larger catheter or sheath may also benefit. Hence, this low-friction aspect should not be limited to only select catheters. Additionally, any of the catheters discussed in this specification may include this low-friction coating.

In one example of a select catheter, the coating may be located along only a curved region (e.g., a major curve) and not along the very distal length of the inner catheter. In another example, the coating may be located along only the curved region and some adjacent portions of the curve (e.g., within an inclusive range of about 1 mm to about 10 mm). This may create a first region of relatively increased friction between the distal end of the inner catheter and a short distance thereafter, followed by a second region of relatively decreased friction proximally after the first region and extending over the curved region, followed by a third region of relatively increased friction proximally after the second region. Since select catheters tend to have a pre-shaped curve (e.g., a major curve) when unconstrained, when they are constrained or straightened within a larger catheter (e.g., guide catheter or balloon catheter), the curved region tends to provide increased force, and therefore friction, on the inner lumen of the larger catheter. In other words, the curved region tends to press against the walls of the inner lumen of the larger catheter while also forcing regions that are immediately adjacent to the curved region against an opposite side of the wall of the inner lumen of the larger catheter. Hence, the low-friction or lubricious coating may help reduce friction in these areas.

If the low-friction or lubricious coating was continued to the very distal end of the select catheter and along much of the proximal portion of the select catheter, it may tend to be relatively slippery when located within a vessel off of the aortic arch 24, and therefore when the larger catheter or a guidewire is advanced over/through the select catheter, the select catheter may tend to slip out of the vessel it is positioned in. Therefore, the higher friction region or regions on one or both sides of the curved region of the select catheter may help maintain the select catheter in a desired vessel it has been positioned in (e.g., the brachiocephalic artery 28, the right common carotid artery 30, or into the left common carotid artery 32) by providing increased friction against the patient's vessels. Hence, the combination of lower and higher friction regions may help reduce friction when advancing the select catheter within a larger catheter and may provide good stability (e.g., remains in place) within a vessel from the aortic arch 24.

As previously discussed, existing select catheters only curve within or remain substantially within a single plane which can make accessing some vessels in an aortic arch difficult, particularly for Type II and Type III anatomies as previously discussed with regard to FIG. 2B. By including curvature along the distal tip of the select catheter so that at least some of a distal region of the select catheter does not remain within a single plane (e.g., a major curve that positions the free distal tip of the catheter in a generally proximal direction while positioning a distal most region in a non-parallel orientation to proximal adjacent regions of the select catheter), different vessels can be more easily accessed, particularly for Type II and Type III anatomies. For example, the brachiocephalic artery 28 and the left common carotid artery 32 can be more easily accessed.

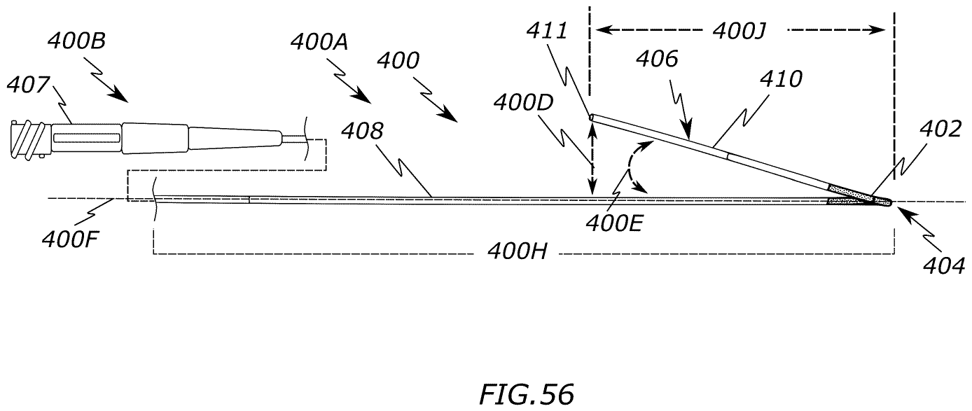

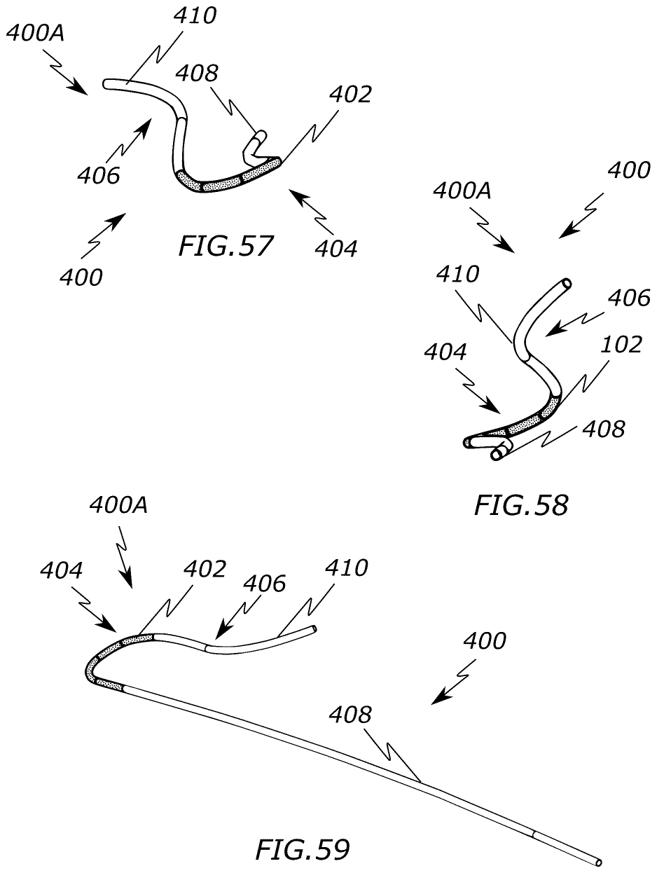

FIGS. 55-59 illustrate an example of a select catheter 400 with a curved distal region. FIG. 55 illustrates a side view of the select catheter 400 in a first rotational position relative to a plane 400F. FIG. 56 illustrates a side view of the select catheter 400 in a second rotational position that is about perpendicular to the position shown in the view of FIG. 55. FIG. 57 illustrates an end view of the select catheter 400 from distal end perspective. FIG. 58 illustrates an end view of the select catheter 400 from a proximal end perspective. FIG. 59 illustrates a perspective view of the select catheter 400. The reference plane 400F is included in both FIGS. 55 and 56 to help convey the shape of the select catheter 400. In FIG. 55, the face of the reference plane 400F faces the viewer and in FIG. 4 the edge of the reference plane 400F faces the viewer.

In one example, the select catheter 400 may comprise a generally elongated shape or body that may have a lumen therethrough (e.g., a guidewire lumen) or may not have a lumen. In some examples, the select catheter 400 may include a variety of different structural materials that impart different flexibilities, such as inner wire coils, inner braided wires, and outer polymer jackets of different durometer.

Returning to the example of FIG. 55, the select catheter 400 may include a distal region 400A and a proximal region 400B. The proximal region 400B may include a proximal catheter hub 407 or a similar terminal end that allows connection to other medical devices and/or access to the interior lumen, if present. The distal region 400A may extend a distance from the proximal region 400B and may terminate at a distal tip 411 of the select catheter 400. As described further below, the distal region 400A includes an imparted shape that forms when at least part of the distal region 400A is unconstrained (e.g., not constrained by a larger outer catheter). In one example, the distal region may have a length between about 3 cm and about 30 cm when straightened.

In one example, this distal region 400A may include a distal first section 410 and a proximal second section 408. The distal first section 410 and the proximal second section 408 may be unitary with each other where the distal first section 410 is distal and connected to the proximal second section 408 when the select catheter 400 is in a constrained linear shape. Adjacent ends of the distal first section 410 and the proximal second section 408 form a major curve with each other. In one example, a major curve may be defined as either the largest curve when the distal region 400A is unconstrained or a curve that bends beyond 45 degrees.

As seen in FIGS. 55 and 56, the first major curve 404 is formed with adjacent ends of the distal first section 410 and the proximal second section 408 and which does not remain substantially in a single plane. In one example, as can be seen in FIG. 55, the first major curve 404 may curve back in a generally proximal direction of about 480 degrees when viewed against the face of the reference plane 400F. However, a variety of different angles may also be possible, such as an inclusive range of 460 degrees to 200 degrees. In more specific examples, the following may be possible: 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 degrees. Hence, the tip 411 of the select catheter 400 is positioned generally proximally of the first major curve 404 when the distal region 400A is unconstrained. Put another way, the first major curve 404 repositions the distal tip 411 so that it is not at the distal most location of the select catheter 400. In some examples, the tip 411 may face a generally proximal direction as opposed to when the select catheter 400 is in a generally linear constrained configuration (e.g., the select catheter 400 is forced to take on a straight shape). In one example, an outer diameter 400M at the first major curve 404 is about. 084 inch.

Referring to FIG. 56, the first major curve 404 may also curve away from the reference plane 400F. FIG. 56 illustrates the reference plane 400F which is rotated at about 90 degrees relative to the view of the reference plane 400F in FIG. 55. In one example, the first major curve 404 forms an angle 400E with the reference plane 400F (e.g., also with the proximal second section 408) within an inclusive range of about 5 degrees to 25 degrees. In some examples, the angle 400E may include the following: about 5, 6, 7, 8, 9, 10, 11, 12, 13, 14, 15, 16, 17, 18, 19, 20, 21, 22, 23, 24, or 25 degrees.

In this respect, the distal first section 410, as well as its distal tip 411, is positioned proximally of the first major curve 404, but is further angled such that the distal first section 410 is non-parallel to the proximal second section 408. In other words, the distal tip 411 of the distal first section 410 is positioned at a larger distance away from the proximal second section 408 than portions of the distal first section 410 at or near the first major curve 404.

FIGS. 57-59 illustrate other views of the distal region 400A of the select catheter 400 that better illustrate the curvature of the first major curve 404. FIG. 57 illustrates a view from a distal vantage point of the distal region 400A when unconstrained such that the first major curve 404 is closest to the viewer and the proximal second section 408 extends away from the viewer. In FIG. 58, the proximal second section 408 generally extends towards the viewer and the first major curve 404 is positioned further away from the viewer. FIG. 59 illustrates another side perspective view of the distal region 400A with the proximal second section 408 being closest to the viewer and the first major curve 404 being furthest from the viewer.

The distal region 400A of the select catheter 400 may also include one or more minor curves. In one example, a minor curve is defined as a curve that is less than the first major curve 404 or within an inclusive range of about 1 to 45 degrees relative to the reference plane 400F or a proximal portion of the distal region 400A. In some examples, the angles of the minor curve may also include the following: about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 degrees relative to the reference plane 400F or a proximal portion of the distal region 400A.

Returning to the example of FIGS. 55 and 56, a minor curve 406 may be imparted to a portion of the distal first section 410, creating an angle 400G relative to the reference plane 400F or a proximal portion of the distal first section 410. Angle 400G may be any of the previously disclosed minor curve angles.

In one example, the minor curve 406 may remain substantially within a first plane (plane not shown) such that the entire distal first section 410 may be substantially located in the single plane, which is the case in the example of FIGS. 55 and 56, or the minor curve 406 may further curve out of a first plane (not shown) in which a proximal portion of the distal first section 410 is substantially located, similar to the description of the first major curve 404.

Depending on the length of the distal first section 410 and the curvature of the minor curve 406, the tip 411 of the distal first section 410 may be located at different distances from the proximal second section 408. When viewing from the perspective of FIG. 55, distance 400C (e.g., one component of the distance from between the tip 411 and the proximal second section 408) may be within an inclusive range of about 1.3 cm to about 6.3 cm. More specific examples may include the following: about 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.8, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, or 6.3 cm. When viewing from the perspective of FIG. 56, distance 400D (e.g., another component of the distance from between the tip 411 and the proximal second section 408) may be within an inclusive range of about 0.4 cm to 2.0 cm. More specific examples may include the following: about 0.4, 0.5, 0.6, 0.7, 0.8, 0.9, 1.0, 1.1, 1.2, 1.3, 1.4, 1.5, 1.6, 1.7, 1.8, 1.9, or 2.0 cm.

As further illustrated in FIGS. 55 and 56, in one example, the length 400J between the distal tip 411 of the distal first section 410 and an apex of the curved end of the first major curve 404 may be within an inclusive range of about 5-8 cm. More specific examples may include the following: about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9 7.0, 7.1, 7.2, 7.3, 7.4, 7.5, 7.6, 7.7, 7.8, 7.9 or 8.0 cm. A distance 400C between the distal tip 411 and the proximal second section 408 when viewing from position of FIG. 55 may be within an inclusive range of about 2.3-6.3 cm. More specific examples may include the following: about 2.3, 2.4, 2.4, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3 cm.

In one example, the length 400H of the proximal second section 408 may be within an inclusive range of about .5 cm to 15 cm. More specific examples may include the following: about .5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, or 15.0 cm. The proximal second section 408 may be substantially straight or may have a relatively shallow curve along part or all of its length.

In one example, when unconstrained, the select catheter may have a length of about 150 cm (+/−3) between a distal tip 411 and the start of a strain relief sleeve connected to a catheter hub 407.

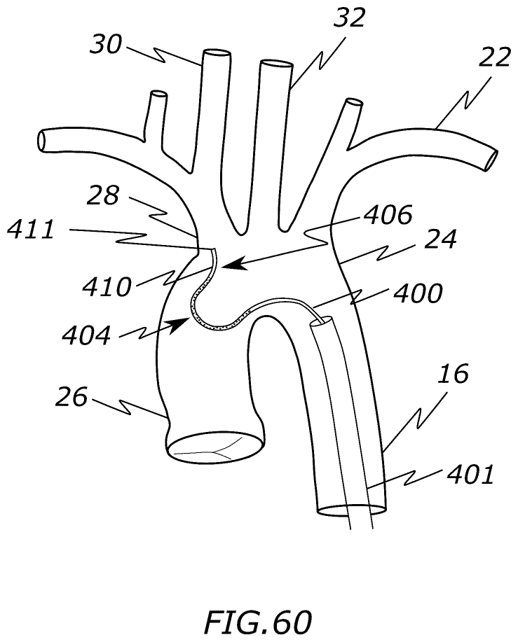

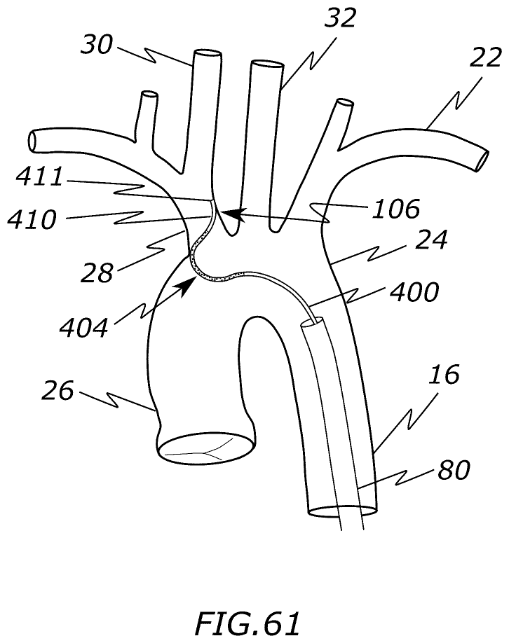

FIGS. 60 and 61 illustrate one example method of use of an example catheter with the previously described unconstrained shape. Referring first to FIG. 60, a larger outer catheter 80 may be advanced near or into an aortic arch 24 of a patient. Next, the select catheter 400 may be advanced out of the outer catheter 80. As seen in FIG. 60, the three-dimensional shape of the first major curve 404 helps angle the distal tip 411 towards a desired vessel opening, such as the brachiocephalic artery 28 and the right common carotid artery 30, the left common carotid artery 32, or left subclavian artery 22. The minor curve 406 may also further assist in placement of the distal tip 411. In FIG. 61, the select catheter 400 is further advanced into the brachiocephalic artery 28 and toward the right common carotid artery 30. At this point, either the outer catheter 80 may be advanced over the select catheter 400 and into the brachiocephalic artery 28 and the right common carotid artery 30, or a guidewire (not shown) may be advanced through the select catheter 400 and into the brachiocephalic artery 28 and the right common carotid artery 30. The outer catheter 80 and/or guidewire may be further advanced to a desired location and a treatment may be performed on the patient.

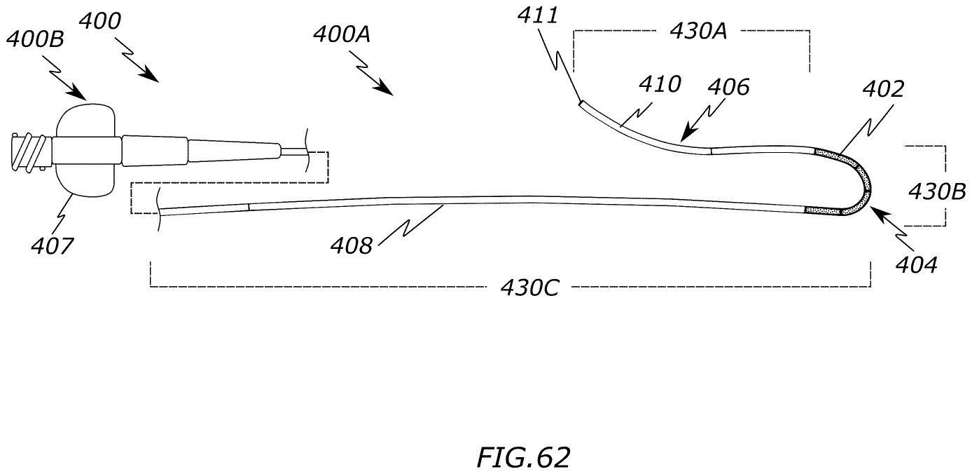

FIG. 62 illustrates a side view of the select catheter 400. As previously discussed, in one example, the select catheter 400 may include a low-friction or lubricious coating 402 that is positioned on only a portion of a distal region 400A of a select catheter 400. In some examples, the areas without the lubricious coating may have a relatively higher coefficient of friction relative to the areas with the lubricious coating. For example, the lubricious coating 402 may be located along only part or all of the first major curve 404 and not along the very distal length of the distal first section 410 or along a proximal length of the proximal second section 408. Alternatively, the lubricious coating 402 may extend a short distance distally and/or proximally beyond the first major curve 404 (e.g., about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 cm). Optionally, a separate area of the lubricious coating 402 may be included on any additional curves, such as the minor curve 406, in a similar manner described for the major curve 404.

As seen in FIG. 62, these example arrangements of the lubricious coating 402 may include the following example. First, a distal first portion 430A comprises a relatively increased friction between the distal tip 411 of the select catheter 400 and a short distance thereafter. That distal first portion 430A is followed by a second portion 430B of lubricious coating 402 of relatively decreased friction proximally after the first portion 430A. The second portion 430B may be positioned over some of, all of, or beyond the first major curve 404. The second portion 430B may be followed by a proximal third portion 430C of relatively increased friction relative to the lubricious coating 402. Further, in one example, a separate section of the lubricious coating 402 may extend along part or all of the minor curve 406 (not shown). In one example, portion may mean a section or region extending along at least some of a length of the select catheter 400.

While the example described in FIG. 62 may comprise a lubricious coating 402 to create at least one region with a relatively decreased coefficient of friction, other techniques may also be used to create similar patterns of coefficients of friction. For example, the outer polymer jacket of the select catheter 400 may comprise tubular portions that may exhibit different coefficients of friction.

While the example described in FIG. 62 may comprise a lubricious coating 402 that completely encompasses the outer surface or circumference of the select catheter 400, other examples may comprise a lubricous coating 402 (or the equivalent jacket material) along only a portion of the outer surface or circumference of the select catheter 400. In one example, the lubricous coating 402 (or the equivalent jacket material) may be located along an outer surface of a curved portion and the inner surface of a curved portion may be free of the lubricious coating 402 or may otherwise have a higher coefficient of friction.

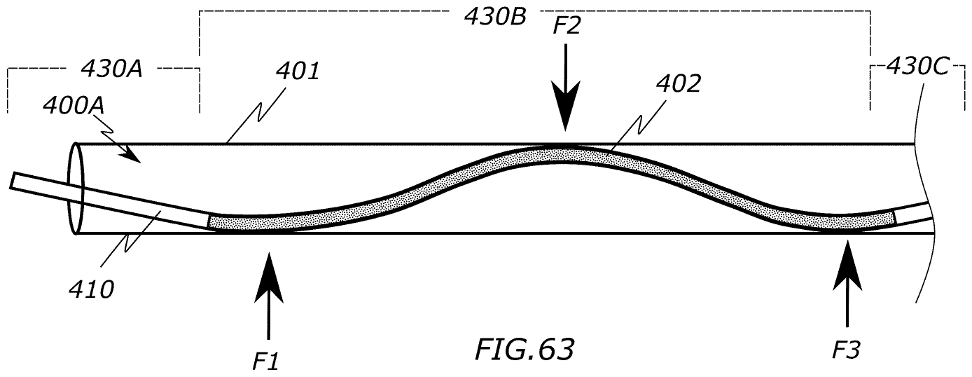

FIG. 63 illustrates a side view of the select catheter 400 within a larger catheter 80. In one example, since the select catheter 400 may have a pre-shaped curved shape when unconstrained, particularly the first major curve 404 when it is constrained or straightened within a larger catheter 80 (e.g., guide catheter or balloon catheter), portions where the first major curve 404 contact the inner lumen of the larger catheter 80 may create relatively higher levels of friction, and therefore relatively higher levels of force that tends to resist movement, on the inner lumen of the larger catheter 80. The areas with enlarged arrows (e.g., F1, F2, F3) of FIG. 63 illustrate example areas of this relatively higher friction. In some examples, the relatively higher frictional force may be the same or different at the areas F1, F2, and/or F3 and may depend on the degree of curvature of the select catheter 400. Hence, the second portion 430B of the lubricious coating 402 may cover at least the area of the middle arrow (e.g., F2) but may also cover the areas of all three arrows, thereby reducing the friction and force needed to advance the select catheter 400 through the larger catheter 80.

In some examples, at least the distal first portion 430A of the select catheter 400 with relatively higher friction (e.g., non-coated portion) may help maintain the select catheter 400 in a desired vessel it has initially been positioned in (e.g., the brachiocephalic artery 28, the right common carotid artery 30, or into the left common carotid artery 32). The proximal third portion 430C may provide further friction with portions of the patient's vessels and therefore further support a position of the select catheter 400. Hence, the combination of lower and higher friction sections may help reduce friction when advancing the select catheter 400 within a larger catheter 80 and may provide good stability (e.g., remains in place) within a vessel from the aortic arch 24 when the select catheter 400 is partially advanced out of the larger catheter 80.

In one example, the distal first portion 430A is within an inclusive range of about 4.8 cm to 6.0 cm or in a specific example of about 5.4 cm. The second portion 430B may have a length within an inclusive range of about 1 cm to 5 cm in a specific example of about 3.0 cm when the select catheter 400 is measured straight.

In another example, the distal first portion 430A is within an inclusive range of about 3.8 to 5.0 cm or in a specific example of about 4.4 cm. The second portion 430B may have a length within an inclusive range of about 1 cm to 5 cm in a specific example of about 3.0 cm when the select catheter 400 is measured straight.

The distal first portion 430A may exhibit between about 5-15 times less friction than the second portion 430B having the low friction or lubricious coating 402, and in a more specific example about 10 times. For example, the distal first portion 430A may have a frictional value of within an inclusive range of about 761 gf to 363 gf, and the second portion 430B may have a frictional value of about 29 gf. The proximal third portion 430C may exhibit between 5-15 times less friction than the second portion 430B having the low friction or lubricious coating 402, and in a more specific example about 10 times, as well as a frictional value of within an inclusive range of about 761 gf or 363 gf. The frictional values of the distal first portion 430A and proximal third portion 430C may be the same or different. The These frictional gf values may be measured by clamping a specific area and applying a clamping force of about 1 lbs. (+/−. 2 lbs.) of force, pulling the sample through the clamp, and recording the frictional force values.

Generally, the lubricious coating 402 may comprise a variety of different low-friction, lubricious, and/or hydrophilic coatings. One or more such coatings are discussed in PCT/US2023/067044 filed May 16, 2023, which is incorporated by reference in its entirety. The distal first portion 430A and other portions of the select catheter 400 may comprise a variety of thermoplastic polymers such as, poly(amides), poly(ethylene terephthalate), poly(urethanes), poly(ether sulfones), poly(carbonates), poly(vinyl chloride), copolymers thereof, and derivatives thereof, such as Peebax.

Disclosed coatings (e.g., lubricious coating 402 or any coatings on remaining portions of the select catheter 400) can comprise, for example, multiple coats, such as, for example, a base coat and a top coat. The base coat may function as a “tie” layer between a thermoplastic polymer of the select catheter 400 and the top coat. The base coat may be designed to adhere to the catheter and provide binding sites for the attachment of the top coat. The top coat may be designed to adhere to the base coat and provide lubricity to reduce the frictional forces created when the catheter is moved in the vasculature.

The base coat may comprise a polymer that is a copolymer of a first tetrahydrofurfuryl acrylate monomer and at least one other monomer with functional groups capable of further chemical reaction such as hydroxyl, amine, and carboxylic acid groups. The at least one other monomer including hydroxyl groups can be hydroxyethyl methacrylate, hydroxyethyl acrylate, hydroxypropyl acrylate, hydroxypropyl methacrylate, hydroxybutyl acrylate, hydroxybutyl methacrylate, combinations thereof, and derivatives thereof. The at least one other monomer including amine groups can be N-(3-aminopropyl) methacrylamide, 2-aminoethyl methacrylate, 2-aminoethyl methacrylamide, combinations thereof, and derivatives thereof. The at least one other monomer including carboxylic acids can be acrylic acid, methacrylic acid, beta-carboxyethyl acrylate, combinations thereof, and derivatives thereof.

The top coat polymer may comprise a core, hydrophilic polymer that is derivatized with polymerizable groups. The core hydrophilic polymer may be any naturally occurring or synthetic polymer, derivatives thereof and combinations thereof. In some embodiments, the core hydrophilic polymer is at least to some degree, soluble in water.

The structure of the core hydrophilic polymer can be linear or branched, including graft, star, comb, brush, and dendrimer structures.

Polymers used for the top coat may comprise but are not limited to naturally occurring polymers such as proteins, collagen, albumin, fibrin, elastin, polypeptides, oligonucleotides, polysaccharides, hyaluronic acid, gelatin, chitosan, alginate, cellulose, carboxymethyl cellulose, hydroxymethyl cellulose, hydroxypropyl cellulose, and dextran.

Polymers used for the top coat can comprise, but are not limited to synthetic polymers such as poly(ethers), poly(ethylene glycol), poly(ethylene oxide), poly(propylene glycol), poly(lactams), poly(vinylpyrrolidone), poly(acrylates), poly(urethanes), poly(anhydrides), poly(amino acids), poly(carboxylic acids), poly(amides), poly(vinyl alcohol), and poly(phosphazenes).

Molecular weights of the hydrophilic polymers can range from, for example, about 500 amu to about 100,000 amu or from about 1,000 amu to about 40,000 amu.

Reactive groups, such as, but not limited to acrylates and/or methacrylates, can be added to the polymer via any convenient reactive moiety, such as hydroxyls, amines, or carboxylic acids, with a derivatization compound. In some embodiments, the derivatization compound can be a hetero-bifunctional compound. One moiety can react with the hydroxyl, amine, and/or carboxylic acid groups of the copolymer. The other moiety can be an acrylate or methacrylate group.

The derivatization compound may comprise acryloyl chloride, methacryloyl chloride, 2-isocyanatoethyl acrylate, 2-isocyanatoethyl methacrylate, acrylic acid N-hydroxysuccinimide ester, methacrylic acid Nhydroxysuccinimide ester, hetero-bifunctional poly(ethylene glycol) with acrylate and isocyanate groups, combinations thereof, and derivatives thereof.

While the lubricious coating 402 is described as located over portions of the select catheter 400 (e.g., the first major curve 404), any catheter and any unconstrained curve, bend, or angle may include such a coating over some of, the entirety of, or even beyond the catheter curve. Note that while curve is used below, it should be considered synonymous with the term bend.

An example curve may have an angle of at least 10 degrees, at least 15 degrees, at least 20 degrees, at least 25 degrees, at least 30 degrees, at least 35 degrees, at least 40 degrees, at least 45 degrees, at least 50 degrees, at least 55 degrees, at least 60 degrees, at least 65 degrees, at least 70 degrees, at least 75 degrees, at least 80 degrees, at least 85 degrees, at least 90 degrees, at least 95 degrees, at least 100 degrees, at least 105 degrees, at least 110 degrees, at least 115 degrees, at least 120 degrees, at least 125 degrees, at least 130 degrees, at least 135 degrees, at least 140 degrees, at least 145 degrees, at least 150 degrees, at least 155 degrees, at least 160 degrees, at least 165 degrees, at least 170 degrees, at least 175 degrees, at least 180 degrees, at least 185 degrees, at least 190 degrees, at least 195 degrees, at least 200 degrees, at least 205 degrees, at least 210 degrees, at least 220 degrees, at least 225 degrees, at least 230 degrees, at least 235 degrees, at least 240 degrees, at least 245 degrees, at least 250 degrees, at least 255 degrees, at least 260 degrees, at least 265 degrees, at least 270 degrees, at least 275 degrees, at least 280 degrees, at least 285 degrees, at least 290 degrees, at least 295 degrees, at least 300 degrees, at least 305 degrees, at least 310 degrees, at least 315 degrees, at least 320 degrees, at least 325 degrees, at least 330 degrees, at least 335 degrees, at least 340 degrees, at least 345 degrees, at least 350 degrees, at least 355 degrees, or the like.

An example curve may have an angle of at most 10 degrees, at most 15 degrees, at most 20 degrees, at most 25 degrees, at most 30 degrees, at most 35 degrees, at most 40 degrees, at most 45 degrees, at most 50 degrees, at most 55 degrees, at most 60 degrees, at most 65 degrees, at most 70 degrees, at most 75 degrees, at most 80 degrees, at most 85 degrees, at most 90 degrees, at most 95 degrees, at most 100 degrees, at most 105 degrees, at most 110 degrees, at most 115 degrees, at most 120 degrees, at most 125 degrees, at most 130 degrees, at most 135 degrees, at most 140 degrees, at most 145 degrees, at most 150 degrees, at most 155 degrees, at most 160 degrees, at most 165 degrees, at most 170 degrees, at most 175 degrees, at most 180 degrees, at most 185 degrees, at most 190 degrees, at most 195 degrees, at most 200 degrees, at most 205 degrees, at most 210 degrees, at most 220 degrees, at most 225 degrees, at most 230 degrees, at most 235 degrees, at most 240 degrees, at most 245 degrees, at most 250 degrees, at most 255 degrees, at most 260 degrees, at most 265 degrees, at most 270 degrees, at most 275 degrees, at most 280 degrees, at most 285 degrees, at most 290 degrees, at most 295 degrees, at most 300 degrees, at most 305 degrees, at most 310 degrees, at most 315 degrees, at most 320 degrees, at most 325 degrees, at most 330 degrees, at most 335 degrees, at most 340 degrees, at most 345 degrees, at most 350 degrees, at most 355 degrees, or the like.

An example curve may have an angle of 10 degrees, 15 degrees, 20 degrees, 25 degrees, 30 degrees, 35 degrees, 40 degrees, 45 degrees, 50 degrees, 55 degrees, 60 degrees, 65 degrees, 70 degrees, 75 degrees, 80 degrees, 85 degrees, 90 degrees, 95 degrees, 100 degrees, 105 degrees, 110 degrees, 115 degrees, 120 degrees, 125 degrees, 130 degrees, 135 degrees, 140 degrees, 145 degrees, 150 degrees, 155 degrees, 160 degrees, 165 degrees, 170 degrees, 175 degrees, 180 degrees, 185 degrees, 190 degrees, 195 degrees, 200 degrees, 205 degrees, 210 degrees, 220 degrees, 225 degrees, 230 degrees, 235 degrees, 240 degrees, 245 degrees, 250 degrees, 255 degrees, 260 degrees, 265 degrees, 270 degrees, 275 degrees, 280 degrees, 285 degrees, 290 degrees, 295 degrees, 300 degrees, 305 degrees, 310 degrees, 315 degrees, 320 degrees, 325 degrees, 330 degrees, 335 degrees, 340 degrees, 345 degrees, 350 degrees, 355 degrees, or the like.

An example curve may have an angle of between about 10 and 350 degrees, between about 20 and 340 degrees, between about 30 and 330 degrees, between about 40 and 320 degrees, between about 50 and 310 degrees, between about 60 and 300 degrees, between about 70 and 290 degrees, between about 80 and 280 degrees, between about 90 and 270 degrees, between about 100 and 260 degrees, between about 110 and 250 degrees, between about 120 and 240 degrees, between about 130 and 230 degrees, between about 140 and 220 degrees, between about 150 and 210 degrees, between about 160 and 200 degrees, between about 170 and 190 degrees, or the like.

An example curve may have an angle of between about 10 and 40 degrees, between about 20 and 50 degrees, between about 30 and 60 degrees, between about 40 and 70 degrees, between about 50 and 80 degrees, between about 60 and 90 degrees, between about 70 and 100 degrees, between about 80 and 110 degrees, between about 90 and 120 degrees, between about 100 and 130 degrees, between about 110 and 140 degrees, between about 120 and 150 degrees, between about 130 and 160 degrees, between about 140 and 170 degrees, between about 150 and 180 degrees, between about 160 and 190 degrees, between about 170 and 200 degrees, between about 180 and 210 degrees, between about 190 and 220 degrees, between about 200 and 230 degrees, between about 210 and 240 degrees, between about 220 and 250 degrees, between about 230 and 260 degrees, between about 240 and 270 degrees, between about 250 and 280 degrees, between about 260 and 290 degrees, between about 270 and 300 degrees, between about 280 and 310 degrees, between about 290 and 320 degrees, between about 300 and 330 degrees, between about 310 and 340 degrees, between about 320 and 350 degrees, or the like.

As previously discussed, the select catheter 400 may have a stiffness profile that provides certain stiffnesses at certain distances from its distal tip that helps prevent loss of access of the select catheter when tracking a guidewire through it or larger outer catheter 80 over it. For example, some existing select catheters include a relatively large jump in stiffness at about 8 cm from their distal tips, which can result in the aforementioned loss of access. By providing a less abrupt transition, the select catheter may better maintain access to a desired vessel during a procedure.

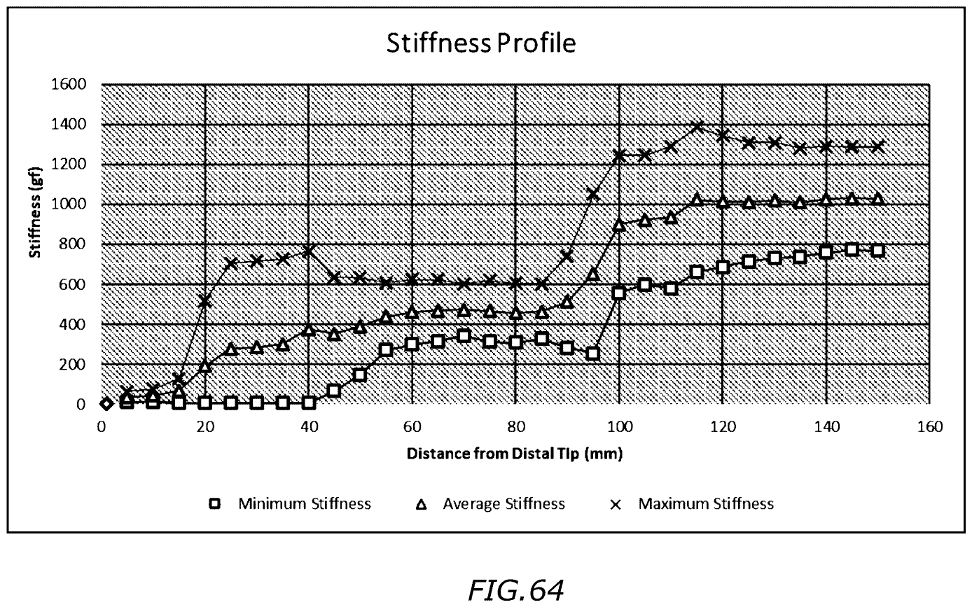

Table 3 below and FIG. 64 illustrate an example stiffness profile for a select catheter 400. An average stiffness is provided at different lengths from a distal tip 411 of the select catheter 400, but also minimum and maximum stiffnesses are also provided. Hence, these minimum and maximum values may be thought of as a range of stiffness at any specified location in the table or figure.

TABLE 3

| Location | Minimum | Average | Maximum |
|---|---|---|---|
| from distal | Stiffness | Stiffness | Stiffness |
| tip (mm) | (gf) | (gf) | (gf) |
| 5 | 11.7 | 37.3 | 62.9 |
| 10 | 12.4 | 44.2 | 76.1 |
| 15 | 7.0 | 68.5 | 129.9 |
| 20 | 7.0 | 192.9 | 520.5 |
| 25 | 7.0 | 277.2 | 706.1 |
| 30 | 7.0 | 284.9 | 716.8 |
| 35 | 7.0 | 300.5 | 725.9 |
| 40 | 7.0 | 376.5 | 766.3 |
| 45 | 66.7 | 351.3 | 635.8 |
| 50 | 146.7 | 390.1 | 633.5 |
| 55 | 270.6 | 439.0 | 607.3 |
| 60 | 298.6 | 461.6 | 624.7 |
| 65 | 313.9 | 468.8 | 623.6 |
| 70 | 342.1 | 473.2 | 604.2 |
| 75 | 311.8 | 465.9 | 620.0 |
| 80 | 308.6 | 457.6 | 606.5 |
| 85 | 325.9 | 464.6 | 603.3 |
| 90 | 283.6 | 513.5 | 743.3 |
| 95 | 255.8 | 654.1 | 1052.3 |
| 100 | 556.4 | 900.1 | 1243.8 |
| 105 | 598.8 | 923.2 | 1247.7 |
| 110 | 579.1 | 935.3 | 1291.4 |
| 115 | 662.1 | 1024.6 | 1387.2 |
| 120 | 687.2 | 1015.4 | 1343.6 |
| 125 | 715.9 | 1012.9 | 1309.9 |
| 130 | 730.2 | 1020.4 | 1310.6 |
| 135 | 738.5 | 1010.4 | 1282.2 |
| 140 | 759.1 | 1025.2 | 1291.4 |
| 145 | 771.9 | 1030.1 | 1288.4 |
| 150 | 768.1 | 1028.1 | 1288.1 |

In one example, the select catheter 400 may have a stiffness at about 100 mm from the distal tip 411 within an inclusive range of about 556.4 μm and 1243.8 gf. In another example, the select catheter 400 may have a stiffness at about 80 mm from a distal tip 411 of the select catheter within an inclusive range of about 308.6 gf and 606.5 gf. In another example, the select catheter 400 may have a stiffness at about 20 mm within an inclusive range of about 7.0 gf and 520.5 gf. In another example, the select catheter 400 may have a stiffness at about 10 mm within an inclusive range of about 12.4 gf and 76.1 gf. In another example, the select catheter 400 may have a stiffness at about 5 mm within an inclusive range of about 11.7 gf and 62.9 gf.

In another example, the select catheter 400 may have an average stiffness at about 100 mm from the distal tip 411 of about 900.1 gf, about 80 mm from the distal tip 411 of about 457.6, at about 20 mm from the distal tip 411 of about 192.9 gf, at about 10 mm from the distal tip 411 of about 44.2, and at about 5 mm from the distal tip 411 of about 37.3 gf.

As previously discussed, the three-dimensional shape, the low friction coating, and the stiffness profile may all be included in the same example catheter, or they may be used in any combination together, including being used alone.

There are many advantages and benefits to the disclosed embodiments and alternatives described herein. For example, they may reduce overall procedure time by reducing the time an operator spends attempting to gain access to the target vasculature. In many endovascular procedures, “time is brain,” meaning a faster procedure can result in quicker treatment (e.g., of stroke or aneurysm) and lead to improved neurological outcomes. It may also reduce the amount of surface contact and contact force between the catheter (here the select catheter) and the vasculature at or near the aortic arch. This may reduce the likelihood that the catheter scrapes or contacts a non-target vessel and dislodges a potential embolus (e.g., clot, sclerotic tissue, plaque, etc.). Thus, the likelihood of causing an unintended stroke or embolic event may be reduced. It may also allow for access to vasculature that would otherwise not be accessible due to tortuosity of the vessels or unique anatomies. These are exemplary benefits and advantages of these embodiments, and they should not be viewed as limiting examples. Other benefits and advantages to the patient, operator, and/or manufacturer are also contemplated.

In the example catheters and methods of this specification, it may be desirable to access a right vertebral artery either instead of or along with right common carotid artery access.

In the example catheters and methods of this specification, access via the left subclavian artery can be accessed with a wider memorized shape of the examples previously described for access via the right subclavian artery and narrower memorized shape widths for access to the left common carotid artery access.

### Claim Bank

Clause 1. A select catheter, comprising: an elongated catheter body comprising a distal region with a constrained linear shape and an unconstrained shape; wherein the distal region comprises a distal first section and a proximal second section that forms a major curve with the proximal second section in the unconstrained shape, wherein the distal first section is distally located relative to the proximal second section in the constrained linear shape; and, where in the unconstrained shape: the proximal second section lies substantially in a first reference plane; and, the distal first section is positioned at least partially outside of the first reference plane.

Clause 2. The select catheter of clause 1, wherein a distal tip of the distal first section is positioned proximally of the major curve.

Clause 3. The select catheter of clause 1, wherein the distal first section is positioned at an angle within an inclusive range of 5 and 25 degrees relative to the first reference plane.

Clause 4. The select catheter of clause 3, wherein the distal first section is positioned at an angle of about 11, 11.01, 11.02, 11.03, 11.04, 11.05, 11.06, 11.07, 11.08, 11.09, 11.1, 11.11, 11.12, 11.13, 11.14, 11.15, 11.16, 11.17, 11.18, 11.19, 11.2, 11.21, 11.22, 11.23, 11.24, 11.25, 11.26, 11.27, 11.28, 11.29, 11.3, 11.31, 11.32, 11.33, 11.34, 11.35, 11.36, 11.37, 11.38, 11.39, 11.4, 11.41, 11.42, 11.43, 11.44, 11.45, 11.46, 11.47, 11.48, 11.49, 11.5, 11.51, 11.52, 11.53, 11.54, 11.55, 11.56, 11.57, 11.58, 11.59, 11.6, 11.61, 11.62, 11.63, 11.64, 11.65, 11.66, 11.67, 11.68, 11.69, 11.7, 11.71, 11.72, 11.73, 11.74, 11.75, 11.76, 11.77, 11.78, 11.79, 11.8, 11.81, 11.82, 11.83, 11.84, 11.85, 11.86, 11.87, 11.88, 11.89, 11.9, 11.91, 11.92, 11.93, 11.94, 11.95, 11.96, 11.97, 11.98, 11.99, or 12 degrees relative to the first reference plane.

Clause 5. The select catheter of clause 3, wherein a distal tip of the distal first section is positioned at about inclusive range of about 0.4 cm to 2.0 cm away from a face of the first reference plane.

Clause 6. The select catheter of clause 5, wherein the distal tip of the distal first section is positioned at a length from the proximal second section that is about parallel to the first reference plane within an inclusive range of about 1 cm to about 2 cm away from the proximal second section.

Clause 7. The select catheter of clause 1, wherein the distal first section further comprises a first minor curve.

Clause 8. The select catheter of clause 7, wherein the first minor curve curves in a direction generally away from the proximal second section.

Clause 9. The select catheter of clause 7, wherein the first minor curve has a curvature less than a curvature of the major curve.

Clause 10. The select catheter of clause 7, wherein the first minor curve has a curvature within an inclusive range of about 90 to about 190 degrees.

Clause 11. The select catheter of clause 1, wherein the distal first section is within an inclusive range of about 3 cm to 8 cm in length.

Clause 12. The select catheter of clause 9, wherein the distal first section is positioned at an angle within an inclusive range of 5 and 25 degrees relative to the first reference plane; wherein a distal tip of the distal first section is positioned at about inclusive range of about 0.4 cm to 2.0 cm away from a face of the first reference plane; wherein the distal tip of the distal first section is positioned at a length from the proximal second section that is about parallel to the first reference plane within an inclusive range of about 1 cm to about 2 cm away from the proximal second section; wherein the first minor curve has a curvature within an inclusive range of about 90 to about 190 degrees; wherein the distal first section is within an inclusive range of about 3 cm to 8 cm in length.

Clause 13. The select catheter of clause 1, wherein the select catheter has a stiffness at about 110 mm from a distal tip of the elongated catheter body of about 136.76 gf, about 80 mm from the distal tip of about 110.34 gf, at about 20 mm from the distal tip of about 22.58 gf, at about 10 mm from the distal tip of about 13.5 gf, and at about 5 mm from the distal tip of about 8 gf.

Clause 14. The select catheter of clause 1, wherein the select catheter has a stiffness at about 10 mm within a range of about 6.75 gf and 20.25 gf.

Clause 15. The select catheter of clause 14, wherein the select catheter has a stiffness at about 20 mm within a range of about 11.29 gf and 33.86 gf.

Clause 16. The select catheter of clause 3, wherein the distal first section is positioned at an angle of about 10.0, 10.05, 10.1, 10.15, 10.2, 10.25, 10.3, 10.35, 10.4, 10.45, 10.5, 10.55, 10.6, 10.65, 10.7, 10.75, 10.8, 10.85, 10.9, 10.95, or 11.0 degrees relative to the first reference plane.

Clause 17. The select catheter of clause 3, wherein a distal tip of the distal first section is positioned within inclusive range of about 0.1 cm to about 1.0 cm away from a face of the first reference plane.

Clause 18. The select catheter of clause 17, wherein the distal tip of the distal first section is positioned at a length from the proximal second section that is about parallel to the first reference plane within an inclusive range of about .2 cm to about .5 cm away from the proximal second section.

Clause 19. The select catheter of clause 1, wherein the distal first section is within an inclusive range of about 2 cm to 5 cm in length.

Clause 20. The select catheter of clause 9, wherein the distal first section is positioned at an angle within an inclusive range of 5 and 25 degrees relative to the first reference plane; wherein a distal tip of the distal first section is positioned at about inclusive range of about 0.1 cm to 1.0 cm away from a face of the first reference plane; wherein the distal tip of the distal first section is positioned at a length from the proximal second section that is about parallel to the first reference plane within an inclusive range of about .2 cm to about .5 cm away from the proximal second section; wherein the first minor curve has a curvature within an inclusive range of about 90 to about 190 degrees; wherein the distal first section is within an inclusive range of about 2 cm to 5 cm in length.

Clause 21. The select catheter of clause 1, wherein the select catheter has a stiffness at about 110 mm from a distal tip of the elongated catheter body of about 154.85 gf, about 80 mm from the distal tip of about 128.99 gf, at about 20 mm from the distal tip of about 38.96 gf, at about 10 mm from the distal tip of about 12.08 gf, and at about 5 mm from the distal tip of about 6.40 gf.

Clause 22. The select catheter of clause 1, wherein the select catheter has a stiffness at about 10 mm within a range of about 6.04 gf and 18.11 gf.

Clause 23. The select catheter of clause 14, wherein the select catheter has a stiffness at about 20 mm within a range of about 19.48 gf and 58.44 gf.

Clause 24. A select catheter, comprising: an elongated catheter body comprising a distal region with an unconstrained shape; wherein the distal region comprises a first distal section that forms a major curve with a second distal section in the unconstrained shape; and, wherein the unconstrained shape the first distal section is positioned at a non-parallel angle relative to the second distal section and wherein a free distal tip of the distal region is positioned in a generally proximal orientation.

Clause 25. A select catheter, comprising: an elongated catheter body comprising a distal region with an unconstrained shape means for accessing a right common carotid artery or left common carotid artery via radial access; wherein the distal region comprises a first distal section that forms a major curve with a second distal section.

Clause 26. A select catheter, comprising: an elongated catheter body; wherein the elongated catheter body has a stiffness at about 110 mm from a distal tip within an inclusive range of about 68.38 gf and 205.14 gf; a stiffness at about 80 mm from a distal tip within an inclusive range of about 55.17 gf and 165.51 gf; a stiffness at about 20 mm within an inclusive range of about 11.29 gf and 33.86 gf; a stiffness at about 10 mm within an inclusive range of about 6.75 gf and 20.25 gf; and a stiffness at about 5 mm within an inclusive range of about 4 gf and 12 gf.

Clause 27. The select catheter of clause 26, wherein the elongated catheter body has a stiffness at about 110 mm from the distal tip of about 136.76 gf, about 80 mm from the distal tip of about 110.34 gf, at about 20 mm from the distal tip of about 22.58 gf, at about 10 mm from the distal tip of about 13.5 gf, and at about 5 mm from the distal tip of about 8 gf.

Clause 28. A select catheter, comprising: an elongated catheter body; wherein the elongated catheter body has a stiffness at about 110 mm from a distal tip within an inclusive range of about 77.42 gf and 232.27 gf; a stiffness at about 80 mm from the distal tip of the select catheter within an inclusive range of about 64.50 gf and 193.49 gf; a stiffness at about 20 mm from the distal tip within an inclusive range of about 19.48 gf and 58.44 gf; a stiffness at about 10 mm from the distal tip within an inclusive range of about 6.04 gf and 18.11 gf; and a stiffness at about 5 mm from the distal tip within an inclusive range of about 3.20 gf and 9.60 gf.

Clause 29. The select catheter of clause 28, wherein the elongated catheter body has a stiffness at about 110 mm from the distal tip of about 154.85 gf, about 80 mm from the distal tip of about 128.99 gf, about 20 mm from the distal tip of about 38.96 gf, about 10 mm from the distal tip of about 12.08 gf, and about 5 mm from the distal tip of about 6.40 gf.

Clause 30. A select catheter, comprising: an elongated catheter body; wherein the elongated catheter body has a stiffness of either of the following: wherein the elongated catheter body has a stiffness at about 110 mm from a distal tip of the elongated catheter body of about 136.76 gf, about 80 mm from the distal tip of about 110.34 gf, at about 20 mm from the distal tip of about 22.58 gf, at about 10 mm from the distal tip of about 13.5 gf, and at about 5 mm from the distal tip of about 8 gf; or, wherein the elongated catheter body has a stiffness at about 110 mm from the distal tip of about 154.85 gf, about 80 mm from the distal tip of about 128.99 gf, about 20 mm from the distal tip of about 38.96 gf, about 10 mm from the distal tip of about 12.08 gf, and about 5 mm from the distal tip of about 6.40 gf.

Clause 31. A catheter, comprising: an elongated catheter body having a proximal portion and a distal portion; wherein the distal portion forms a memorized shape when unconstrained; the memorized shape comprising a plurality of curves positioned to orient the distal portion within a right or left subclavian artery or brachiocephalic artery to access a left or right common carotid artery.

Clause 32. The catheter of clause 31, wherein the memorized shape of the distal portion is a “U” shape further curved in a generally perpendicular direction relative to an apex of the “U” shape.

Clause 33. The catheter of clause 31, wherein the memorized shape has dimensions within a following inclusive ranges: a length of about 3.0 cm to 8.5 cm, a width of about 1.5 cm to 3.0 cm, and a height of about 1.5 cm to 3.5 cm.

Clause 34. The catheter of clause 31, wherein the distal portion further comprises a proximal region, an intermediate region, and a distal region, and wherein a first curve of the plurality of curves is located between the proximal region and the intermediate region and has an angle of curvature within an inclusive range of about 35 degrees to about 75 degrees.

Clause 35. The catheter of clause 34, wherein a second curve of the plurality of curves is located between the intermediate region and the distal region and has an angle of curvature within an inclusive range of about 160 degrees to about 200 degrees.

Clause 36. The catheter of clause 35, wherein some of the plurality of curves are located within the distal region and have angles of curvature within an inclusive range of about 5 degrees to 30 degrees.

Clause 37. A method for accessing a left common carotid artery, comprising: advancing a distal portion of a select catheter from a right subclavian artery and into an aortic arch of a patient; allowing the distal portion of the select catheter to expand to a memorized shape in the aortic arch of the patient; and proximally retracting some of distal portion of the select catheter partially back into the right subclavian artery or a brachiocephalic artery such that a distal region of the distal portion moves into the left common carotid artery.

Clause 38. The method of clause 37, wherein the memorized shape of the distal portion is a “U” shape further curved in a generally perpendicular direction relative to an apex of the “U” shape.

Clause 39. A catheter, comprising: an elongated catheter body having a proximal portion and a distal portion; wherein the distal portion forms a memorized shape when unconstrained; the memorized shape comprising a plurality of curves positioned to orient the distal portion within a right subclavian artery or brachiocephalic artery and also into a right common carotid artery.

Clause 40. The catheter of clause 39, wherein the memorized shape of the distal portion is a “U” shape having a width within an inclusive range of about 0.7 cm to about 2.9 cm.

Clause 41. The catheter of clause 40, wherein the distal portion includes a first curve with an angle of curvature within an inclusive range of about 70 degrees to 140 degrees, as well as a second curve, third curve, and a fourth curve that each have angles of curvature within an inclusive range of about 50-140 degrees.

Clause 42. A method for accessing a right common carotid artery, comprising: advancing a distal portion of a select catheter into a right subclavian artery or a brachiocephalic artery of a patient; further distally advancing the distal portion of the select catheter so that it curves to a memorized shape in the right subclavian artery or the brachiocephalic artery; and further distally advancing the curved memorized shape so that a portion of the distal portion enters the right common carotid artery.

Clause 43. The method of clause 42, wherein the memorized shape of the distal portion is a “U” shape having a width within an inclusive range of about 0.7 cm to about 2.9 cm.

Clause 44. A catheter, comprising: an elongated catheter body having a proximal portion and a distal portion; wherein the distal portion forms a memorized shape when unconstrained; the memorized shape comprising a primary curve having an angle of curvature within an inclusive range of about 335 to about 350 degrees positioned to orient the distal portion within a right subclavian artery or brachiocephalic artery and also into a right common carotid artery.

Clause 45. The catheter of clause 44, wherein the primary curve has a width within an inclusive range of about 0.5 cm to about 2.5 cm.

Clause 46. A catheter, comprising: an elongated catheter body having a proximal portion and a distal portion; wherein the distal portion has a constrained configuration and an unconstrained configuration different from the constrained configuration, and wherein in the unconstrained configuration, the catheter shape comprises a plurality of curves that deflect at least in part relative to an axial “X” axis through a central lumen of the proximal portion of the catheter, a vertical “Y” axis perpendicular to the “X” axis, and a lateral “Z” axis perpendicular to both the “X” and “Y” axes.

Clause 47. The catheter of clause 46, wherein the plurality of curves are positioned to orient the distal portion of the catheter for access to a specific target vessel.

Clause 48. The catheter of clause 47, wherein the specific target vessel is one of a right or left common carotid artery.

Clause 49. The catheter of clause 46, wherein the distal portion of the catheter in the unconstrained configuration has a memorized shape, and wherein at least a portion of the memorized shape of the distal portion comprises a “U” shape in the unconstrained configuration, wherein the “U” shape has a proximal section, a medial section, and a distal section, wherein the proximal section of the “U” shape extends at least in part along the “X” axis away from the proximal portion of the catheter, and wherein the distal section of the “U” shape extends at least in part along the “X” axis towards the proximal portion of the catheter.

Clause 50. A catheter, comprising: an elongated catheter means for accessing a common carotid artery, wherein the catheter means has a body with a proximal portion and a distal portion; wherein the distal portion has a constrained configuration and an unconstrained configuration different from the constrained configuration, and wherein the elongated catheter means forms a memorized shape when unconstrained, the memorized shape comprising a plurality of curves to orient the distal portion within a right or left subclavian artery or brachiocephalic artery to access a left common carotid artery.

1. A select catheter, comprising:
an elongated catheter body comprising a distal region with a constrained linear shape and an unconstrained shape;
wherein the distal region comprises a distal first section and a proximal second section that forms a major curve with the proximal second distal first section in the unconstrained shape, wherein the distal first section is distally located relative to the proximal second section in the constrained linear shape; and,
where in the unconstrained shape:
the proximal second section lies substantially in a first reference plane;
the distal first section is positioned at least partially outside of the first reference plane; and,
the distal first section and the proximal second section do not lie substantially entirely in a common plane.

2. The select catheter of claim 1, wherein a distal tip of the distal first section is positioned proximally of the major curve.

3. The select catheter of claim 1, wherein the distal first section is positioned at an angle within an inclusive range of 5 and 25 degrees relative to the first reference plane.

4. The select catheter of claim 3, wherein the distal first section is positioned at an angle of about 11, 11.01, 11.02, 11.03, 11.04, 11.05, 11.06, 11.07, 11.08, 11.09, 11.1, 11.11, 11.12, 11.13, 11.14, 11.15, 11.16, 11.17, 11.18, 11.19, 11.2, 11.21, 11.22, 11.23, 11.24, 11.25, 11.26, 11.27, 11.28, 11.29, 11.3, 11.31, 11.32, 11.33, 11.34, 11.35, 11.36, 11.37, 11.38, 11.39, 11.4, 11.41, 11.42, 11.43, 11.44, 11.45, 11.46, 11.47, 11.48, 11.49, 11.5, 11.51, 11.52, 11.53, 11.54, 11.55, 11.56, 11.57, 11.58, 11.59, 11.6, 11.61, 11.62, 11.63, 11.64, 11.65, 11.66, 11.67, 11.68, 11.69, 11.7, 11.71, 11.72, 11.73, 11.74, 11.75, 11.76, 11.77, 11.78, 11.79, 11.8, 11.81, 11.82, 11.83, 11.84, 11.85, 11.86, 11.87, 11.88, 11.89, 11.9, 11.91, 11.92, 11.93, 11.94, 11.95, 11.96, 11.97, 11.98, 11.99, or 12 degrees relative to the first reference plane.

5. The select catheter of claim 3, wherein a distal tip of the distal first section is positioned at about inclusive range of about 0.4 cm to 2.0 cm away from a face of the first reference plane.

6. The select catheter of claim 5, wherein the distal tip of the distal first section is positioned at a length from the proximal second section that is about parallel to the first reference plane within an inclusive range of about 1 cm to about 2 cm away from the proximal second section.

7. The select catheter of claim 1, wherein the distal first section further comprises a first minor curve.

8. The select catheter of claim 7, wherein the first minor curve curves in a direction generally away from the proximal second section.

9. The select catheter of claim 7, wherein the first minor curve has a curvature less than a curvature of the major curve.

10. The select catheter of claim 7, wherein the first minor curve has a curvature within an inclusive range of about 90 to about 190 degrees.

11. The select catheter of claim 1, wherein the distal first section is within an inclusive range of about 3 cm to 8 cm in length.

12. The select catheter of claim 9, wherein the distal first section is positioned at an angle within an inclusive range of 5 and 25 degrees relative to the first reference plane; wherein a distal tip of the distal first section is positioned at about inclusive range of about 0.4 cm to 2.0 cm away from a face of the first reference plane; wherein the distal tip of the distal first section is positioned at a length from the proximal second section that is about parallel to the first reference plane within an inclusive range of about 1 cm to about 2 cm away from the proximal second section; wherein the first minor curve has a curvature within an inclusive range of about 90 to about 190 degrees; wherein the distal first section is within an inclusive range of about 3 cm to 8 cm in length.

13. The select catheter of claim 1, wherein the select catheter has a stiffness at about 110 mm from a distal tip of the elongated catheter body of about 136.76 gf, about 80 mm from the distal tip of about 110.34 gf, at about 20 mm from the distal tip of about 22.58 gf, at about 10 mm from the distal tip of about 13.5 gf, and at about 5 mm from the distal tip of about 8 gf.

14. The select catheter of claim 1, wherein the select catheter has a stiffness at about 10 mm within a range of about 6.75 gf and 20.25 gf.

15. The select catheter of claim 14, wherein the select catheter has a stiffness at about 20 mm within a range of about 11.29 gf and 33.86 gf.

16. The select catheter of claim 3, wherein the distal first section is positioned at an angle of about 10.0, 10.05, 10.1, 10.15, 10.2, 10.25, 10.3, 10.35, 10.4, 10.45, 10.5, 10.55, 10.6, 10.65, 10.7, 10.75, 10.8, 10.85, 10.9, 10.95, or 11.0 degrees relative to the first reference plane.

17. The select catheter of claim 3, wherein a distal tip of the distal first section is positioned within inclusive range of about 0.1 cm to about 1.0 cm away from a face of the first reference plane.

18. The select catheter of claim 17, wherein the distal tip of the distal first section is positioned at a length from the proximal second section that is about parallel to the first reference plane within an inclusive range of about 2 cm to about .5 cm away from the proximal second section.

19. The select catheter of claim 1, wherein the distal first section is within an inclusive range of about 2 cm to 5 cm in length.

20. The select catheter of claim 9, wherein the distal first section is positioned at an angle within an inclusive range of 5 and 25 degrees relative to the first reference plane; wherein a distal tip of the distal first section is positioned at about inclusive range of about 0.1 cm to 1.0 cm away from a face of the first reference plane; wherein the distal tip of the distal first section is positioned at a length from the proximal second section that is about parallel to the first reference plane within an inclusive range of about .2 cm to about .5 cm away from the proximal second section; wherein the first minor curve has a curvature within an inclusive range of about 90 to about 190 degrees; wherein the distal first section is within an inclusive range of about 2 cm to 5 cm in length.

21. The select catheter of claim 1, wherein the select catheter has a stiffness at about 110 mm from a distal tip of the elongated catheter body of about 154.85 gf, about 80 mm from the distal tip of about 128.99 gf, at about 20 mm from the distal tip of about 38.96 gf, at about 10 mm from the distal tip of about 12.08 gf, and at about 5 mm from the distal tip of about 6.40 gf.

22. The select catheter of claim 1, wherein the select catheter has a stiffness at about 10 mm within a range of about 6.04 gf and 18.11 gf.

23. The select catheter of claim 14, wherein the select catheter has a stiffness at about 20 mm within a range of about 19.48 gf and 58.44 gf.

24. A select catheter, comprising:
an elongated catheter body comprising a distal region with an unconstrained shape;
wherein the distal region comprises a first distal section that forms a major curve with a second distal section in the unconstrained shape; and,
wherein the unconstrained shape of the first distal section is positioned at a non-parallel angle relative to the second distal section and wherein a free distal tip of the distal region is positioned in a generally proximal orientation;
wherein the first distal section and the second distal section do not lie substantially entirely in a common plane.

25. A select catheter, comprising:
an elongated catheter body comprising a distal region with an unconstrained shape means for accessing a right common carotid artery or left common carotid artery via radial access;
wherein the distal region comprises a first distal section that forms a major curve with a second distal section;
wherein the first distal section and the second distal section do not lie substantially entirely in a common plane.

26-30. (canceled)
